(19) United States
(12) Patent Application Publication
(10) Pub. No.: US 2024/0023431 A1
(43) Pub. Date: Jan. 18, 2024

(54) Organic Compound, Light-Emitting Device, Light-Emitting Apparatus, Electronic Apparatus and Lighting Device

(71) Applicant: Semiconductor Energy Laboratory Co., Ltd., Kanagawa-ken (JP)

(72) Inventors: Takeyoshi WATABE, Atsugi,Kanagawa (JP); Airi UEDA, Zama, Kanagawa (JP); Kyoko TAKEDA, Atsugi, Kanagawa (JP); Nobuharu OHSAWA, Zama, Kanagawa (JP); Satoshi SEO, Sagamihara. Kanagawa (JP); Tomohiro KUBOTA, Atsugi, Kanagawa (JP); Takashi HIRAHARA, Atsugi, Kanagawa (JP)

(73) Assignee: Semiconductor Energy Laboratory Co., Ltd., Kanagawa-ken (JP)

(21) Appl. No.: 18/013,374

(22) PCT Filed: Jun. 23, 2021
(86) PCT No.: PCT/IB2021/055520

§ 371 Date: Dec. 28, 2022

(30) Foreign Application Priority Data
Jul. 3, 2020 (JP) 2020-115943

(51) Int. Cl. H10K 85/60; C07D 409/12; C09K 11/06; C07D 405/12; C07C 211/61

(52) U.S. Cl. CPC H10K 85/636; H10K 50/17; C09K 11/06; C07D 405/12; C07C 211/61; H10K 85/633; C09K 2211/1018; H10K 85/6572; H10K 85/6576; H10K 85/6574; C07C 2603/18; C09K 2211/1007; C09K 2211/1011; C09K 2211/1014; H10K 85/615; C07D 409/12

(57) ABSTRACT

Provided is a light-emitting device with high emission efficiency. Provided is a light-emitting device including an anode, a cathode, and an EL layer positioned therebetween; the EL layer includes a light-emitting layer, a first layer, and a second layer; the first layer is positioned between the anode and the light-emitting layer; the first layer is in contact with the second layer; the second layer contains a monoamine compound having an arylamine structure; a first group, a second group, and a third group are bonded to a nitrogen atom included in the amine in the monoamine compound; the first group is a group including a carbazole structure; the second group is a group including a dibenzofuran structure or a dibenzothiophene structure; the third group includes an aromatic hydrocarbon structure having 6 to 18 carbon atoms or a heteroaromatic hydrocarbon structure having 4 to 26 carbon atoms; and a refractive index of the first layer is lower than a refractive index of the light-emitting layer.

### TECHNICAL FIELD

One embodiment of the present invention relates to an organic compound, a light-emitting element, a light-emitting device, a display module, a lighting module, a display device, a light-emitting apparatus, an electronic apparatus, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting apparatus, a lighting device, a power storage device, a memory device, an imaging device, a driving method thereof, and a manufacturing method thereof.

### BACKGROUND ART

Light-emitting devices (organic EL devices) including organic compounds and utilizing electroluminescence (EL) have been put to more practical use. In the basic structure of such light-emitting devices, an organic compound layer containing a light-emitting material (an EL layer) is sandwiched between a pair of electrodes. Carriers are injected by application of a voltage to the device, and recombination energy of the carriers is used, whereby light emission can be obtained from the light-emitting material.

Such light-emitting devices are of self-luminous type and thus have advantages over liquid crystal displays, such as high visibility and no need for a backlight when used as pixels of a display, and are particularly suitable for flat panel displays. Displays including such light-emitting devices are also highly advantageous in that they can be thin and lightweight. Moreover, such light-emitting devices also have a feature that the response speed is extremely fast.

Since light-emitting layers of such light-emitting devices can be successively formed two-dimensionally, planar light emission can be achieved. This feature is difficult to realize with point light sources typified by incandescent lamps or LEDs or linear light sources typified by fluorescent lamps; thus, such light-emitting devices also have a great potential as planar light sources which can be applied to lighting devices and the like.

Displays or lighting devices including light-emitting devices are suitable for a variety of electronic apparatuses as described above, and research and development of light-emitting devices have progressed for more favorable characteristics.

Low outcoupling efficiency is often a problem in an organic EL device. In order to improve the outcoupling efficiency, a structure including a layer formed using a low refractive index material in an EL layer (see Non-Patent Document 1, for example) has been proposed.

### REFERENCE

### Non-Patent Document

[Non-Patent Document 1] Jaeho Lee and 12 others, “Synergetic electrode architecture for efficient graphene-based flexible organic light-emitting diodes”, nature COMMUNICATIONS, Jun. 2, 2016, DOI: 10. 1038/ncomms 11791.

### SUMMARY OF THE INVENTION

### Problems to be Solved by the Invention

An object of one embodiment of the present invention is to provide a light-emitting device with high emission efficiency. Another object of another embodiment of the present invention is to provide a light-emitting device with a low driving voltage. Another object of another embodiment of the present invention is to provide a light-emitting device with a favorable lifetime. Another object of another embodiment of the present invention is to provide any of a light-emitting device, a light-emitting apparatus, an electronic apparatus, a display device, and an electronic device each having low power consumption. Another object of another embodiment of the present invention is to provide any of a light-emitting device, a light-emitting apparatus, an electronic apparatus, a display device, and an electronic device each having favorable reliability.

It is only necessary that any one of the above-described objects be achieved in the present invention.

### Means for Solving the Problems

One embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; the second layer contains a first organic compound having an arylamine structure; a first group, a second group, and a third group are bonded to a nitrogen atom included in the amine in the first organic compound; the first group is a group including a carbazole structure; the second group is a group including a dibenzofuran structure or a dibenzothiophene structure; the third group includes an aromatic hydrocarbon structure having 6 to 18 carbon atoms or a heteroaromatic hydrocarbon structure having 4 to 26 carbon atoms; and a refractive index of the first layer is lower than a refractive index of the light-emitting layer.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; the second layer contains a first organic compound having an arylamine structure; a first group, a second group, and a third group are bonded to a nitrogen atom included in the amine in the first organic compound; the first group is a group including a carbazole structure; the second group is a group including a dibenzofuran structure or a dibenzothiophene structure; the third group includes an aromatic hydrocarbon structure having 6 to 18 carbon atoms or a heteroaromatic hydrocarbon structure having 4 to 26 carbon atoms; and an ordinary refractive index of the first layer with respect to light with a wavelength greater than or equal to 455 nm and less than or equal to 465 nm is higher than or equal to 1.5 and lower than or equal to 1.75.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; the second layer contains a first organic compound having an arylamine structure; a first group, a second group, and a third group are bonded to a nitrogen atom included in the amine in the first organic compound; the first group is a group including a carbazole structure; the second group is a group including a dibenzofuran structure or a dibenzothiophene structure; the third group includes an aromatic hydrocarbon structure having 6 to 18 carbon atoms or a heteroaromatic hydrocarbon structure having 4 to 26 carbon atoms; and a refractive index of the first layer with respect to light with a wavelength of 633 nm is higher than or equal to 1.45 and lower than or equal to 1.70.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; the second layer contains a first organic compound having a triarylamine structure; a first group, a second group, and a third group are bonded to a nitrogen atom included in the amine in the first organic compound; the first group is a group including a carbazole structure; the second group is a group including a dibenzofuran structure or a dibenzothiophene structure; the third group includes an aromatic hydrocarbon structure having 6 to 18 carbon atoms or a heteroaromatic hydrocarbon structure having 4 to 26 carbon atoms; the first layer contains an organic compound having a hole-transport property; and an ordinary refractive index of the organic compound having a hole-transport property with respect to light with a wavelength greater than or equal to 455 nm and less than or equal to 465 nm is higher than or equal to 1.5 and lower than or equal to 1.75.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; the second layer contains a first organic compound having a triarylamine structure; a first group, a second group, and a third group are bonded to a nitrogen atom included in the amine in the first organic compound; the first group is a group including a carbazole structure; the second group is a group including a dibenzofuran structure or a dibenzothiophene structure; the third group includes an aromatic hydrocarbon structure having 6 to 18 carbon atoms or a heteroaromatic hydrocarbon structure having 4 to 26 carbon atoms; the first layer contains an organic compound having a hole-transport property; and a refractive index of the organic compound having a hole-transport property with respect to light with a wavelength of 633 nm is higher than or equal to 1.45 and lower than or equal to 1.70.

Another embodiment of the present invention is the light-emitting device having the above structure, in which the organic compound having a hole-transport property includes a plurality of alkyl groups.

Another embodiment of the present invention is the light-emitting device having the above structure, in which the carbazole structure of the first group has a bonding site at any of a 2-position, a 3-position, and a 9-position; and the carbazole structure is bonded to the nitrogen atom through the bonding site or the bonding site and a divalent aromatic hydrocarbon group.

Another embodiment of the present invention is the light-emitting device having the above structure, in which the carbazole structure of the first group has a bonding site at a 2-position or a 3-position; and the carbazole structure is bonded to the nitrogen atom through the bonding site or the bonding site and a divalent aromatic hydrocarbon group.

Another embodiment of the present invention is the light-emitting device having the above structure, in which the carbazole structure of the first group has a bonding site at a 2-position; and the carbazole structure is bonded to the nitrogen atom through the bonding site or the bonding site and a divalent aromatic hydrocarbon group.

Another embodiment of the present invention is the light-emitting device having the above structure, in which the divalent aromatic hydrocarbon group is a phenylene group.

Another embodiment of the present invention is the light-emitting device having the above structure, in which each of the dibenzofuran structure and the dibenzothiophene structure of the second group is bonded to the nitrogen atom through a divalent aromatic hydrocarbon group.

Another embodiment of the present invention is the light-emitting device having the above structure, in which the divalent aromatic hydrocarbon group included in the second group is a phenylene group or a biphenyldiyl group.

Another embodiment of the present invention is the light-emitting device having the above structure, in which a positional relationship between bonding sites of the phenylene group or bonding sites of at least one benzene structure of the biphenyldiyl group is a meta-position.

Another embodiment of the present invention is the light-emitting device having the above structure, in which the third group is a biphenyl group or a terphenyl group.

Another embodiment of the present invention is the light-emitting device having the above structure, in which the third group is a group including a dibenzofuran structure or a dibenzothiophene structure.

Another embodiment of the present invention is the light-emitting device having the above structure, in which the second layer is positioned between the first layer and the light-emitting layer.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; a refractive index of the first layer is lower than a refractive index of the light-emitting layer; and the second layer contains an organic compound represented by General Formula (G1) below.

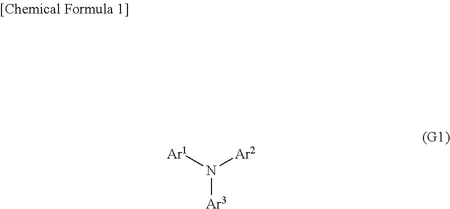

Note that in General Formula (G1) above, Ar¹ is a group represented by General Formula (g1) below, Ar² is a group represented by General Formula (g2) or (g3) below, and Ar³ is any of a group represented by General Formula (g1) below and an aromatic hydrocarbon group having 6 to 18 carbon atoms.

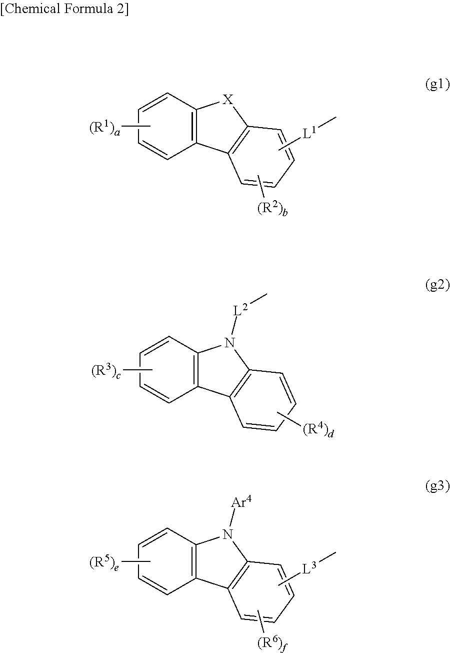

Note that in General Formulae (g1) to (g3) above, each of R¹ to R⁶ is independently any of a hydrocarbon group having 1 to 6 carbon atoms and an aromatic hydrocarbon group having 6 to 13 carbon atoms, and Ar⁴ is a substituted or unsubstituted phenyl group. Furthermore, each of a, c, d, and e independently represents an integer of 0 to 4, and each of b and f independently represents an integer of 0 to 3. Furthermore, each of L¹ to L³ independently represents a divalent aromatic hydrocarbon group having 6 to 12 carbon atoms, and X is an oxygen atom or a sulfur atom.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; an ordinary refractive index of the first layer with respect to light with a wavelength greater than or equal to 455 nm and less than or equal to 465 nm is higher than or equal to 1.5 and lower than or equal to 1.75; and the second layer contains an organic compound represented by General Formula (G1) below.

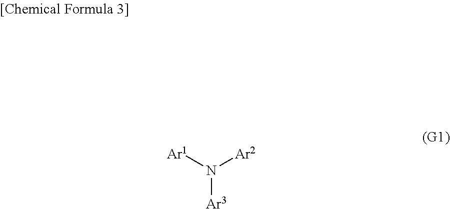

Note that in General Formula (G1) above, Ar¹ is a group represented by General Formula (g1) below, Ar² is a group represented by General Formula (g2) or (g3) below, and Ar³ is any of a group represented by General Formula (g1) below and an aromatic hydrocarbon group having 6 to 18 carbon atoms.

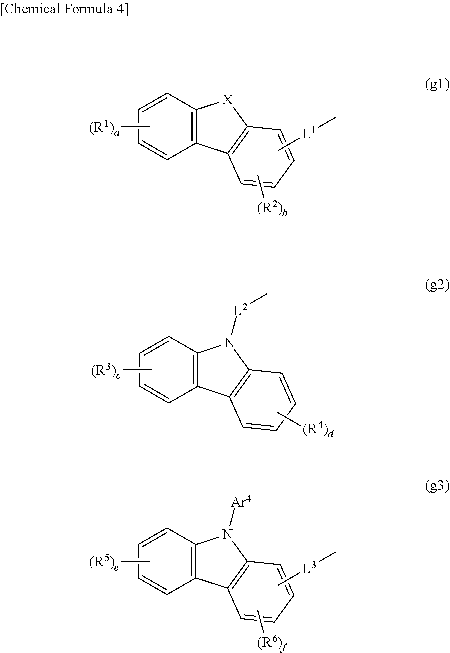

Note that in General Formulae (g1) to (g3) above, each of R¹ to R⁶ is independently any of a hydrocarbon group having 1 to 6 carbon atoms and an aromatic hydrocarbon group having 6 to 13 carbon atoms, and Ar⁴ is a substituted or unsubstituted phenyl group. Furthermore, each of a, c, d, and e independently represents an integer of 0 to 4, and each of b and f independently represents an integer of 0 to 3. Furthermore, each of L¹ to L³ independently represents a divalent aromatic hydrocarbon group having 6 to 12 carbon atoms, and X is an oxygen atom or a sulfur atom.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; a refractive index of the first layer with respect to light with a wavelength of 633 nm is higher than or equal to 1.45 and lower than or equal to 1.70; and the second layer contains an organic compound represented by General Formula (G1) below.

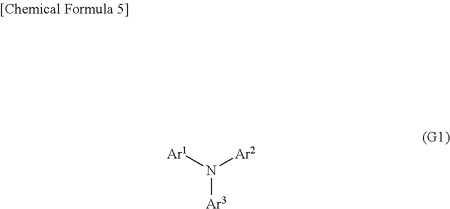

Note that in General Formula (G1) above, Ar¹ is a group represented by General Formula (g1) below, Ar² is a group represented by General Formula (g2) or (g3) below, and Ar³ is any of a group represented by General Formula (g1) below and an aromatic hydrocarbon group having 6 to 18 carbon atoms.

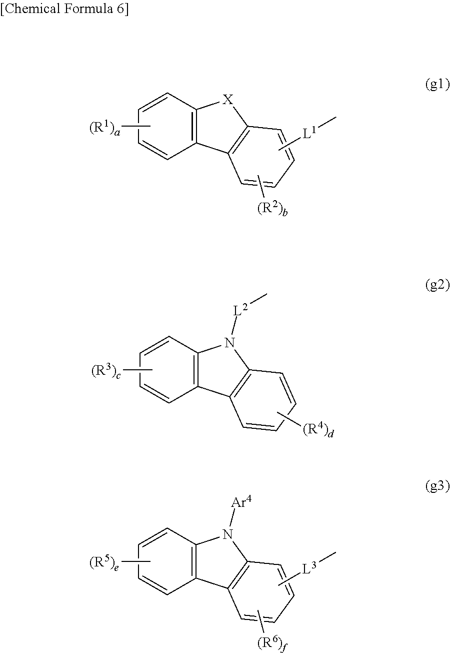

Note that in General Formulae (g1) to (g3) above, each of R¹ to R⁶ is independently any of a hydrocarbon group having 1 to 6 carbon atoms and an aromatic hydrocarbon group having 6 to 13 carbon atoms, and Ar⁴ is a substituted or unsubstituted phenyl group. Furthermore, each of a, c, d, and e independently represents an integer of 0 to 4, and each of b and f independently represents an integer of 0 to 3. Furthermore, each of L¹ to L³ independently represents a divalent aromatic hydrocarbon group having 6 to 12 carbon atoms, and X is an oxygen atom or a sulfur atom.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; the first layer contains an organic compound having a hole-transport property; an ordinary refractive index of the organic compound having a hole-transport property with respect to light with a wavelength greater than or equal to 455 nm and less than or equal to 465 nm is higher than or equal to 1.5 and lower than or equal to 1.75; and the second layer contains an organic compound represented by General Formula (G1) below.

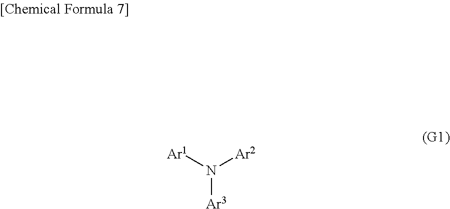

Note that in General Formula (G1) above, Ar¹ is a group represented by General Formula (g1) below, Ar² is a group represented by General Formula (g2) or (g3) below, and Ar³ is any of a group represented by General Formula (g1) below and an aromatic hydrocarbon group having 6 to 18 carbon atoms.

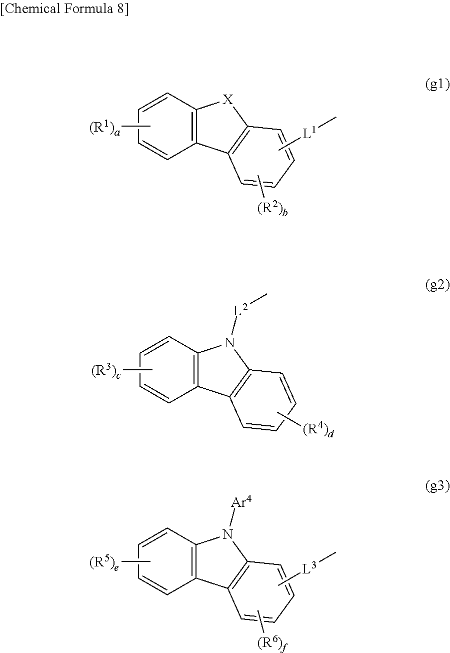

Note that in General Formulae (g1) to (g3) above, each of R¹ to R⁶ is independently any of a hydrocarbon group having 1 to 6 carbon atoms and an aromatic hydrocarbon group having 6 to 13 carbon atoms, and Ar⁴ is a substituted or unsubstituted phenyl group. Furthermore, each of a, c, d, and e independently represents an integer of 0 to 4, and each of b and f independently represents an integer of 0 to 3. Furthermore, each of L¹ to L³ independently represents a divalent aromatic hydrocarbon group having 6 to 12 carbon atoms, and X is an oxygen atom or a sulfur atom.

Another embodiment of the present invention is a light-emitting device including an anode, a cathode, and an EL layer positioned between the anode and the cathode; the EL layer includes a light-emitting layer, a first layer, and a second layer; the first layer is positioned between the anode and the light-emitting layer; the first layer is in contact with the second layer; the first layer contains an organic compound having a hole-transport property; a refractive index of the organic compound having a hole-transport property with respect to light with a wavelength of 633 nm is higher than or equal to 1.45 and lower than or equal to 1.70; and the second layer contains an organic compound represented by General Formula (G1) below.

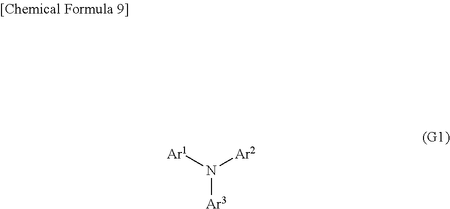

Note that in General Formula (G1) above, Ar¹ is a group represented by General Formula (g1) below, Ar² is a group represented by General Formula (g2) or (g3) below, and Ar³ is any of a group represented by General Formula (g1) below and an aromatic hydrocarbon group having 6 to 18 carbon atoms.

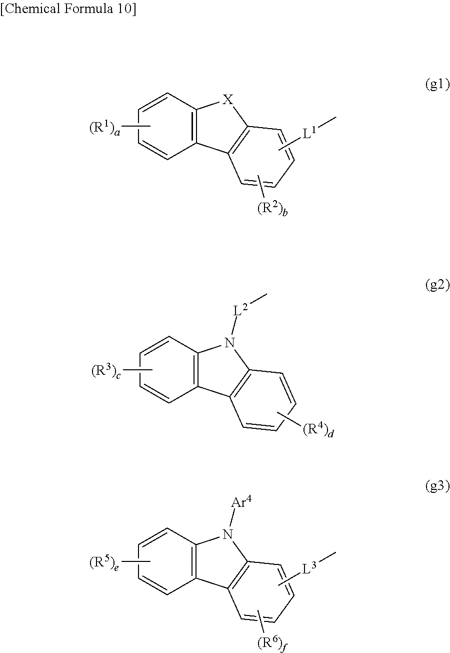

Note that in General Formulae (g1) to (g3) above, each of R¹ to R⁶ is independently any of a hydrocarbon group having 1 to 6 carbon atoms and an aromatic hydrocarbon group having 6 to 13 carbon atoms, and Ar⁴ is a substituted or unsubstituted phenyl group. Furthermore, each of a, c, d, and e independently represents an integer of 0 to 4, and each of b and f independently represents an integer of 0 to 3. Furthermore, each of L¹ to L³ independently represents a divalent aromatic hydrocarbon group having 6 to 12 carbon atoms, and X is an oxygen atom or a sulfur atom.

Another embodiment of the present invention is the light-emitting device having the above structure, in which the organic compound having a hole-transport property includes a plurality of alkyl groups.

Another embodiment of the present invention is the light-emitting device having the above structure, in which X is a sulfur atom.

Another embodiment of the present invention is the light-emitting device having the above structure, in which L¹ is any of groups represented by Structural Formulae (L-1) to (L-7) below.

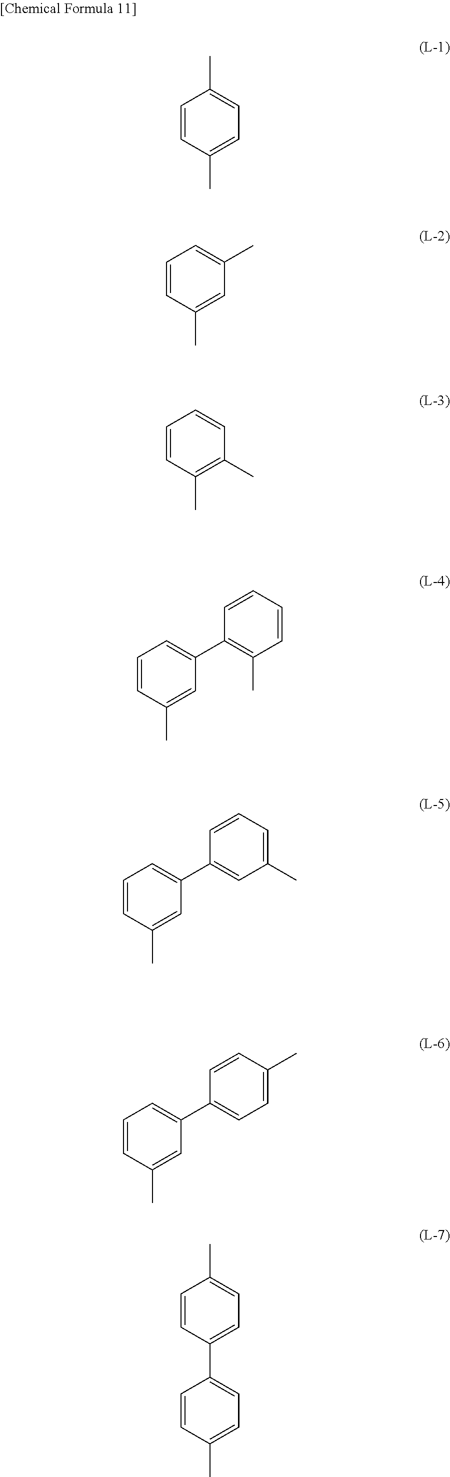

Another embodiment of the present invention is the light-emitting device having the above structure, in which L¹ is a group represented by Structural Formula (L-2) or (L-6) below.

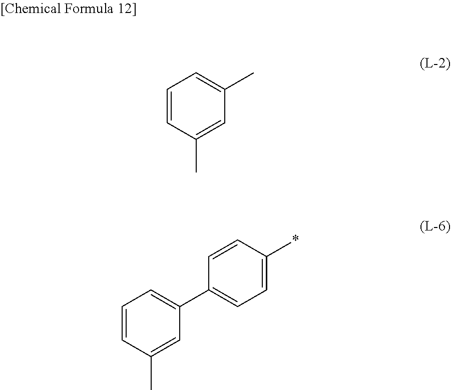

Note that (L-6) is bonded to a nitrogen atom at a position indicated by an asterisk.

Another embodiment of the present invention is the light-emitting device having the above structure, in which the Ar² is a group represented by General Formula (g3-1) or (g3-2) below.

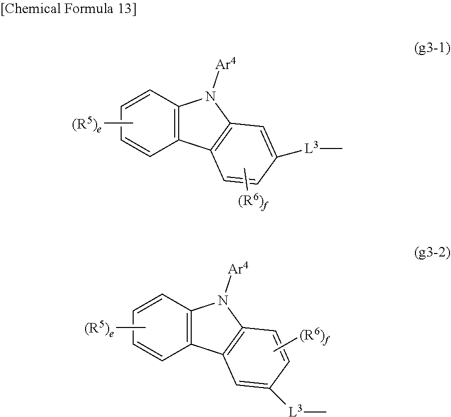

Note that in General Formula (g3-1) or (g3-2) above, each of R⁵ and R⁶ is independently any of a hydrocarbon group having 1 to 6 carbon atoms and an aromatic hydrocarbon group having 6 to 13 carbon atoms, and Ar⁴ is a substituted or unsubstituted phenyl group. Furthermore, e represents an integer of 0 to 4, and f represents an integer of 0 to 3. Furthermore, L³ represents a divalent aromatic hydrocarbon group having 6 to 18 carbon atoms.

Another embodiment of the present invention is the light-emitting device having the above structure, in which the Ar² is a group represented by (g3-1).

Another embodiment of the present invention is the light-emitting device having the above structure, in which the Ar³ is a group represented by General Formula (Ar³-1) or (Ar³-2) below.

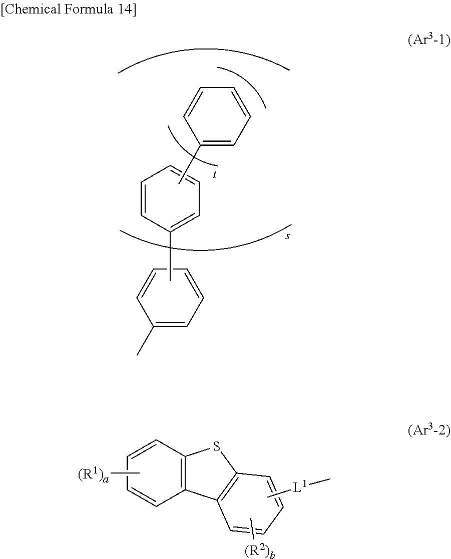

Note that in (Ar³-1), each of s and t is independently 0 or 1. In (Ar³-2), each of R¹ and R² is independently any of a hydrocarbon group having 1 to 6 carbon atoms and an aromatic hydrocarbon group having 6 to 13 carbon atoms, a represents an integer of 0 to 4, and b represents an integer of 0 to 3. Furthermore, L¹ represents a divalent aromatic hydrocarbon group having 6 to 12 carbon atoms.

Another embodiment of the present invention is the light-emitting device having the above structure, in which Ar³ is a group represented by General Formula (Ar³-2) above.

Another embodiment of the present invention is the light-emitting device having the above structure, in which the Ar³ is a group represented by General Formula (Ar³-1-1) or (Ar³-1-2) below.

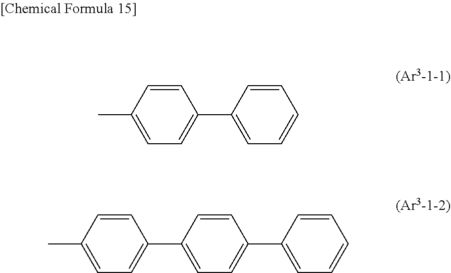

Another embodiment of the present invention is the light-emitting device having the above structure, in which the Ar³ is a group represented by General Formula (Ar³-1-1) below.

Another embodiment of the present invention is the light-emitting device having the above structure, in which the organic compound represented by General Formula (G1) is an organic compound represented by General Formula (G2) below.

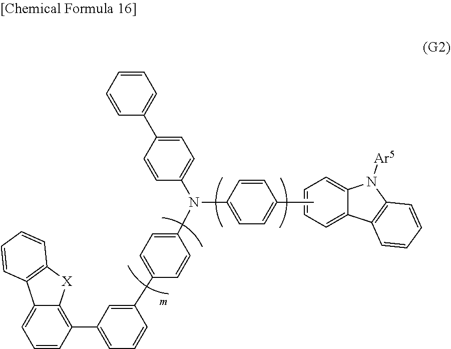

Note that in General Formula (G2) above, X is an oxygen atom or a sulfur atom, and Ar⁵ is a substituted or unsubstituted phenyl group. Furthermore, m is 0 or 1, and n represents an integer of 0 to 2.

Another embodiment of the present invention is the light-emitting device having the above structure, in which the second layer is positioned between the first layer and the light-emitting layer.

Another embodiment of the present invention is a light-emitting device material, which is used for the second layer in the above structure and is represented by General Formula (G1) above or General Formula (G2) above.

Another embodiment of the present invention is an organic compound represented by General Formula (G2) below.

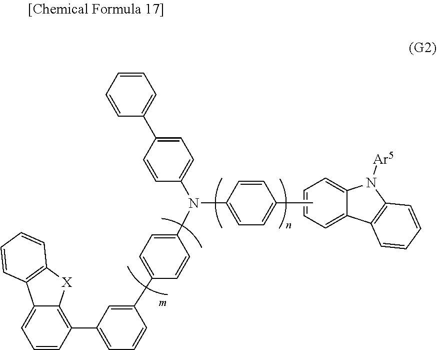

Note that in General Formula (G2) above, X is an oxygen atom or a sulfur atom, and Ar⁵ is a substituted or unsubstituted phenyl group. Furthermore, m is 0 or 1, and n represents an integer of 0 to 2.

Another embodiment of the present invention is the organic compound having the above structure, in which the X is a sulfur atom.

Another embodiment of the present invention is the organic compound having the above structure, in which n is 1.

Another embodiment of the present invention is an organic compound represented by Structural Formula (100) below.

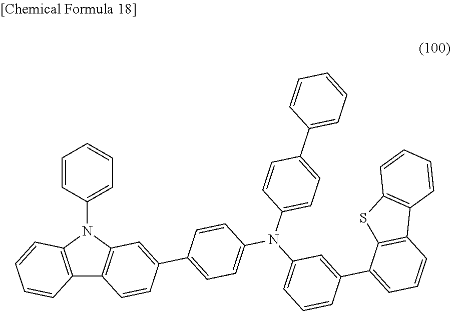 [SMILES: c1ccc(-c2ccc(N(c3ccc(-c4ccc5c6ccccc6n(-c6ccccc6)c5c4)cc3)c3cccc(-c4cccc5c4sc4ccccc45)c3)cc2)cc1]

Another embodiment of the present invention is an organic compound represented by Structural Formula (101) below.

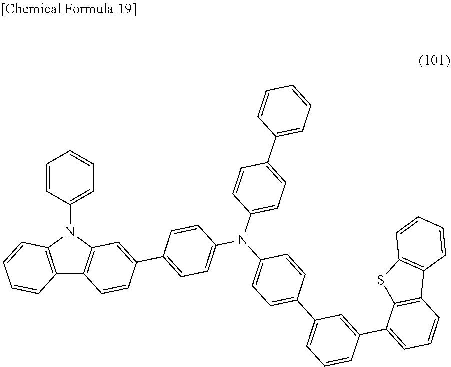 [SMILES: c1ccc(-c2ccc(N(c3ccc(-c4cccc(-c5cccc6c5sc5ccccc56)c4)cc3)c3ccc(-c4ccc5c6ccccc6n(-c6ccccc6)c5c4)cc3)cc2)cc1]

Another embodiment of the present invention is an organic compound represented by Structural Formula (104) below.

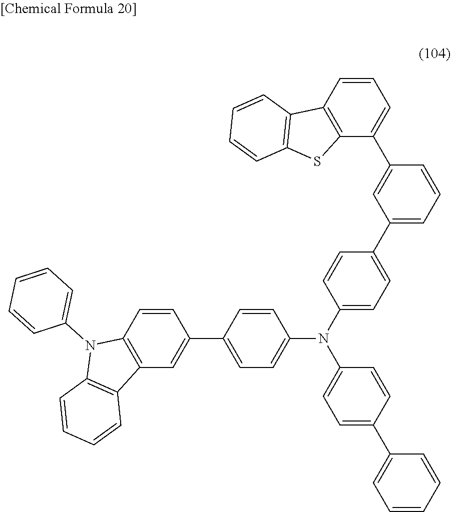 [SMILES: c1ccc(-c2ccc(N(c3ccc(-c4cccc(-c5cccc6c5sc5ccccc56)c4)cc3)c3ccc(-c4ccc5c(c4)c4ccccc4n5-c4ccccc4)cc3)cc2)cc1]

Another embodiment of the present invention is an organic compound represented by Structural Formula (103) below.

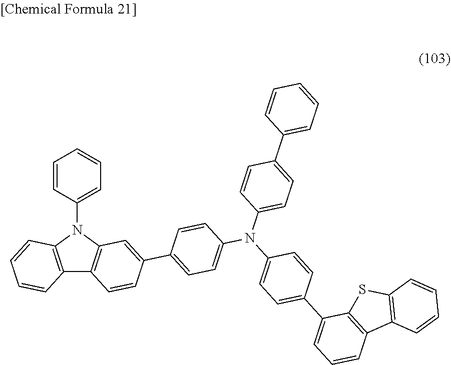 [SMILES: c1ccc(-c2ccc(N(c3ccc(-c4ccc5c6ccccc6n(-c6ccccc6)c5c4)cc3)c3ccc(-c4cccc5c4sc4ccccc45)cc3)cc2)cc1]

Another embodiment of the present invention is an electronic apparatus including the above electronic device or light-emitting device and a sensor, an operation button, a speaker, or a microphone.

Another embodiment of the present invention is a light-emitting apparatus including the above light-emitting device, and a transistor or a substrate.

Another embodiment of the present invention is a lighting device including the above light-emitting device and a housing.

Note that the light-emitting apparatus in this specification covers an image display device that uses a light-emitting device. The light-emitting apparatus may also include a module in which a light-emitting device is provided with a connector such as an anisotropic conductive film or a TCP (Tape Carrier Package), a module in which a printed wiring board is provided at the end of a TCP, or a module in which an IC (integrated circuit) is directly mounted on a light-emitting device by a COG (Chip On Glass) method. Furthermore, a lighting device or the like may include the light-emitting apparatus.

### Effect of the Invention

One embodiment of the present invention can provide a light-emitting device with high emission efficiency. One embodiment of the present invention can provide a light-emitting device with a low driving voltage. Another embodiment of the present invention can provide a light-emitting device with a long lifetime. One embodiment of the present invention can provide any of a light-emitting device, a light-emitting apparatus, an electronic apparatus, a display device, and an electronic device each having low power consumption. One embodiment of the present invention can provide any of a light-emitting device, a light-emitting apparatus, an electronic apparatus, a display device, and an electronic device each having favorable reliability.

Note that the description of these effects does not preclude the existence of other effects. One embodiment of the present invention does not need to have all these effects. Other effects will be apparent from and can be derived from the description of the specification, the drawings, the claims, and the like.

### BRIEF DESCRIPTION OF THE DRAWINGS

FIG. 1A, FIG. 1B, FIG. 1C, and FIG. 1D are schematic diagrams of light-emitting devices.

FIG. 2A and FIG. 2B are diagrams illustrating an active matrix light-emitting apparatus.

FIG. 3A and FIG. 3B are diagrams illustrating active matrix light-emitting apparatuses.

FIG. 4 is a diagram illustrating an active matrix light-emitting apparatus.

FIG. 5A and FIG. 5B are diagrams illustrating a passive matrix light-emitting apparatus.

FIG. 6A and FIG. 6B are diagrams illustrating a lighting device.

FIG. 7A, FIG. 7B1, FIG. 7B2, and FIG. 7C are diagrams illustrating electronic apparatuses.

FIG. 8A, FIG. 8B, and FIG. 8C are diagrams illustrating electronic apparatuses.

FIG. 9 is a diagram illustrating a lighting device.

FIG. 10 is a diagram illustrating lighting devices.

FIG. 11 is a diagram illustrating in-vehicle display devices and lighting devices.

FIG. 12A and FIG. 12B are diagrams illustrating an electronic apparatus.

FIG. 13A, FIG. 13B, and FIG. 13C are diagrams illustrating an electronic apparatus.

FIG. 14 shows luminance-current density characteristics of a light-emitting device 1, a light-emitting device 2, and a comparative light-emitting device 1 to a comparative light-emitting device 3.

FIG. 15 shows luminance-voltage characteristics of the light-emitting device 1, the light-emitting device 2, and the comparative light-emitting device 1 to the comparative light-emitting device 3.

FIG. 16 shows current efficiency-luminance characteristics of the light-emitting device 1, the light-emitting device 2, and the comparative light-emitting device 1 to the comparative light-emitting device 3.

FIG. 17 shows current-voltage characteristics of the light-emitting device 1, the light-emitting device 2, and the comparative light-emitting device 1 to the comparative light-emitting device 3.

FIG. 18 shows external quantum efficiency-luminance characteristics of the light-emitting device 1, the light-emitting device 2, and the comparative light-emitting device 1 to the comparative light-emitting device 3.

FIG. 19 shows power efficiency-luminance characteristics of the light-emitting device 1, the light-emitting device 2, and the comparative light-emitting device 1 to the comparative light-emitting device 3.

FIG. 20 shows emission spectra of the light-emitting device 1, the light-emitting device 2, and the comparative light-emitting device 1 to the comparative light-emitting device 3.

FIG. 21 shows normalized luminance-temporal change characteristics of the light-emitting device 1, the light-emitting device 2, and the comparative light-emitting device 1 to the comparative light-emitting device 3.

FIG. 22 shows current density-voltage characteristics of a device 3, a device 4, and a comparative device 4 to a comparative device 7.

FIG. 23 shows luminance-current density characteristics of a light-emitting device 5, a light-emitting device 6, and a comparative light-emitting device 8 to a comparative light-emitting device 11.

FIG. 24 shows luminance-voltage characteristics of the light-emitting device 5, the light-emitting device 6, and the comparative light-emitting device 8 to the comparative light-emitting device 11.

FIG. 25 shows current efficiency-luminance characteristics of the light-emitting device 5, the light-emitting device 6, and the comparative light-emitting device 8 to the comparative light-emitting device 11.

FIG. 26 shows current-voltage characteristics of the light-emitting device 5, the light-emitting device 6, and the comparative light-emitting device 8 to the comparative light-emitting device 11.

FIG. 27 shows external quantum efficiency-luminance characteristics of the light-emitting device 5, the light-emitting device 6, and the comparative light-emitting device 8 to the comparative light-emitting device 11.

FIG. 28 shows power efficiency-luminance characteristics of the light-emitting device 5, the light-emitting device 6, and the comparative light-emitting device 8 to the comparative light-emitting device 11.

FIG. 29 shows emission spectra of the light-emitting device 5, the light-emitting device 6, and the comparative light-emitting device 8 to the comparative light-emitting device 11.

FIG. 30 shows luminance-current density characteristics of a light-emitting device 7.

FIG. 31 shows luminance-voltage characteristics of the light-emitting device 7.

FIG. 32 shows current efficiency-luminance characteristics of the light-emitting device 7.

FIG. 33 shows current-voltage characteristics of the light-emitting device 7.

FIG. 34 shows external quantum efficiency-luminance characteristics of the light-emitting device 7.

FIG. 35 shows power efficiency-luminance characteristics of the light-emitting device 7.

FIG. 36 shows an emission spectrum of the light-emitting device 7.

FIG. 37 shows luminance-current density characteristics of a light-emitting device 8 and a comparative light-emitting device 12.

FIG. 38 shows luminance-voltage characteristics of the light-emitting device 8 and the comparative light-emitting device 12.

FIG. 39 shows current efficiency-luminance characteristics of the light-emitting device 8 and the comparative light-emitting device 12.

FIG. 40 shows current-voltage characteristics of the light-emitting device 8 and the comparative light-emitting device 12.

FIG. 41 shows BI-luminance characteristics of the light-emitting device 8 and the comparative light-emitting device 12.

FIG. 42 shows emission spectra of the light-emitting device 8 and the comparative light-emitting device 12.

FIG. 43 shows normalized luminance-temporal change characteristics of the light-emitting device 8 and the comparative light-emitting device 12.

FIG. 44A and FIG. 44B are 1H-NMR charts of PCBBiPDBt-02.

FIG. 45 shows an absorption spectrum and an emission spectrum of PCBBiPDBt-02 in a solution state.

FIG. 46 shows an absorption spectrum and an emission spectrum of PCBBiPDBt-02 in a thin film state.

FIG. 47 shows an MS spectrum of PCBBiPDBt-02.

FIG. 48A and FIG. 48B are 1H-NMR charts of mPCBBiPDBt-02.

FIG. 49 shows an absorption spectrum and an emission spectrum of mPCBBiPDBt-02 in a solution state.

FIG. 50 shows an absorption spectrum and an emission spectrum of mPCBBiPDBt-02 in a thin film state.

FIG. 51 shows MS spectrum of mPCBBiPDBt-02.

FIG. 52A and FIG. 52B are 1H-NMR charts of pmPCBBiBPDBt-02.

FIG. 53 shows an absorption spectrum and an emission spectrum of pmPCBBiBPDBt-02 in a solution state.

FIG. 54 shows an absorption spectrum and an emission spectrum of pmPCBBiBPDBt-02 in a thin film state.

FIG. 55 shows MS spectrum of pmPCBBiBPDBt-02.

FIG. 56A and FIG. 56B are 1H-NMR charts of pmPCBBiPDBt.

FIG. 57 shows an absorption spectrum and an emission spectrum of pmPCBBiPDBt in a solution state.

FIG. 58 shows an absorption spectrum and an emission spectrum of pmPCBBiPDBt in a thin film state.

FIG. 59A and FIG. 59B are 1H-NMR charts of pmPCBBiBPDBf-02.

FIG. 60 shows an absorption spectrum and an emission spectrum of pmPCBBiBPDBf-02 in a solution state.

FIG. 61 shows an absorption spectrum and an emission spectrum of pmPCBBiBPDBf-02 in a thin film state.

FIG. 62A and FIG. 62B are 1H-NMR charts of pmPCBiBPDBt-02.

FIG. 63 shows an absorption spectrum and an emission spectrum of pmPCBiBPDBt-02 in a solution state.

FIG. 64 shows an absorption spectrum and an emission spectrum of pmPCBiBPDBt-02 in a thin film state.

FIG. 65A and FIG. 65B are 1H-NMR charts of mmtBuBidFBi.

FIG. 66 shows an absorption spectrum and an emission spectrum of mmtBuBidFBi in a solution state.

### MODE FOR CARRYING OUT THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

### Embodiment 1

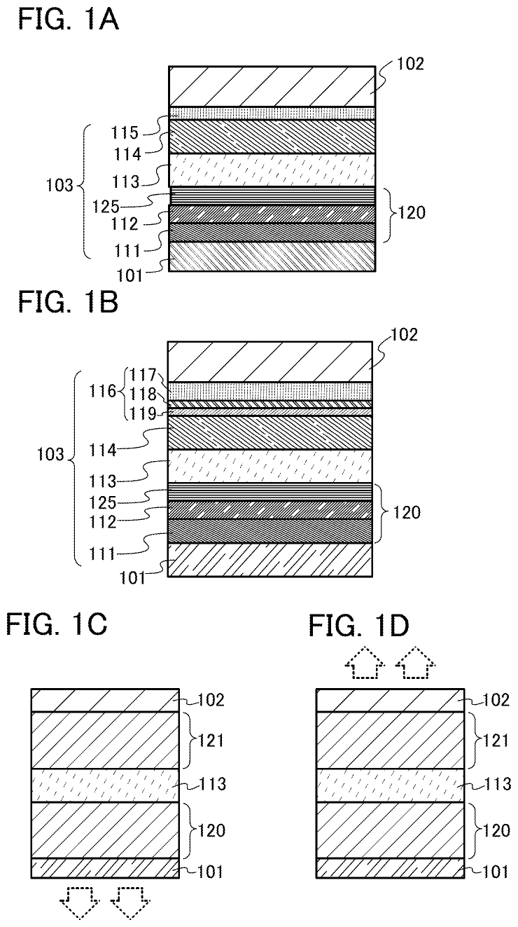

FIG. 1A is a diagram illustrating a light-emitting device of one embodiment of the present invention. FIG. 1 illustrates a structure including an anode 101, a cathode 102, and an EL layer 103, where the EL layer includes a hole-transport region 120, a light-emitting layer 113, an electron-transport layer 114, and an electron-injection layer 115. The light-emitting layer 113 is a layer containing at least a light-emitting material, and the hole-transport region 120 includes a hole-transport layer 112, a hole-injection layer 111, an electron-blocking layer 125, and the like. Note that the structure of the EL layer 103 is not limited to this structure, and an embodiment where some of the above-described layers are not formed or an embodiment where other functional layers such as a hole-blocking layer, an exciton-blocking layer, and an intermediate layer are formed may be employed.

One embodiment of the present invention has a structure provided with a low refractive index layer in a region between the light-emitting layer 113 and the anode 101 (the hole-transport region 120) in the EL layer 103.

The low refractive index layer is a layer-shaped region that is substantially parallel to the anode 101 or the cathode 102 and has a lower refractive index than at least the light-emitting layer 113. Since the refractive index of an organic compound included in a light-emitting device is typically approximately 1.8 to 1.9, the refractive index of the low refractive index layer is preferably lower than or equal to 1.75; specifically, the ordinary refractive index in the blue light emission region (greater than or equal to 455 nm and less than or equal to 465 nm) is preferably higher than or equal to 1.50 and lower than or equal to 1.75, or the ordinary refractive index with respect to light of 633 nm, which is typically used in refractive index measurement, is preferably higher than or equal to 1.45 and lower than or equal to 1.70. To form such a low refractive index layer, the low refractive index layer is formed using a material and a formation method with which the formed film has the above-described refractive index value.

Note that in the case where light is incident on a material having optical anisotropy, light with a plane of vibration parallel to the optical axis is referred to as extraordinary light (rays) and light with a plane of vibration perpendicular to the optical axis is referred to as ordinary light (rays); the refractive index of the material with respect to ordinary light might differ from that with respect to extraordinary light. In such a case, the ordinary refractive index and the extraordinary refractive index can be separately calculated by anisotropy analysis. Note that in the case where the measured material has both the ordinary refractive index and the extraordinary refractive index, the ordinary refractive index is used as an index in this specification. Note that when the simple term “refractive index” is used, the refractive index normally means the average of the ordinary refractive index and the extraordinary refractive index.

The entire region of the hole-transport region 120 is not necessarily the low refractive index layer, and at least part in the thickness direction of the hole-transport region 120 is provided as the low refractive index layer. For example, at least one of the functional layers provided in the hole-transport region 120, such as the hole-injection layer 111, the hole-transport layer 112, and an electron-blocking layer, is the low refractive index layer. The low refractive index layer may be a part of the functional layer. In other words, a structure may be employed in which the hole-transport layer 112 is composed of a plurality of layers and the refractive index of one of the layers is low.

The low refractive index layer can be formed by formation of each functional layer using a substance having a low refractive index as described above. However, in general, a high carrier-transport property and a low refractive index have a trade-off relationship. This is because the carrier-transport property of an organic compound largely depends on an unsaturated bond, and an organic compound having many unsaturated bonds tends to have a high refractive index. Even having a low refractive index, a material with a low carrier-transport property causes a problem such as a decrease in emission efficiency or reliability due to an increase in driving voltage or poor carrier balance, so that a light-emitting device with favorable characteristics cannot be obtained. Furthermore, even when a material has a sufficient carrier-transport property and a low refractive index, a light-emitting device with favorable reliability cannot be obtained if the material has a problem in the glass transition temperature (Tg) or the resistance due to an unstable structure.

A carrier-injection property at the interface between the low refractive index layer and another layer or the interface between low refractive index layers is also important. An organic compound having a hole-transport property with a low refractive index has a relatively high hole injection barrier even when having a sufficient hole mobility; thus, the use of the organic compound has resulted in an increase in driving voltage, in some cases. Even when the introduction of the low refractive index layer improves extraction efficiency and achieves a light-emitting device with high external quantum efficiency, the light-emitting device is disadvantageous in terms of energy efficiency and power efficiency when having high driving voltage; thus, an effect of reducing power consumption is not as significant as expected, in some cases.

Thus, one embodiment of the present invention discloses a structure in which a layer containing an organic compound having a specific structure is provided in contact with the low refractive index layer so that a carrier injection barrier of the organic compound having a hole-transport property with a low refractive index can be reduced and an increase in driving voltage due to the use of the low refractive index layer can be inhibited. Note that the layer containing the organic compound is preferably provided between the low refractive index layer and the light-emitting layer.

The organic compound having a specific structure that can inhibit an increase in driving voltage is a first organic compound having an arylamine structure, in which a first group, a second group, and a third group are bonded to a nitrogen atom. Here, the first group is a group including a carbazole structure, the second group is a group including a dibenzofuran structure or a dibenzothiophene structure, and the third group is a group including an aromatic hydrocarbon structure having 6 to 18 carbon atoms or a heteroaromatic hydrocarbon structure having 4 to 26 carbon atoms.

When a layer containing the first organic compound having such a structure is provided in contact with the layer containing the organic compound having a hole-transport property with a low refractive index, driving voltage can be specifically improved. When such a stacked-layer structure is provided in the hole-transport region 120, an increase in driving voltage can be inhibited and a light-emitting device with extremely favorable external quantum efficiency, power efficiency, and energy efficiency can be obtained.

Note that the carbazole structure included in the first group of the first organic compound preferably has a bonding site at any of the 2-position, the 3-position, and the 9-position, and is preferably bonded to the above nitrogen of amine at the bonding site directly or through a divalent aromatic hydrocarbon group. It is particularly preferred that the bonding site be at the 2-position or the 3-position the bond be at the 2-position or the 3-position, and further preferred that the bonding site be at the 2-position and the bond be at the 2-position. Furthermore, it is preferred that the divalent aromatic hydrocarbon group be a substituted or unsubstituted phenylene group or a substituted or unsubstituted biphenyldiyl group, it is further preferred that they be each an unsubstituted group, and it is still further preferred that an unsubstituted p-phenylene group be used.

The dibenzofuran structure or the dibenzothiophene structure included in the second group of the first organic compound is preferably bonded to the above nitrogen of amine through a divalent aromatic hydrocarbon group. It is preferred that the divalent aromatic hydrocarbon group be a substituted or unsubstituted phenylene group or a substituted or unsubstituted biphenyldiyl group, and it is further preferred that they be each an unsubstituted group. Note that it is still further preferred that a bonding position of either one of the phenylene group or a benzene ring in the biphenyldiyl group included in the second group be the meta-position.

In the case where the third group of the first organic compound has an aromatic hydrocarbon structure having 6 to 18 carbon atoms, it is preferred that the aromatic hydrocarbon group be a substituted or unsubstituted phenyl group, a substituted or unsubstituted biphenyl group, or a substituted or unsubstituted terphenyl group, and it is further preferred that they be each an unsubstituted group. Furthermore, in the case where the third group is a biphenyl group or a terphenyl group, all benzene rings in the group preferably have bonds at the para-position.

In the case where the third group of the first organic compound is a group including a heteroaromatic hydrocarbon structure having 4 to 26 carbon atoms, the heteroaromatic hydrocarbon structure is preferably a substituted or unsubstituted dibenzofuran structure or a substituted or unsubstituted dibenzothiophene structure, and particularly preferably a dibenzothiophene structure.

A preferred example of the first organic compound can be represented by General Formula (G1) below.

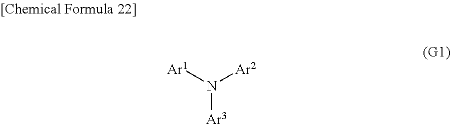

Note that in General Formula (G1) above, Ar¹ is a group represented by General Formula (g1) below, Ar² is a group represented by General Formula (g2) or (g3) below, and Ar³ is any of a group represented by General Formula (g1) below and an aromatic hydrocarbon group having 6 to 18 carbon atoms.

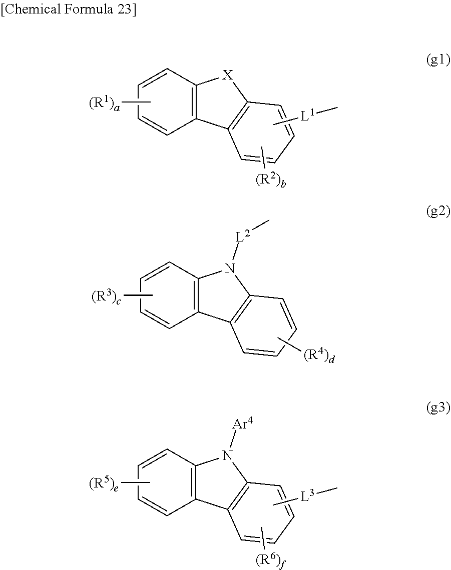

Note that in General Formulae (g1) to (g3) above, each of R¹ to R⁶ is independently any of a hydrocarbon group having 1 to 6 carbon atoms and an aromatic hydrocarbon group having 6 to 13 carbon atoms, and Ar⁴ is a substituted or unsubstituted phenyl group. Furthermore, each of a, c, d, and e independently represents an integer of 0 to 4, and each of b and f independently represents an integer of 0 to 3. Each of L¹ to L³ independently represents a substituted or unsubstituted divalent aromatic hydrocarbon group having 6 to 12 carbon atoms, and X is an oxygen atom or a sulfur atom.

Note that it is particularly preferred that L¹ in the group represented by General Formula (g1) be any of groups represented by Structural Formula (L-1) to Structural Formula (L-7) below.

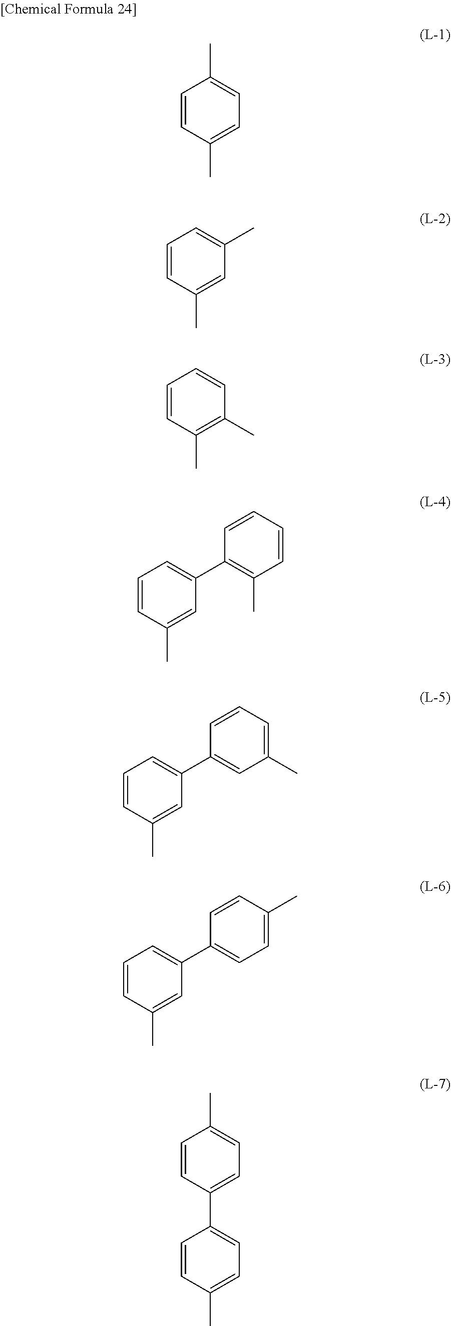

It is further preferred that L¹ be a group represented by Structural Formula (L-1) or (L-6) below. Note that it is still further preferred that, in the group represented by Structural Formula (L-6) below, a nitrogen atom be bonded at a position indicated by an asterisk.

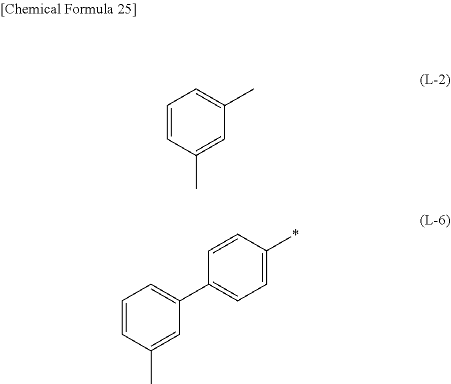

In General Formula (G1) above, Ar² is preferably the group represented by General Formula (g3) above, particularly preferably a group represented by General Formula (g3-1) or General Formula (g3-2) below, and further preferably the group represented by General Formula (g3-1) below.

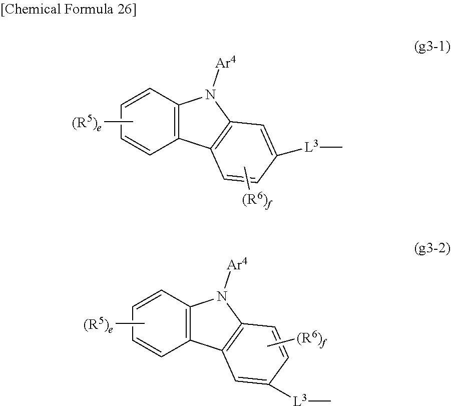

Note that in General Formula (g3-1) or (g3-2) above, each of R⁵ and R⁶ is independently any of a hydrocarbon group having 1 to 6 carbon atoms and an aromatic hydrocarbon group having 6 to 13 carbon atoms, and Ar⁴ is a substituted or unsubstituted phenyl group. Furthermore, e represents an integer of 0 to 4, and f represents an integer of 0 to 3. Furthermore, L³ represents a divalent aromatic hydrocarbon group having 6 to 18 carbon atoms.

In the organic compound represented by General Formula (G1) above, Ar³ is preferably a group represented by General Formula (Ar³-1) below, or a group represented by (Ar³-2).

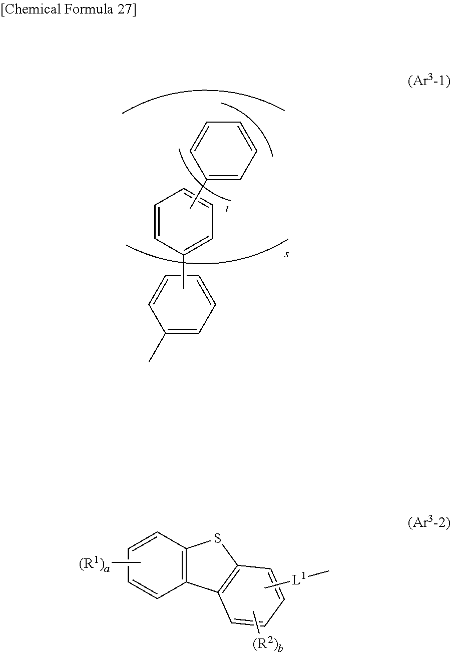

Note that in (Ar³-1), each of s and t is independently 0 or 1. In (Ar³-2), each of R¹ and R² is independently any of a hydrocarbon group having 1 to 6 carbon atoms and an aromatic hydrocarbon group having 6 to 13 carbon atoms, a represents an integer of 0 to 4, and b represents an integer of 0 to 3. Furthermore, L¹ represents a divalent aromatic hydrocarbon group having 6 to 12 carbon atoms.

Furthermore, (Ar³-1) is preferably a group represented by Structural Formula (Ar³-1-1) or Structural Formula (Ar³-1-2) below, and particularly preferably (Ar³-1-1).

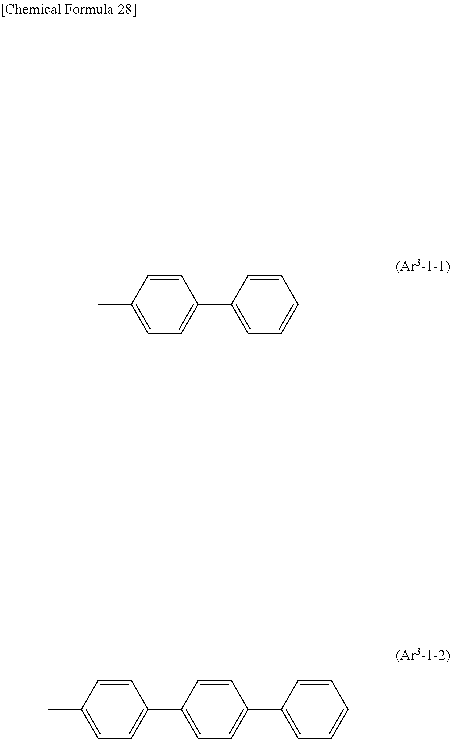

Note that in the organic compound represented by General Formula (G1) above, X is preferably a sulfur atom.

Specific examples of R¹ to R⁶ in the organic compound represented by General Formula (G1) above include a methyl group, an ethyl group, a propyl group, an isopropyl group, a butyl group, a tert-butyl group, a pentyl group, a hexyl group, a phenyl group, a biphenyl group, a naphthyl group, a fluorenyl group, and the like. In the case where R¹ to R⁶ are each a plurality of groups, the plurality of groups may be the same or different from one another. In the case where two alkyl groups are bonded to adjacent carbon atoms, the alkyl groups may be bonded to each other to form a ring.

The organic compound represented by General Formula (G1) above is particularly preferably an organic compound represented by General Formula (G2) below because in that case, a light-emitting device with further favorable driving voltage can be obtained.

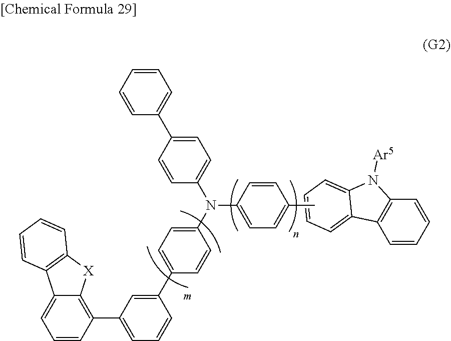

Note that in General Formula (G2) above, X is an oxygen atom or a sulfur atom, and Ar⁵ is a substituted or unsubstituted phenyl group. Furthermore, m is 0 or 1, and n represents an integer of 0 to 2. Note that in the organic compound represented by General Formula (G2) above, X is preferably a sulfur atom and n is preferably 1.

When the expression “substituted or unsubstituted” is used in the above description, the substituent is an alkyl group having 1 to 4 carbon atoms, specifically a methyl group, an ethyl group, a propyl group, an isopropyl group, a butyl group, a tert-butyl group, or the like.

Specific examples of the organic compound represented by General Formula (G2) above are shown below. Note that these are merely examples.

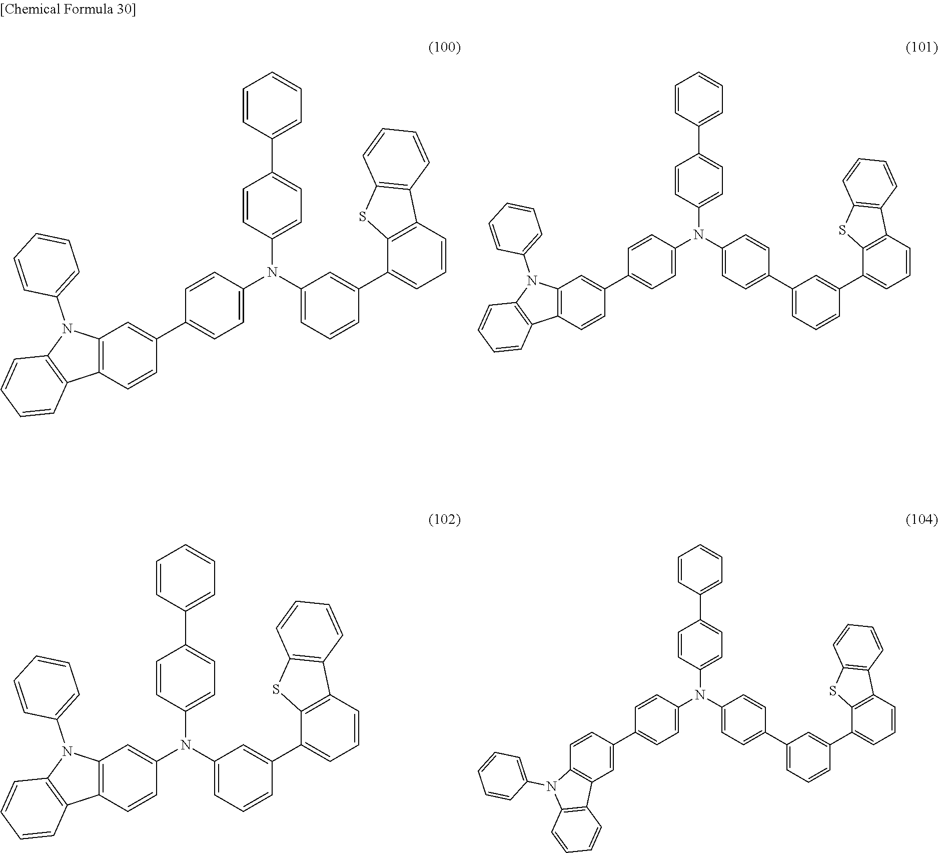

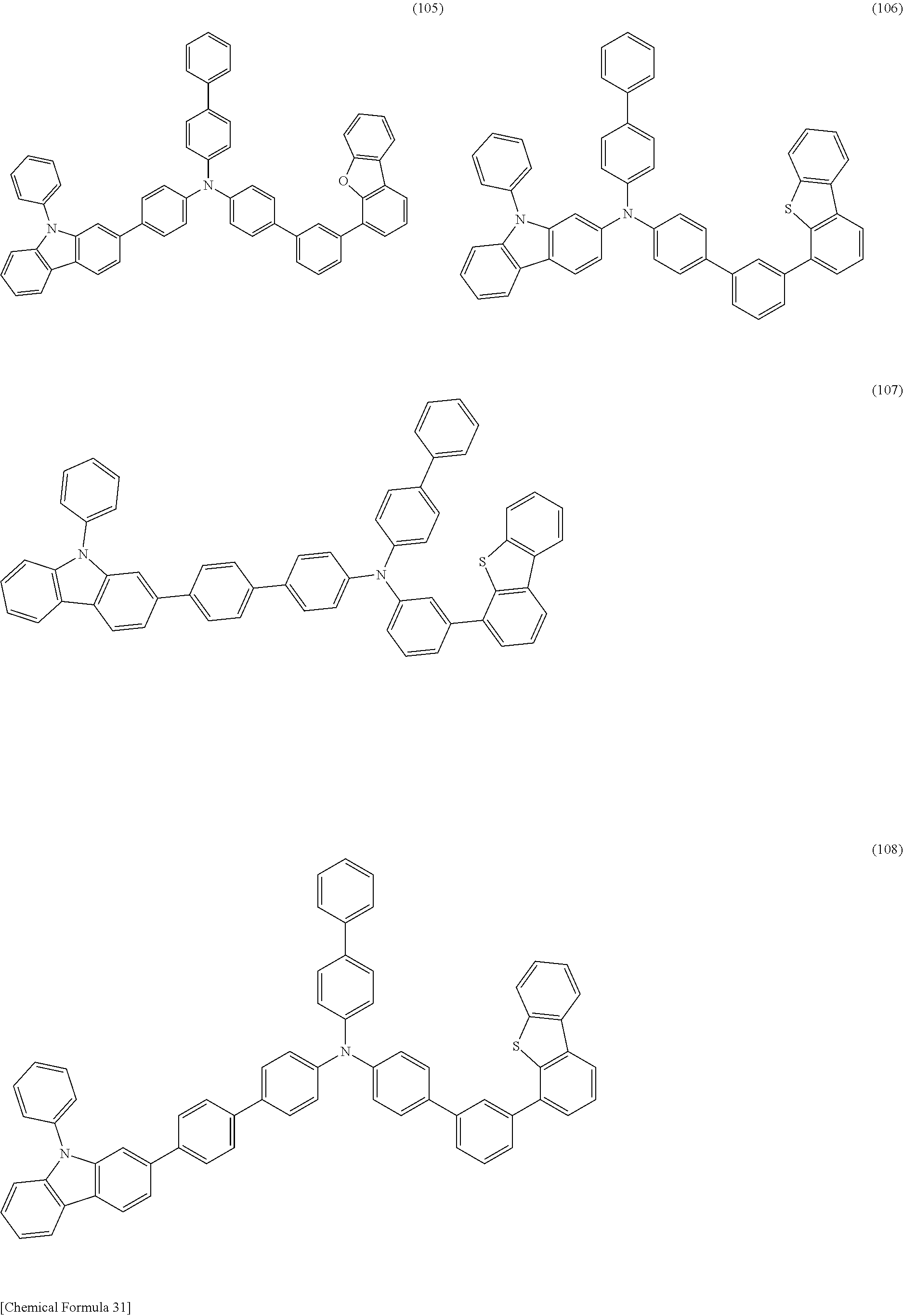

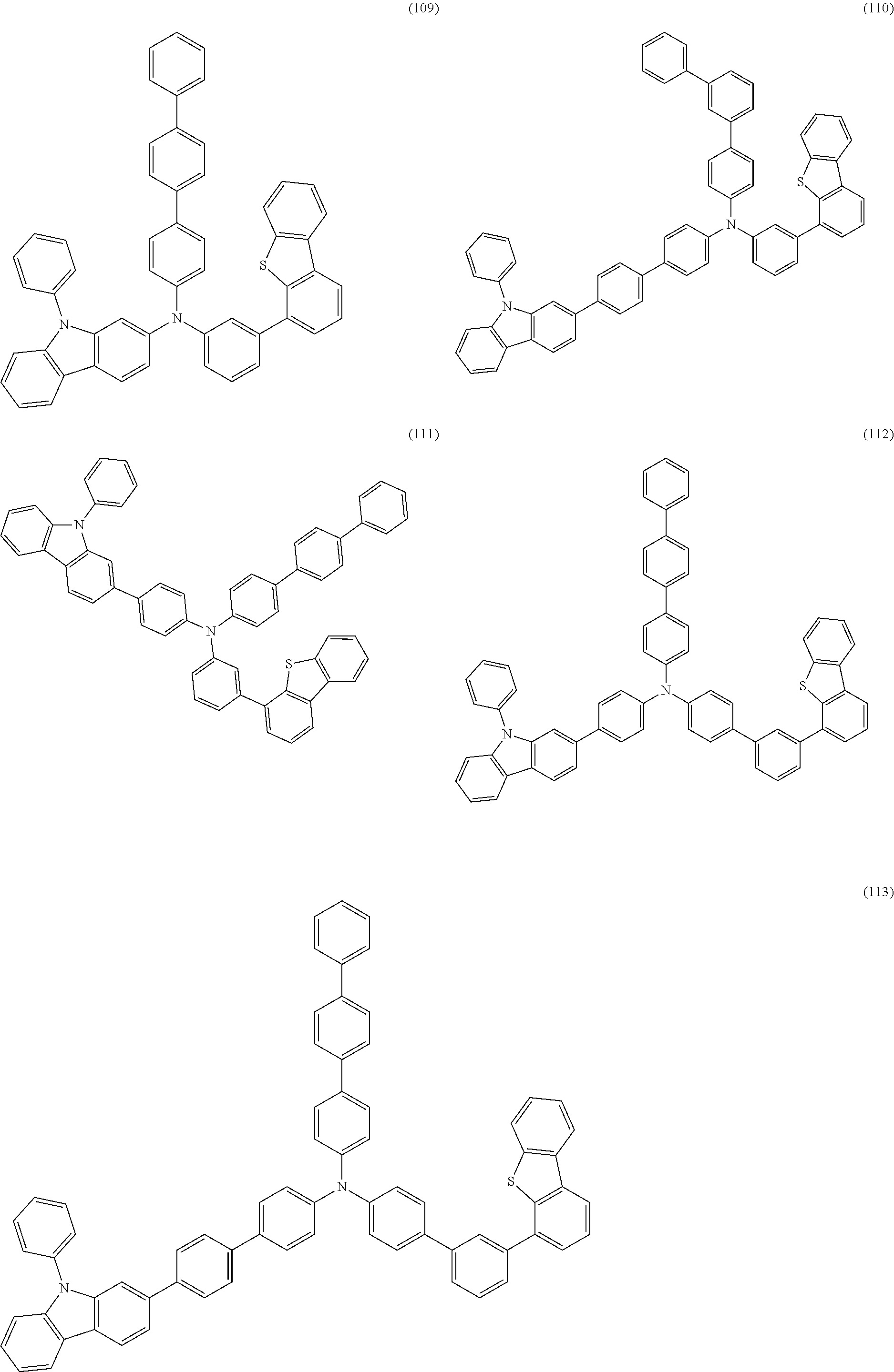

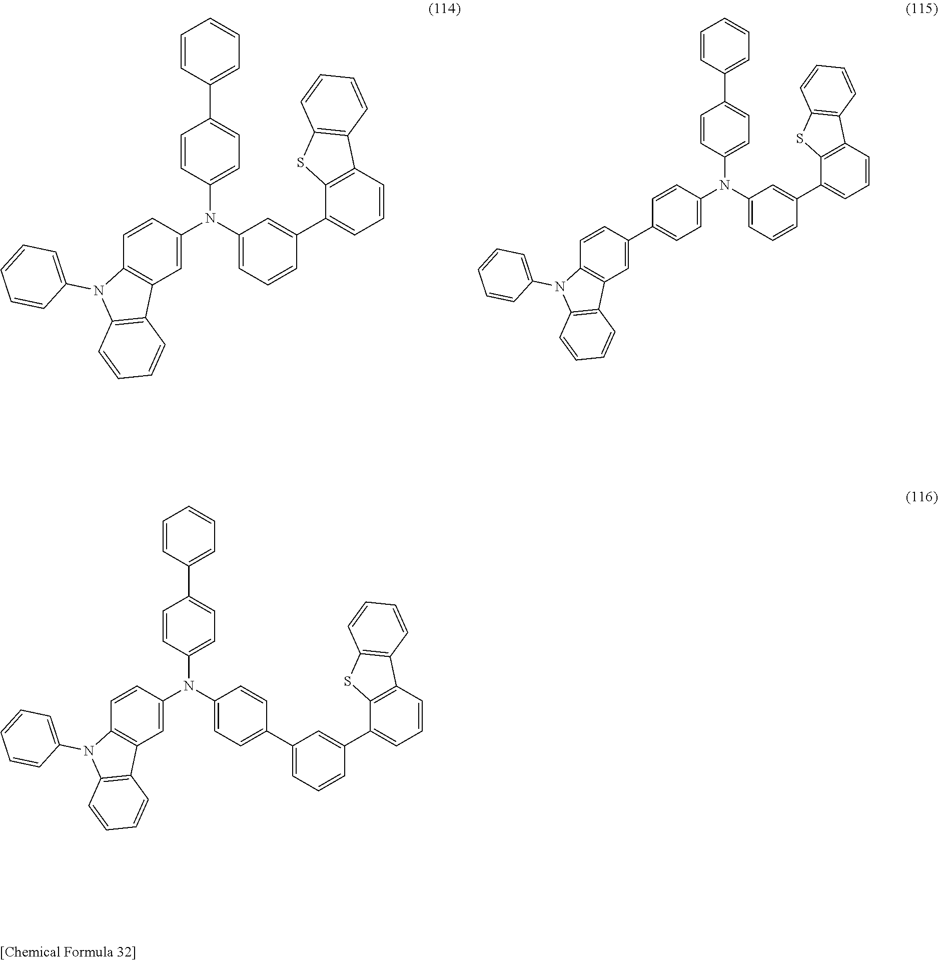

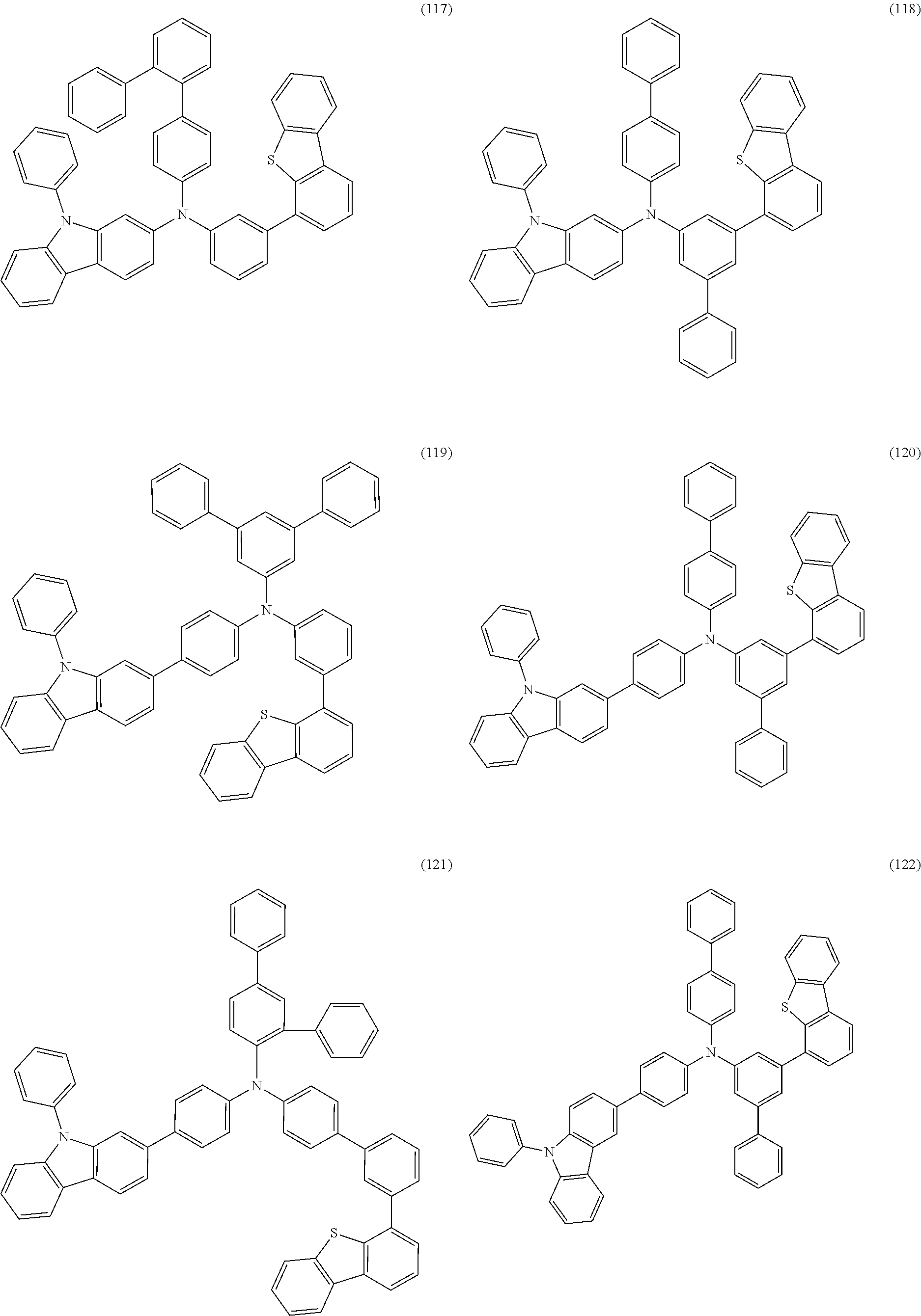

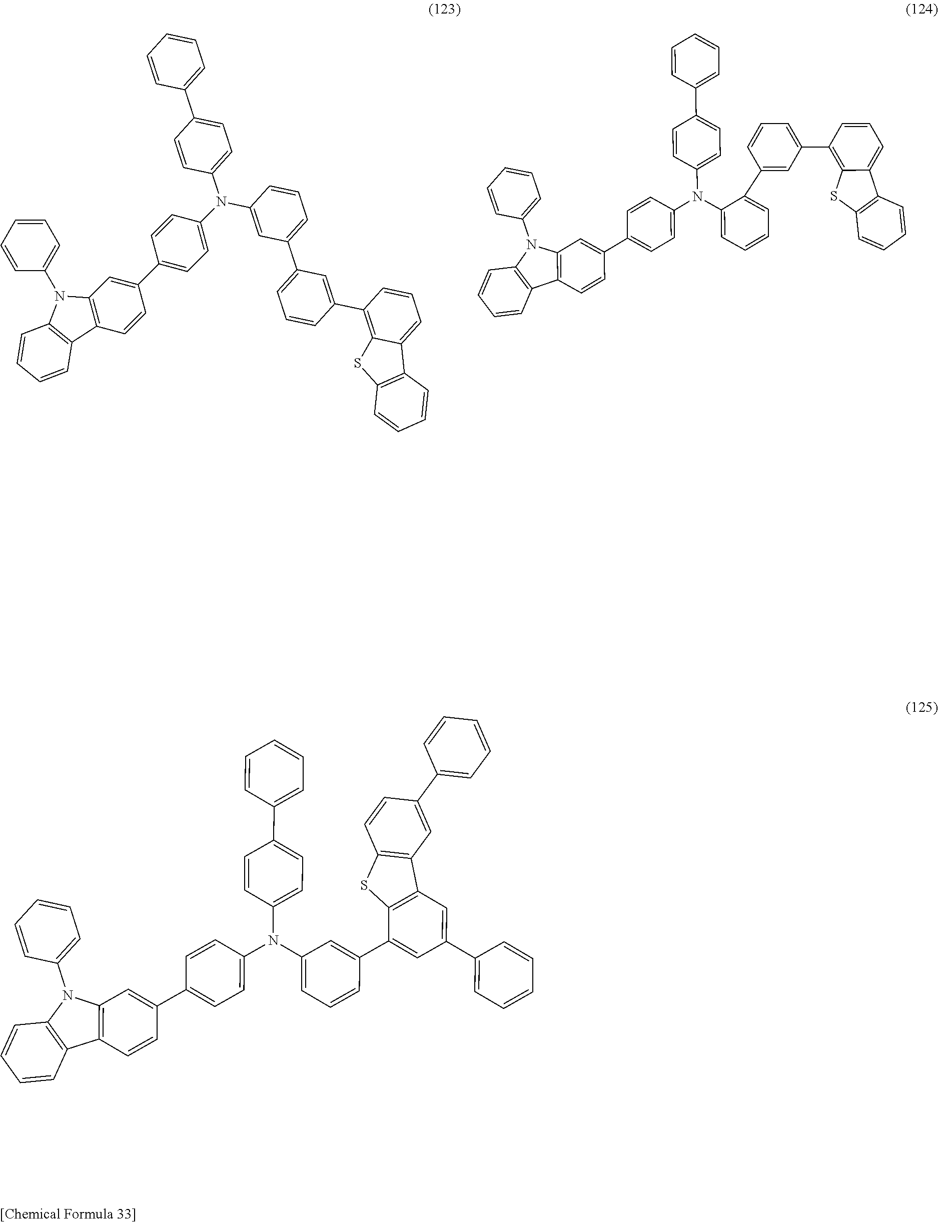

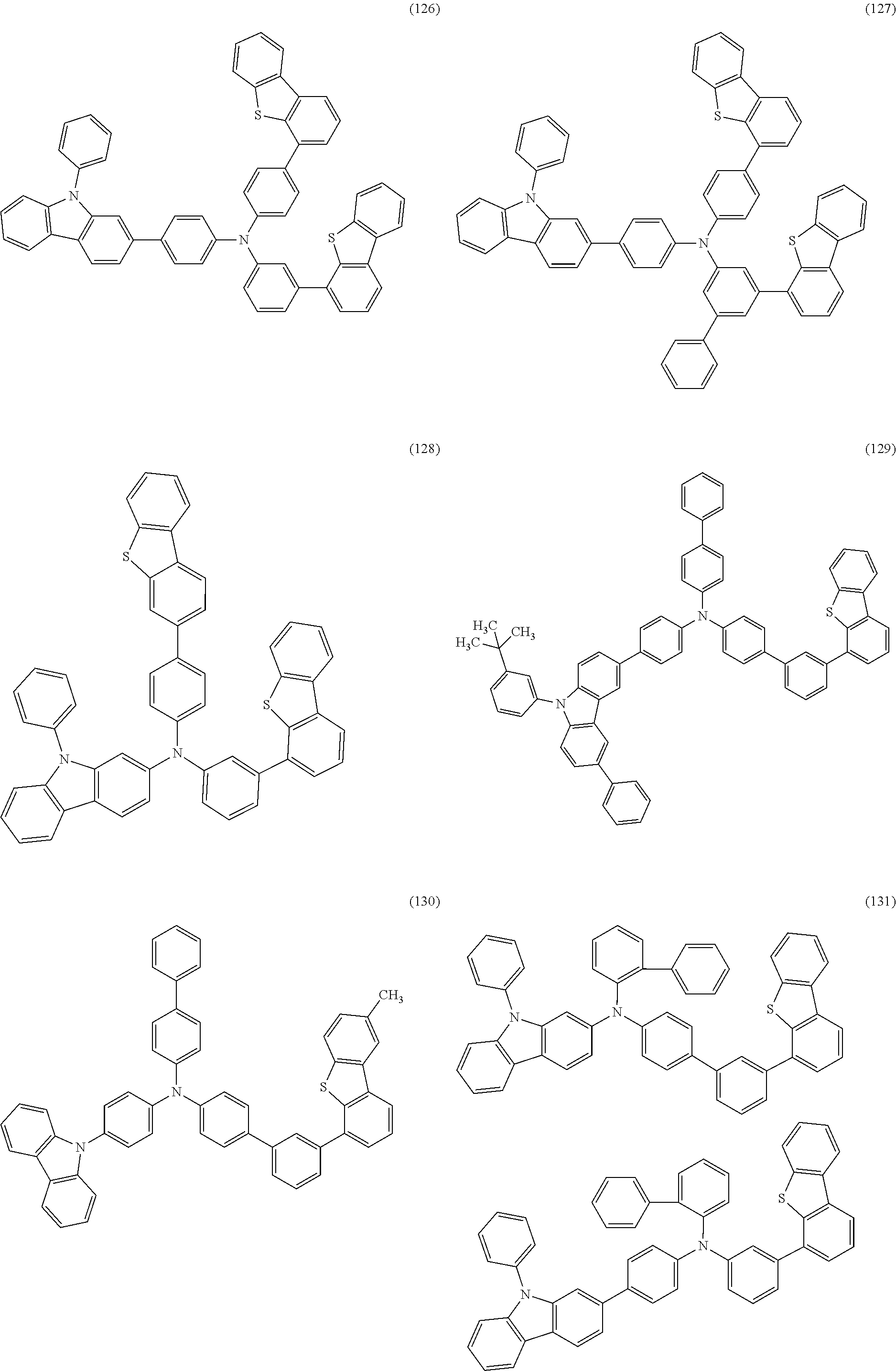

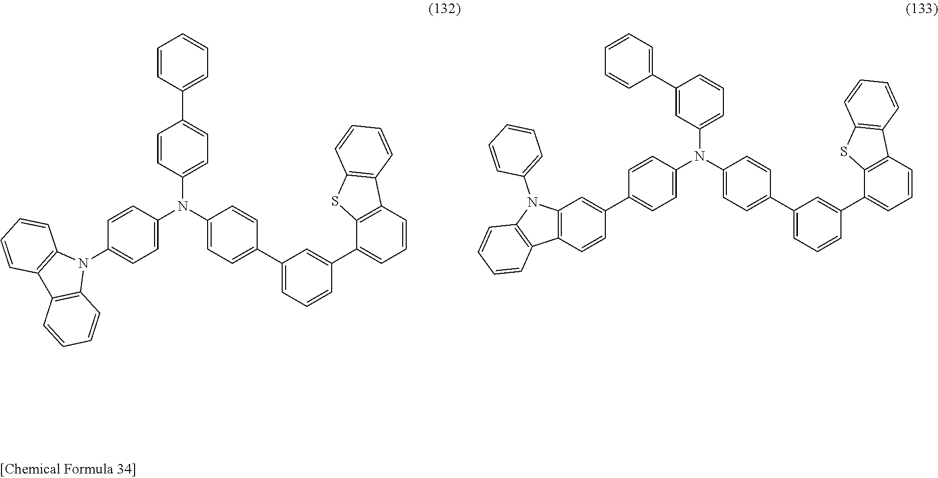

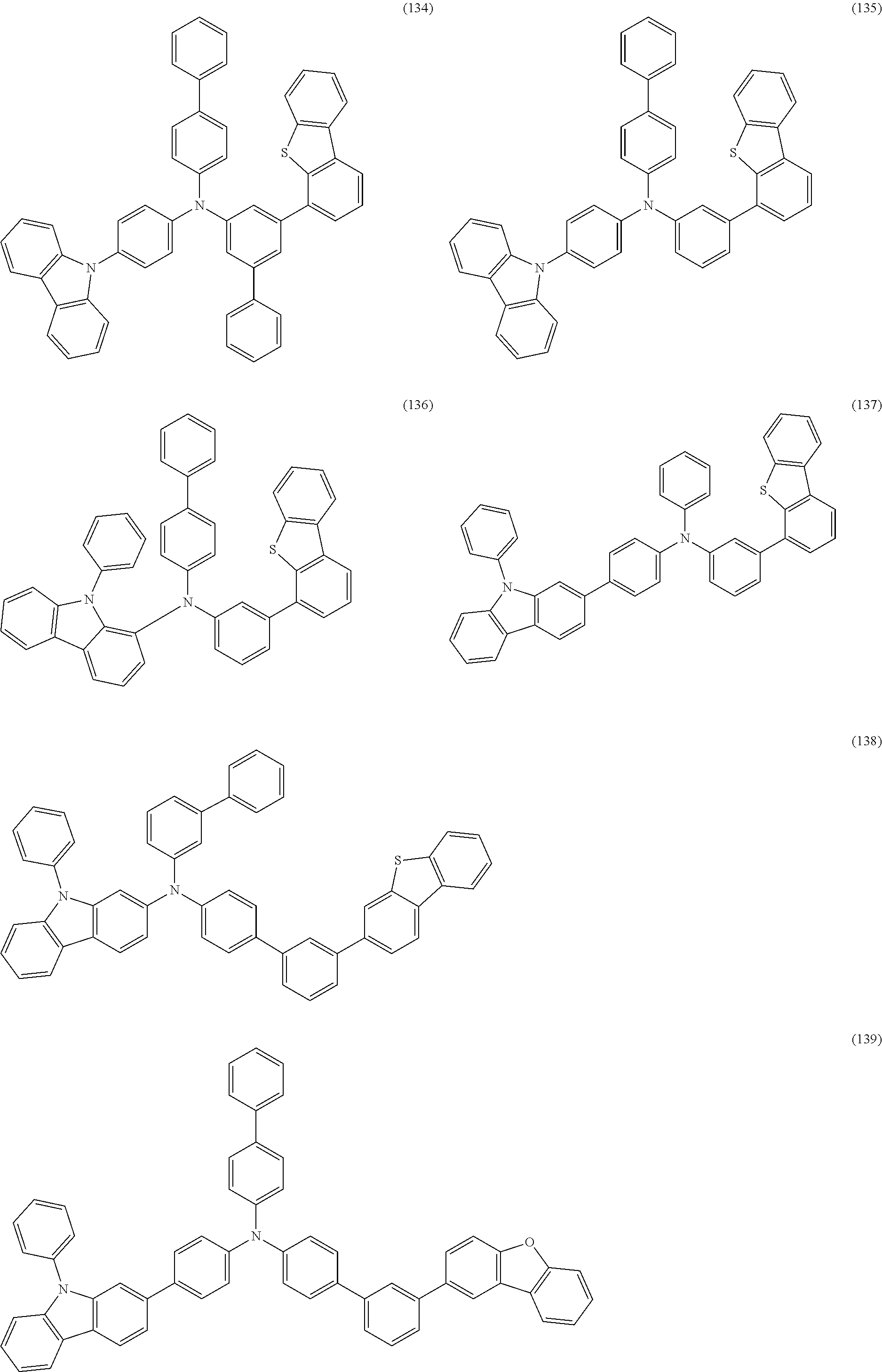

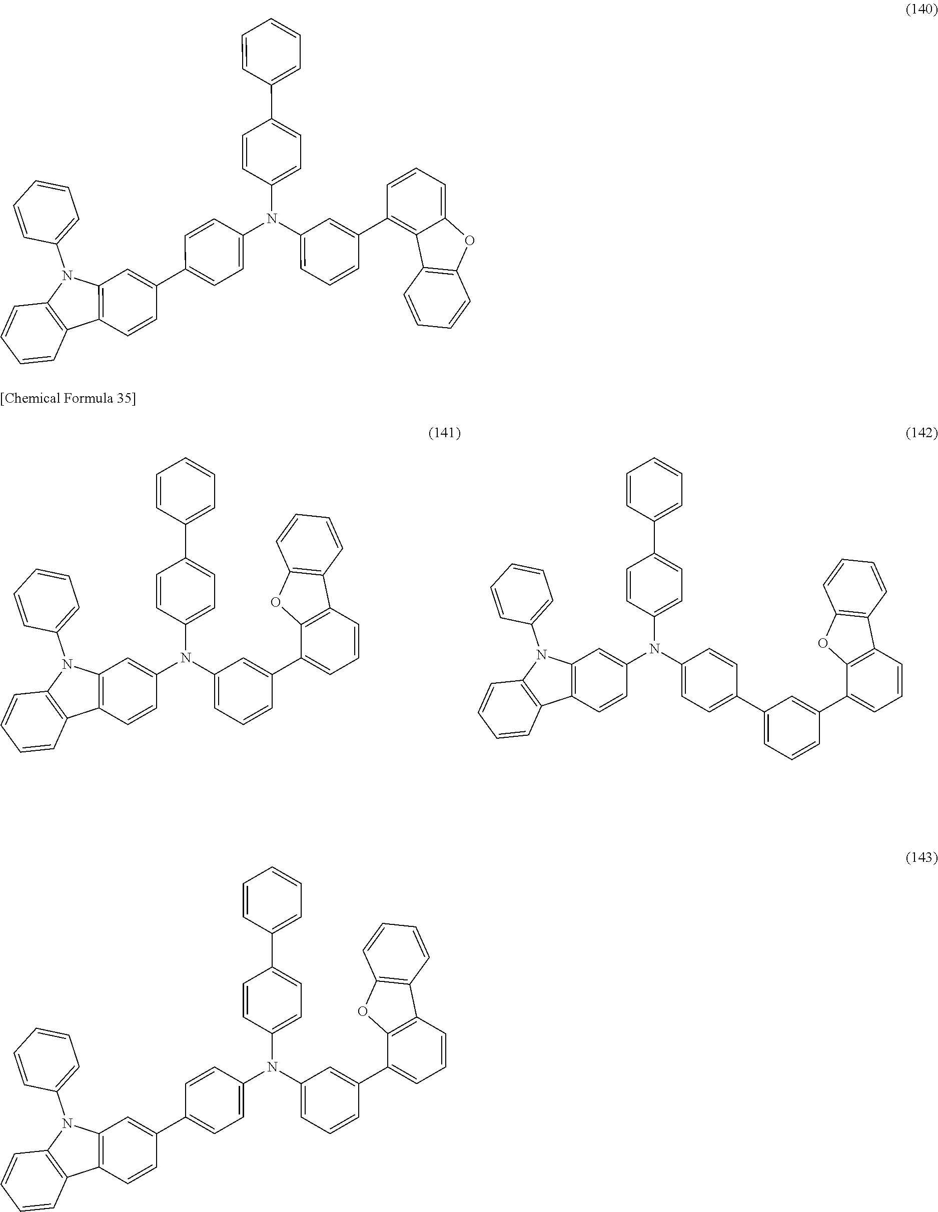

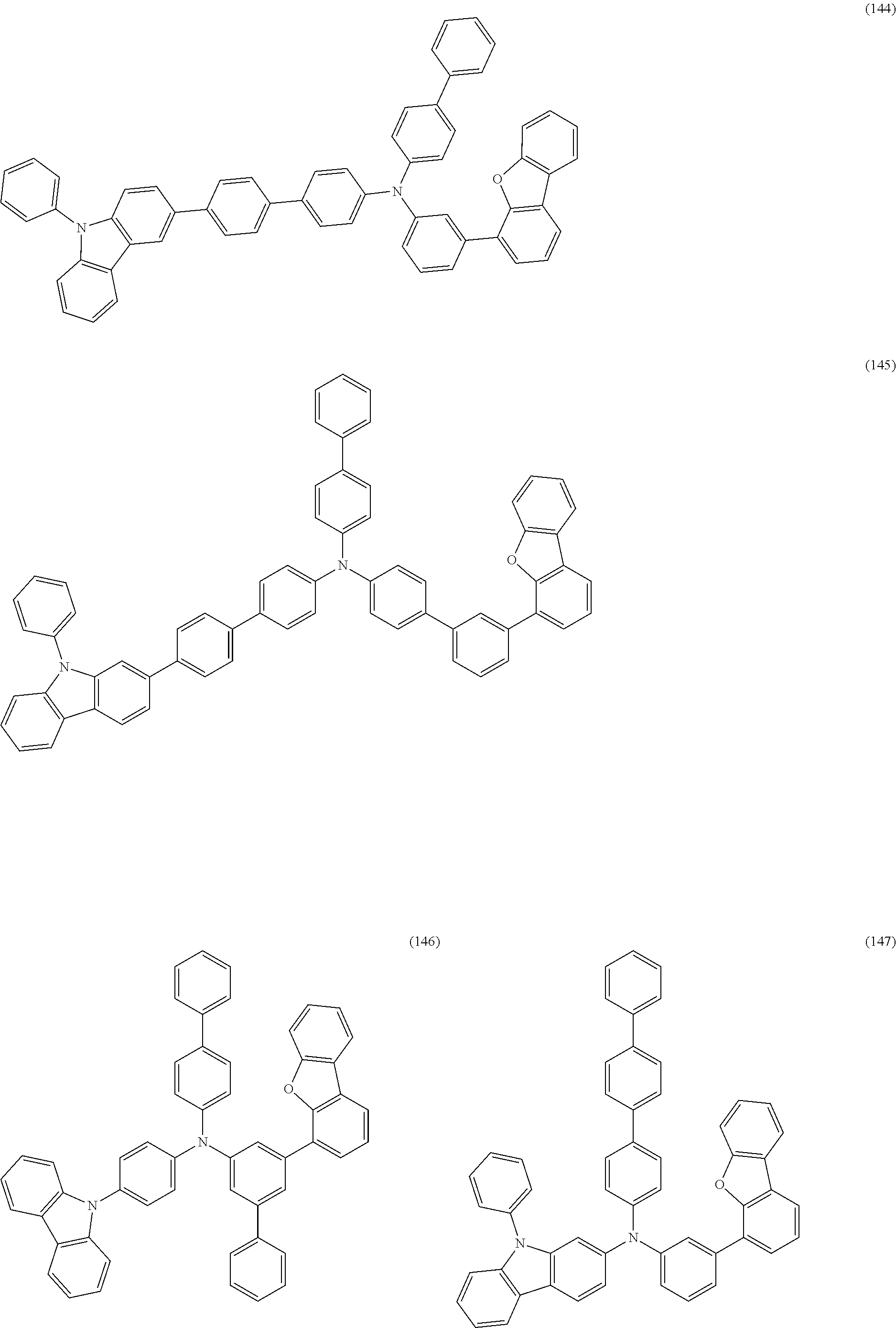

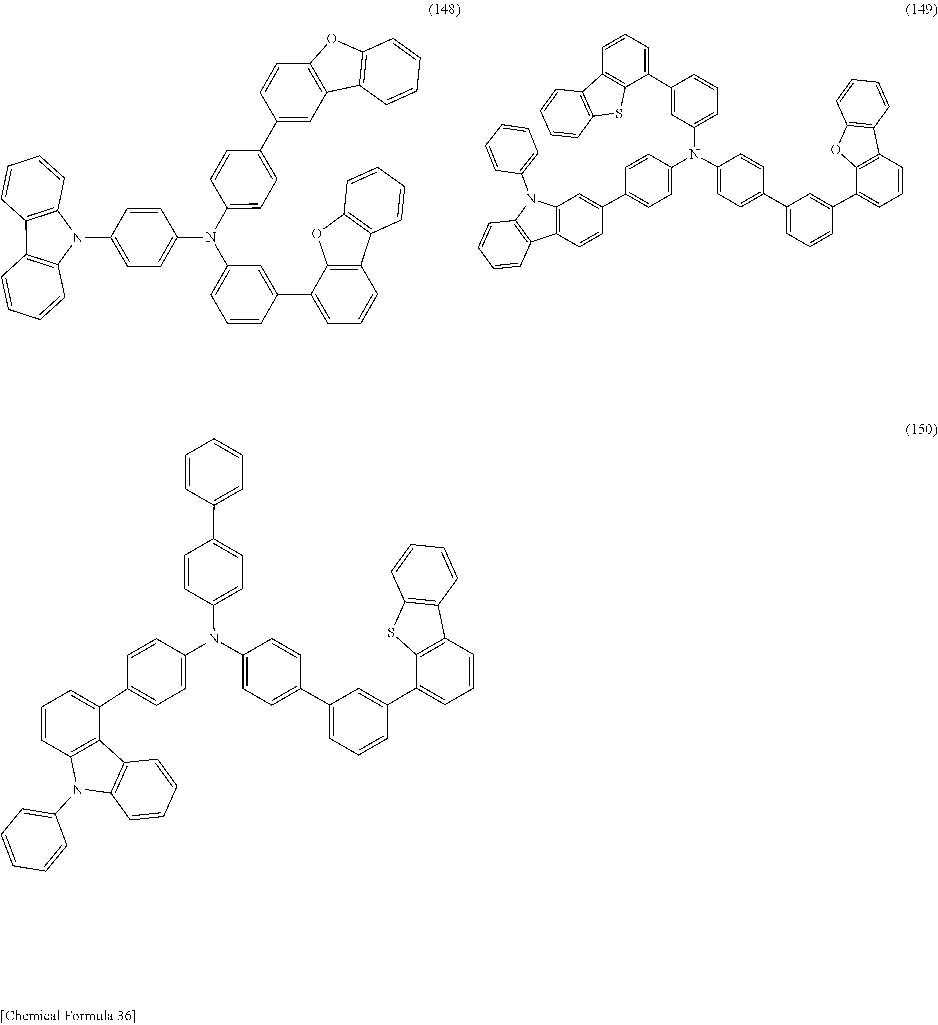

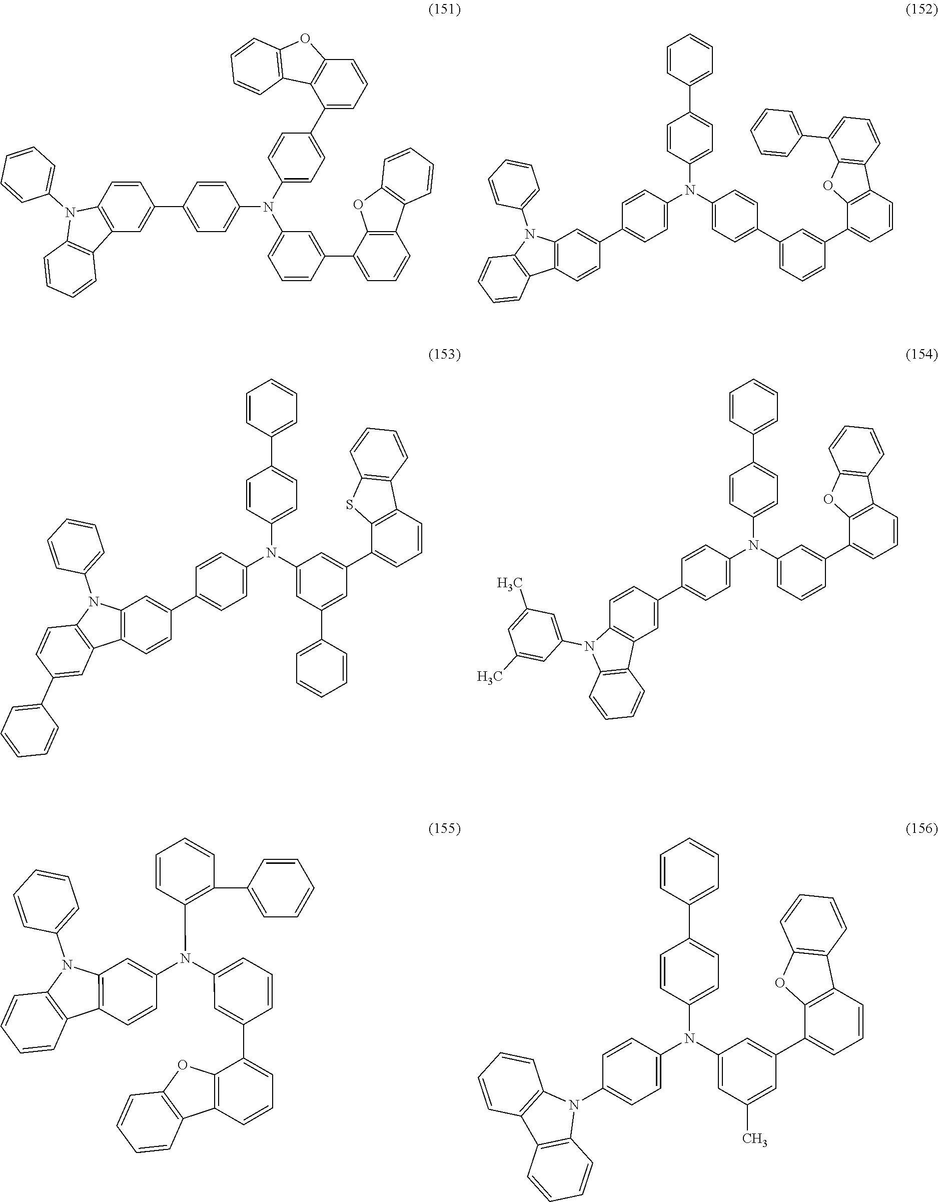

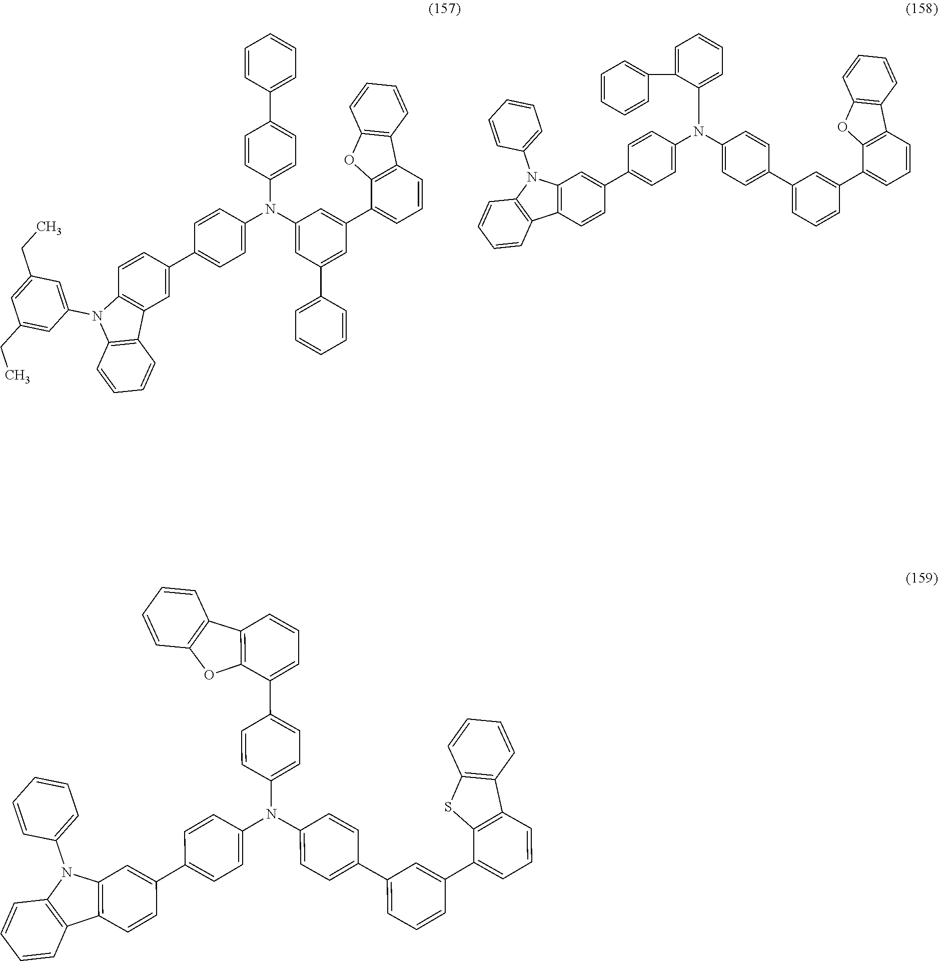

Note that the layer containing the first organic compound is provided as one of the functional layers included in the hole-transport region 120. The first organic compound has an effective LUMO level when used for an electron-blocking layer; thus, the layer containing the first organic compound is preferably provided between the hole-transport layer 112 and the light-emitting layer 113 as an electron-blocking layer. Each of the functional layers may include a plurality of layers and the layer containing the first organic compound may be provided as one of the layers. For example, the hole-transport layer can include a plurality of layers and one of the layers can be the layer containing the first organic compound.

As described above, it is extremely difficult for the organic compound having a hole-transport property with a low refractive index contained in the low refractive index layer to have all of a high carrier-transport property, a low refractive index, and favorable durability. Accordingly, in the light-emitting device of one embodiment of the present invention, a second organic compound having a structure described below is preferably used as a material contained in the low refractive index layer.

As the second organic compound having a hole-transport property, an organic compound including a first aromatic group, a second aromatic group, and a third aromatic group, in which the first aromatic group, the second aromatic group, and the third aromatic group are bonded to the same nitrogen atom, is preferably used.

In the second organic compound, the proportion of carbon atoms forming a bond by the sp3 hybrid orbitals to the total number of carbon atoms in the molecule is preferably higher than or equal to 23% and lower than or equal to 55%. In addition, it is preferable that the integral value of signals at lower than 4 ppm exceed the integral value of signals at 4 ppm or higher in the results of ¹H-NMR measurement conducted on the monoamine compound.

The second organic compound preferably has at least one fluorene skeleton. One or more of the first aromatic group, the second aromatic group, and the third aromatic group are preferably a fluorene skeleton.

Examples of the above-described second organic compound having a hole-transport property include organic compounds having structures represented by General Formulae (Gₕ₁1) to (Gₕ₁4) below.

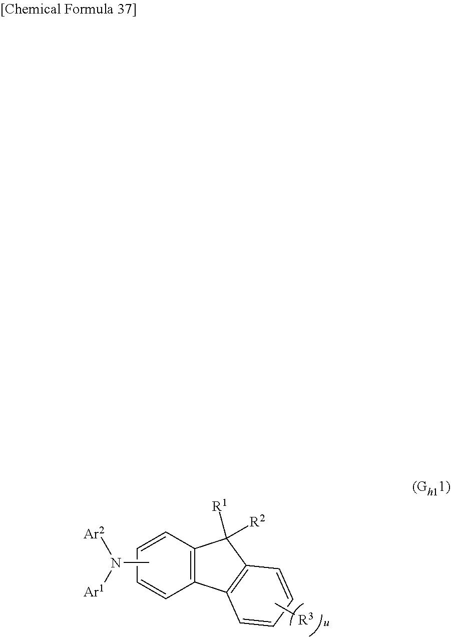

In General Formula (Gₕ₁1), each of Ar¹ and Ar² independently represents a benzene ring or a substituent in which two or three benzene rings are bonded to each other. Note that one or both of Ar¹ and Ar² have one or more hydrocarbon groups each having 1 to 12 carbon atoms forming a bond only by the sp3 hybrid orbitals. The total number of carbon atoms contained in all of the hydrocarbon groups bonded to Ar¹ and Ar² is 8 or more and the total number of carbon atoms contained in all of the hydrocarbon groups bonded to either Ar¹ or Ar² is 6 or more. Note that in the case where a plurality of straight-chain alkyl groups each having one or two carbon atoms are bonded to Ar¹ or Ar² as the hydrocarbon groups, the straight-chain alkyl groups may be bonded to each other to form a ring.

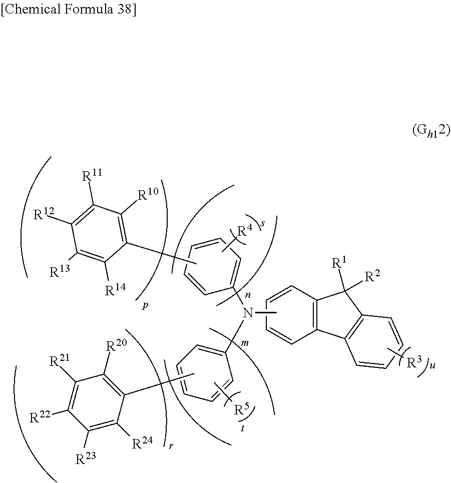

In General Formula (Gₕ₁2) above, each of m and r independently represents 1 or 2 and m+r is 2 or 3. Furthermore, t represents an integer of 0 to 4 and is preferably 0. R⁵ represents hydrogen or a hydrocarbon group having 1 to 3 carbon atoms. When m is 2, the kind and number of substituents and the position of bonds included in one phenylene group may be the same as or different from those of the other phenylene group; and when r is 2, the kind and number of substituents and the position of bonds included in one phenyl group may be the same as or different from those of the other phenyl group. In the case where t is an integer of 2 to 4, R⁵s may be the same as or different from each other, and adjacent groups of R⁵s may be bonded to each other to form a ring.

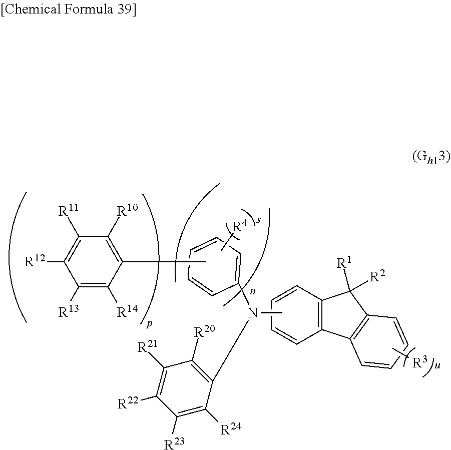

In General Formulae (Gₕ₁2) and (Gₕ₁3), each of n and p independently represents 1 or 2 and n+p is 2 or 3. In addition, s represents an integer of 0 to 4 and is preferably 0. R⁴ represents hydrogen or a hydrocarbon group having 1 to 3 carbon atoms. When n is 2, the kind and number of substituents and the position of bonds in one phenylene group may be the same as or different from those of the other phenylene group; and when p is 2, the kind and number of substituents and the position of bonds in one phenyl group may be the same as or different from those of the other phenyl group. In the case where s is an integer of 2 to 4, R⁴s may be the same as or different from each other.

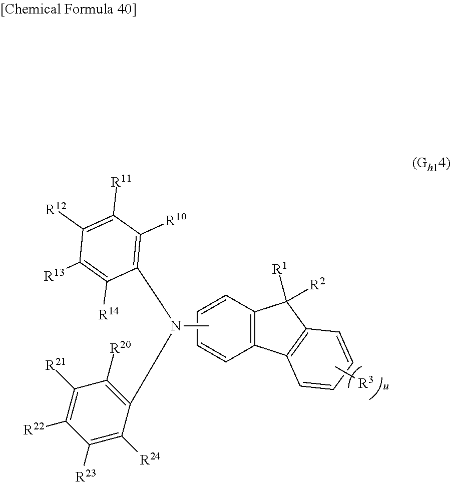

In General Formulae (Gₕ₁2) to (Gₕ₁4), each of R¹⁰ to R¹⁴ and R²⁰ to R²⁴ independently represents hydrogen or a hydrocarbon group having 1 to 12 carbon atoms forming a bond only by the sp3 hybrid orbitals. Note that at least three of R¹⁰ to R¹⁴ and at least three of R²⁰ to R²⁴ are preferably hydrogen. As the hydrocarbon group having 1 to 12 carbon atoms forming a bond only by the sp3 hybrid orbitals, a tert-butyl group and a cyclohexyl group are preferable. The total number of carbon atoms contained in R¹⁰ to R¹⁴ and R²⁰ to R²⁴ is 8 or more and the total number of carbon atoms contained in either R¹⁰ to R⁴ or R²⁰ to R²⁴ is 6 or more. Note that adjacent groups of R⁴, R¹⁰ to R⁴ and R²⁰ to R²⁴ may be bonded to each other to form a ring.

In General Formulae (Gₕ₁1) to (Gₕ₁4), u represents an integer of 0 to 4 and is preferably 0. Note that in the case where u is an integer of 2 to 4, R³s may be the same as or different from each other. In addition, each of R¹, R², and R³ independently represents an alkyl group having 1 to 4 carbon atoms, and R¹ and R² may be bonded to each other to form a ring.

An organic compound having an arylamine structure that has at least one aromatic group having first to third benzene rings and at least three alkyl groups is also preferable as the second organic compound having a hole-transport property that can be used in the hole-transport region 120. Note that the first to third benzene rings are bonded in this order, and the first benzene ring is directly bonded to nitrogen of amine.

The first benzene ring may further include a substituted or unsubstituted phenyl group and preferably includes an unsubstituted phenyl group. Furthermore, the second benzene ring or the third benzene ring may include a phenyl group substituted by an alkyl group.

Note that hydrogen is not directly bonded to carbon atoms at 1- and 3-positions in two or more of, preferably all of the first to third benzene rings, and the carbon atoms are bonded to any of the first to third benzene rings, the phenyl group substituted by the alkyl group, the at least three alkyl groups, and the nitrogen of the amine.

It is preferable that the organic compound further include a second aromatic group. The second aromatic group is preferably a group having an unsubstituted monocyclic ring or a substituted or unsubstituted bicyclic or tricyclic condensed ring, further preferably a group having a substituted or unsubstituted bicyclic or tricyclic condensed ring where the number of carbon atoms forming the ring is 6 to 13, still further preferably a group including a fluorene ring. Note that a dimethylfluorenyl group is preferable as the second aromatic group.

It is preferable that the organic compound further include a third aromatic group. The third aromatic group is a group having 1 to 3 substituted or unsubstituted benzene rings.

It is preferable that the at least three alkyl groups and the alkyl group substituted for the phenyl group be each a chain alkyl group having 2 to 5 carbon atoms. In particular, as the alkyl group, a chain alkyl group having a branch formed of 3 to 5 carbon atoms is preferable, and a t-butyl group is further preferable.

Examples of the above-described second organic compound having a hole-transport property include organic compounds having structures represented by (Gₕ₂1) to (Gₕ₂3) shown below.

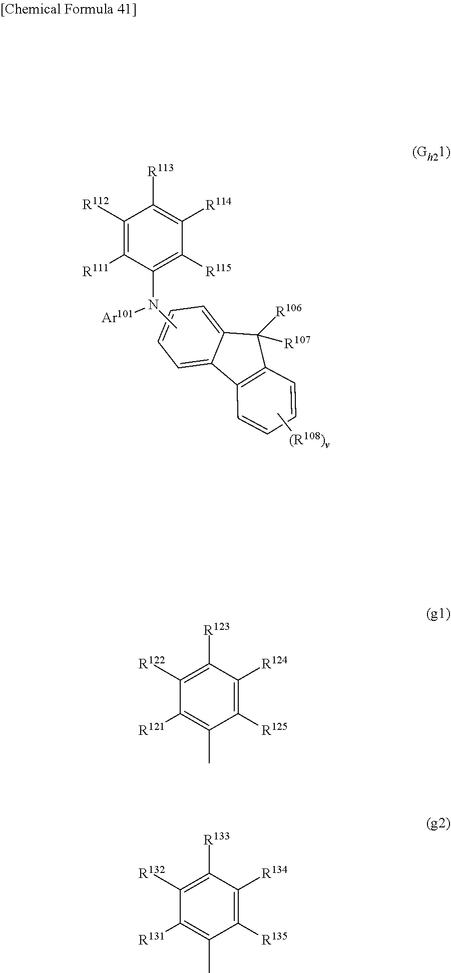

Note that in General Formula (Gₕ₂1), Ar¹⁰¹ represents a substituted or unsubstituted benzene ring or a substituent in which two or three substituted or unsubstituted benzene rings are bonded to one another.

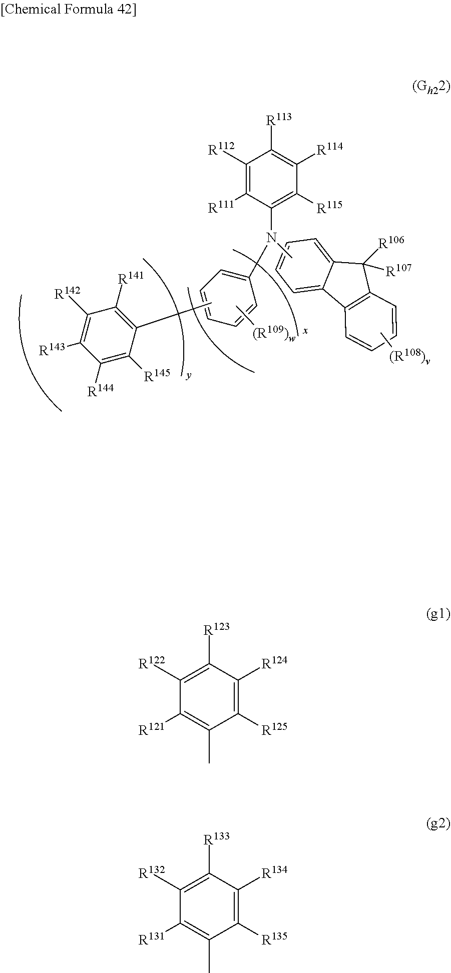

Note that in General Formula (Gₕ₂2) above, each of x and y independently represents 1 or 2 and x+y is 2 or 3. Furthermore, R¹⁰⁹ represents an alkyl group having 1 to 4 carbon atoms, and w represents an integer of 0 to 4. Each of R¹⁴¹ to R¹⁴⁵ independently represents any one of hydrogen, an alkyl group having 1 to 6 carbon atoms, and a cycloalkyl group having 5 to 12 carbon atoms. When w is 2 or more, R¹⁰⁹s may be the same as or different from each other. When x is 2, the kind and number of substituents and the position of bonds included in one phenylene group may be the same as or different from those of the other phenylene group. When y is 2, the kind and number of substituents included in one phenyl group including R¹⁴¹ to R¹⁴⁵ may be the same as or different from those of the other phenyl group including R¹⁴¹ to R¹⁴⁵.

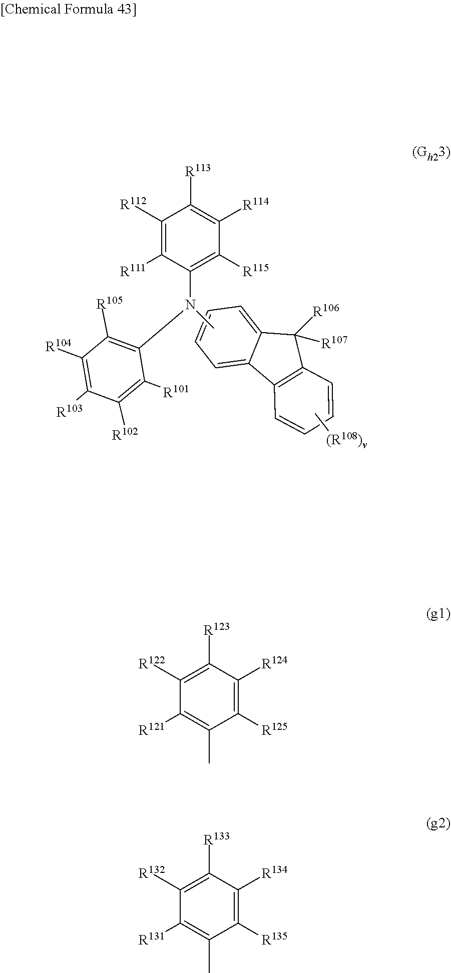

In General Formula (Gₕ₂3) above, each of R¹⁰¹ to R¹⁰⁵ independently represents any one of hydrogen, an alkyl group having 1 to 6 carbon atoms, a cycloalkyl group having 6 to 12 carbon atoms, and a substituted or unsubstituted phenyl group.

In General Formulae (G21) to (Gₕ₂3) above, each of R¹⁰⁶, R¹⁰⁷, and R¹⁰⁸ independently represents an alkyl group having 1 to 4 carbon atoms, and v represents an integer of 0 to 4. Note that when v is 2 or more, R¹⁰⁸s may be the same as or different from each other. One of R¹¹¹ to R¹¹⁵ represents a substituent represented by General Formula (g1) above, and the others each independently represent any one of hydrogen, an alkyl group having 1 to 6 carbon atoms, and a substituted or unsubstituted phenyl group. In General Formula (g1), one of R¹²¹ to R¹²⁵ represents a substituent represented by General Formula (g2) above, and the others each independently represent any one of hydrogen, an alkyl group having 1 to 6 carbon atoms, and a phenyl group substituted by an alkyl group having 1 to 6 carbon atoms. In General Formula (g2) above, each of R¹³¹ to R₁₃₅ independently represents any one of hydrogen, an alkyl group having 1 to 6 carbon atoms, and a phenyl group substituted by an alkyl group having 1 to 6 carbon atoms. Note that at least three of R₁₁₁ to R₁₁₅, R¹²¹ to R₁₂₅, and R₁₃₁ to R₁₃₅ are each an alkyl group having 1 to 6 carbon atoms; the number of substituted or unsubstituted phenyl groups in R¹¹¹ to R¹¹⁵ is one or less; and the number of phenyl groups substituted by an alkyl group having 1 to 6 carbon atoms in R¹²¹ to R¹²⁵ and R¹³¹ to R¹³⁵ is one or less. In at least two combinations of the three combinations R¹¹² and R¹¹⁴, R¹²² and R¹²⁴, and R¹³² and R¹³⁴, at least one R represents any of the substituents other than hydrogen.

The above-described second organic compound having a hole-transport property is an organic compound having a favorable hole-transport property and an ordinary refractive index higher than or equal to 1.50 and lower than or equal to 1.75 in the blue light emission region (greater than or equal to 455 nm and less than or equal to 465 nm) or an ordinary refractive index higher than or equal to 1.45 and lower than or equal to 1.70 with respect to light of 633 nm, which is typically used in refractive index measurement. A highly reliable organic compound having a high Tg at the same time can also be obtained. Such an organic compound having a hole-transport property has an enough hole-transport property and thus can be suitably used as a material of the hole-transport layer 112.

In the case of using the above-described second organic compound having a hole-transport property in the hole-injection layer 111, the organic compound having a hole-transport property mixed with a substance having an acceptor property is preferably used. Examples of the substance having an acceptor property include a compound having an electron-withdrawing group (a halogen group or a cyano group), such as 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyanonaphthoquinodimethane (abbreviation: F6-TCNNQ), or 2-(7-dicyanomethylene-1,3,4,5,6,8,9,10-octafluoro-7H-pyren-2-ylidene)malononitrile. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group or a halogen group such as a fluoro group) has a very high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the substance having an acceptor property, a molybdenum oxide, a vanadium oxide, a ruthenium oxide, a tungsten oxide, a manganese oxide, or the like can be used, other than the above-described organic compounds. Alternatively, the hole-injection layer 111 can be formed using a phthalocyanine-based complex compound such as phthalocyanine (abbreviation: H2Pc) or copper phthalocyanine (CuPc), an aromatic amine compound such as 4,4′-bis[N-(4-diphenylaminophenyl)-N-phenylamino]biphenyl (abbreviation: DPAB) or 4,4′-bis(N-(4-[N′-(3-methylphenyl)-N′-phenylamino]phenyl)-N-phenylamino)biphenyl (abbreviation: DNTPD), or a high molecule such as poly(3,4-ethylenedioxythiophene)/poly(styrenesulfonic acid) (PEDOT/PSS). The substance having an acceptor property can extract electrons from an adjacent hole-transport layer (or hole-transport material) by the application of an electric field.

In the case of forming the hole-injection layer 111 by mixing the above-described material having an acceptor property in the material having a hole-transport property, it is possible to select the material for forming the electrode regardless of the work function. In other words, besides a material having a high work function, a material having a low work function can also be used for the anode 101.

Next, examples of other structures and materials of the light-emitting device of one embodiment of the present invention are described. The light-emitting device of one embodiment of the present invention includes, as described above, the EL layer 103 formed of a plurality of layers between a pair of electrodes, the anode 101 and the cathode 102, and the EL layer 103 includes the light-emitting layer 113 containing a light-emitting material and the hole-transport region 120. Note that the hole-transport region 120 has a stacked-layer structure of the low refractive index layer and a layer containing a monoamine compound having the above structure.

The anode 101 is preferably formed using a metal, an alloy, or a conductive compound having a high work function (specifically, 4.0 eV or more), a mixture thereof, or the like. Specifically, for example, indium oxide-tin oxide (ITO: Indium Tin Oxide), indium oxide-tin oxide containing silicon or silicon oxide, indium oxide-zinc oxide, indium oxide containing tungsten oxide and zinc oxide (IWZO), and the like can be given. These conductive metal oxide films are usually formed by a sputtering method but may also be formed by application of a sol-gel method or the like. An example of the formation method is a method in which indium oxide-zinc oxide is formed by a sputtering method using a target in which 1 to 20 wt % zinc oxide is added to indium oxide. Indium oxide containing tungsten oxide and zinc oxide (IWZO) can also be formed by a sputtering method using a target containing 0.5 to 5 wt % tungsten oxide and 0.1 to 1 wt % zinc oxide with respect to indium oxide. In addition, gold (Au), platinum (Pt), nickel (Ni), tungsten (W), chromium (Cr), molybdenum (Mo), iron (Fe), cobalt (Co), copper (Cu), palladium (Pd), a nitride of a metal material (such as titanium nitride), and the like can be given as examples of the material that is used for the anode 101. Graphene can also be used for the material that is used for the anode 101. Note that when a composite material described later is used for a layer that is in contact with the anode 101 in the EL layer 103, an electrode material can be selected regardless of its work function.

When the anode 101 is formed using a material having a transmitting property with respect to visible light, the light-emitting device can emit light from the cathode side as illustrated in FIG. 1C. When the anode 101 is formed on the substrate side, the light-emitting device can be what is called a bottom-emission light-emitting device.

Although the EL layer 103 preferably has a stacked-layer structure, there is no particular limitation on the stacked-layer structure, and various functional layers such as a hole-injection layer, a hole-transport layer, a light-emitting layer, an electron-transport layer, an electron-injection layer, a carrier-blocking layer (a hole-blocking layer, an electron-blocking layer), an exciton-blocking layer, an intermediate layer, and a charge-generation layer can be employed. Note that one or more of the above layers are not necessarily provided. In this embodiment, two kinds of structures are described: the structure including the hole-injection layer 111, the hole-transport layer 112, the electron-transport layer 114, and the electron-injection layer 115 in addition to the light-emitting layer 113 as illustrated in FIG. 1A; and the structure including a charge-generation layer 116 in addition to the electron-transport layer 114, the light-emitting layer 113, the hole-injection layer 111, and the hole-transport layer 112 as illustrated in FIG. 1B. Hereinafter, materials that can form the functional layers in the case where each of the functional layers is not a low refractive index layer are specifically described.

The hole-injection layer 111 is a layer containing a substance having an acceptor property. Either an organic compound or an inorganic compound can be used as the substance having an acceptor property.

As the substance having an acceptor property, a compound having an electron-withdrawing group (a halogen group or a cyano group) can be used; 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyanonaphthoquinodimethane (abbreviation: F6-TCNNQ), 2-(7-dicyanomethylene-1,3,4,5,6,8,9,10-octafluoro-7H-pyren-2-ylidene)malononitrile, and the like can be given. In particular, a compound in which electron-withdrawing groups are bonded to a condensed aromatic ring having a plurality of heteroatoms, such as HAT-CN, is preferable because it is thermally stable. A [3]radialene derivative having an electron-withdrawing group (in particular, a halogen group such as a fluoro group, or a cyano group) has a very high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a molybdenum oxide, a vanadium oxide, a ruthenium oxide, a tungsten oxide, a manganese oxide, or the like can be used, other than the above-described organic compounds. Alternatively, the hole-injection layer 111 can be formed using a phthalocyanine-based complex compound such as phthalocyanine (abbreviation: H₂Pc) or copper phthalocyanine (CuPc), an aromatic amine compound such as 4,4′-bis[N-(4-diphenylaminophenyl)-N-phenylamino]biphenyl (abbreviation: DPAB) or N,N′-bis{4-[bis(3-methylphenyl)amino]phenyl}-N,N′-diphenyl-(1,1′-biphenyl)-4,4′-diamine (abbreviation: DNTPD), or a high molecule such as poly(3,4-ethylenedioxythiophene)/poly(styrenesulfonic acid) (PEDOT/PSS). The substance having an acceptor property can extract electrons from an adjacent hole-transport layer (or hole-transport material) by the application of an electric field.

Alternatively, a composite material in which a material having a hole-transport property contains the above-described acceptor substance can be used for the hole-injection layer 111. By using a composite material in which a material having a hole-transport property contains an acceptor substance, a material used to form an electrode can be selected regardless of its work function. In other words, besides a material having a high work function, a material having a low work function can also be used for the anode 101.

As the material having a hole-transport property used for the composite material, any of a variety of organic compounds such as aromatic amine compounds, carbazole derivatives, aromatic hydrocarbons, and high molecular compounds (e.g., oligomers, dendrimers, or polymers) can be used. Note that the material having a hole-transport property used for the composite material is preferably a substance having a hole mobility higher than or equal to 1×10⁻⁶ cm²/Vs. Organic compounds which can be used as the material having a hole-transport property in the composite material are specifically given below.

Examples of the aromatic amine compounds that can be used for the composite material include N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis{4-[bis(3-methylphenyl)amino]phenyl}-N,N′-diphenyl-(1,1′-biphenyl)-4,4′-diamine (abbreviation: DNTPD), and 1,3,5-tris[N-(4-diphenylaminophenyl)-N-phenylamino]benzene (abbreviation: DPA3B). Specific examples of the carbazole derivatives include 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(N-carbazolyl)]phenyl-10-phenylanthracene (abbreviation: CzPA), and 1,4-bis[4-(N-carbazolyl)phenyl]-2,3,5,6-tetraphenylbenzene. Examples of the aromatic hydrocarbon include 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra(tert-butyl)perylene. Other examples include pentacene and coronene. The aromatic hydrocarbon may have a vinyl skeleton. Examples of the aromatic hydrocarbon having a vinyl skeleton include 4,4′-bis(2,2-diphenylvinyl)biphenyl (abbreviation: DPVBi) and 9,10-bis[4-(2,2-diphenylvinyl)phenyl]anthracene (abbreviation: DPVPA).

Other examples include high molecular compounds such as poly(N-vinylcarbazole) (abbreviation: PVK), poly(4-vinyltriphenylamine) (abbreviation: PVTPA), poly[N-(4-(N′-[4-(4-diphenylamino)phenyl]phenyl-N′-phenylamino)phenyl)methacrylamide] (abbreviation: PTPDMA), and poly[N,N′-bis(4-butylphenyl)-N,N′-bis(phenyl)benzidine] (abbreviation: Poly-TPD).

The material having a hole-transport property used in the composite material further preferably has any of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that includes a naphthalene ring, or an aromatic monoamine in which a 9-fluorenyl group is bonded to nitrogen of amine through an arylene group may be used. Note that the organic compound preferably has an N,N-bis(4-biphenyl)amino group because a light-emitting device having a favorable lifetime can be fabricated. Specific examples of the organic compound include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi(9H-fluoren)-2-amine (abbreviation: PCBNBSF), N,N-bis([1,1′-biphenyl]-4-yl)-9,9′-spirobi[9H-fluoren]-2-amine (abbreviation: BBASF), N,N-bis([1,1′-biphenyl]-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi(9H-fluoren)-4-amine (abbreviation: oFBiSF), N-(4-biphenyl)-N-(9,9-dimethyl-9H-fluoren-2-yl)dibenzofuran-4-amine(abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(1,1′-biphenyl-4-yl)-9,9-dimethyl-N-[4-(9-phenyl-9H-carbazol-3-yl)phenyl]-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Further preferably, the material having a hole-transport property that is used in the composite material is a substance having a relatively deep HOMO level higher than or equal to −5.7 eV and lower than or equal to −5.4 eV. When the material having a hole-transport property that is used in the composite material has a relatively deep HOMO level, holes can be easily injected into the hole-transport layer 112 to easily provide a light-emitting device having a favorable lifetime. In addition, when the material having a hole-transport property that is used in the composite material is a substance having a relatively deep HOMO level, induction of holes can be inhibited properly so that the light-emitting device can have a more favorable lifetime.

Note that mixing the above composite material with a fluoride of an alkali metal or an alkaline earth metal (the proportion of fluorine atoms in the layer is preferably higher than or equal to 20%) can lower the refractive index of the layer. This also enables a layer with a low refractive index to be formed in the EL layer 103, leading to higher external quantum efficiency of the light-emitting device.

The formation of the hole-injection layer 111 can improve the hole-injection property, offering the light-emitting device with a low driving voltage.

Among substances having an acceptor property, the organic compound having an acceptor property is easy to use because it is easily deposited by vapor deposition.

The hole-transport layer 112 is formed containing a material having a hole-transport property. The material having a hole-transport property preferably has a hole mobility higher than or equal to 1×10⁻⁶ cm²/Vs.

Examples of the material having a hole-transport property include a compound having an aromatic amine skeleton, such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), or N-phenyl-N-[4-(9-phenyl-9H-carbazol-3-yl)phenyl]-9,9′-spirobi[9H-fluoren]-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 3,3′-bis(9-phenyl-9H-carbazole) (abbreviation: PCCP); a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV); and a compound having a furan skeleton, such as 4,4′,4″-(benzene-1,3,5-triyl)tri(dibenzofuran) (abbreviation: DBF3P-II) or 4-(3-[3-(9-phenyl-9H-fluoren-9-yl)phenyl]phenyl)dibenzofuran (abbreviation: mmDBFFLBi-II). Among the above materials, the compound having an aromatic amine skeleton and the compound having a carbazole skeleton are preferable because these have favorable reliability and have high hole-transport properties to contribute to a reduction in driving voltage. Note that any of the substances given as examples of the material having a hole-transport property that is used for the composite material for the hole-injection layer 111 can also be suitably used as the material included in the hole-transport layer 112.

The light-emitting layer 113 includes a light-emitting substance and a host material. The light-emitting layer 113 may additionally include other materials. Alternatively, the light-emitting layer 113 may be a stack of two layers with different compositions.

As the light-emitting substance, fluorescent substances, phosphorescent substances, substances exhibiting thermally activated delayed fluorescence (TADF), or other light-emitting substances may be used. Note that one embodiment of the present invention can more suitably be used in the case where the light-emitting layer 113 is a layer that exhibits fluorescence, specifically, blue fluorescence.

Examples of the material that can be used as a fluorescent substance in the light-emitting layer 113 are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone, (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl)-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine] (abbreviation:1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, or high reliability.

Examples of the material that can be used when a phosphorescent substance is used as the light-emitting substance in the light-emitting layer 113 are as follows.

The examples include an organometallic iridium complex having a 4H-triazole skeleton, such as tris{2-[5-(2-methylphenyl)-4-(2,6-dimethylphenyl)-4H-1,2,4-triazol-3-yl-κN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), or tris[4-(3-biphenyl)-5-isopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) or tris(1-methyl-5-phenyl-3-propyl-1H-1,2,4-triazolato)iridium(III) (abbreviation: [Ir(Prptz1-Me)₃]); an organometallic iridium complex having an imidazole skeleton, such as fac-tris[(1-2,6-diisopropylphenyl)-2-phenyl-1H-imidazole]iridium(III) (abbreviation: [Ir(iPrpmi)₃]) or tris[3-(2,6-dimethylphenyl)-7-methylimidazo[1,2-f]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), or bis[2-(4′,6′-difluorophenyl)pyridinato-N,C^{2′}]iridium(III) acetylacetonate (abbreviation: FIracac). These compounds exhibit blue phosphorescence and have an emission peak in the wavelength range from 440 nm to 520 nm.

Other examples include an organometallic iridium complex having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), or (acetylacetonato)bis(4,6-diphenylpyrimidinato)iridium(III) (abbreviation: [Ir(dppm)₂(acac)]); an organometallic iridium complex having a pyrazine skeleton, such as (acetylacetonato)bis(3,5-dimethyl-2-phenylpyrazinato)iridium(III) (abbreviation: [Ir(mppr-Me)₂(acac)]) or (acetylacetonato)bis(5-isopropyl-3-methyl-2-phenylpyrazinato)iridium(III) (abbreviation: [Ir(mppr-iPr)₂(acac)]); an organometallic iridium complex having a pyridine skeleton, such as tris(2-phenylpyridinato-N,C^{2′})iridium(III) (abbreviation: [Ir(ppy)₃]), bis(2-phenylpyridinato-N,C^{2′})iridium(II) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), or bis(2-phenylquinolinato-N,C^{2′})iridium(III) acetylacetonate (abbreviation: [Ir(pq)₂(acac)]); and a rare earth metal complex such as tris(acetylacetonatoxmonophenanthroline)terbium(III) (abbreviation: [Tb(acac)₃(Phen)]). These are mainly compounds that exhibit green phosphorescence and have an emission peak in the wavelength range from 500 nm to 600 nm. Note that organometallic iridium complexes having a pyrimidine skeleton have distinctively high reliability or emission efficiency and thus are particularly preferable.

Other examples include an organometallic iridium complex having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), or bis[4,6-di(naphthalen-1-yl)pyrimidinato](dipivaloylmethanato)iridium(III) (abbreviation: [Ir(d1npm)₂(dpm)]); an organometallic iridium complex having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinatoxdipivaloylmethanato)iridium(III) (abbreviation: [Ir(tppr)₂(dpm)]), or (acetylacetonato)bis[2,3-bis(4-fluorophenyl)quinoxalinato]iridium(III) (abbreviation: [Ir(Fdpq)₂(acac)]); an organometallic iridium complex having a pyridine skeleton, such as tris(1-phenylisoquinolinato-N,C^{2′})iridium(III) (abbreviation: [Ir(piq)₃]) or bis(1-phenylisoquinolinato-N,C^{2′})iridium(III) acetylacetonate (abbreviation: [Ir(piq)₂(acac)]); a platinum complex such as 2,3,7,8,12,13,17,18-octaethyl-21H,23H-porphyrinplatinum(II) (abbreviation: PtOEP); and a rare earth metal complex such as tris(1,3-diphenyl-1,3-propanedionatoxmonophenanthroline)europium(III) (abbreviation: [Eu(DBM)₃(Phen)]) or tris[1-(2-thenoyl)-3,3,3-trifluoroacetonato](monophenanthroline)europium(III) (abbreviation: [Eu(TTA)₃(Phen)]). These compounds exhibit red phosphorescence and have an emission peak in the wavelength range from 600 nm to 700 nm. Organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

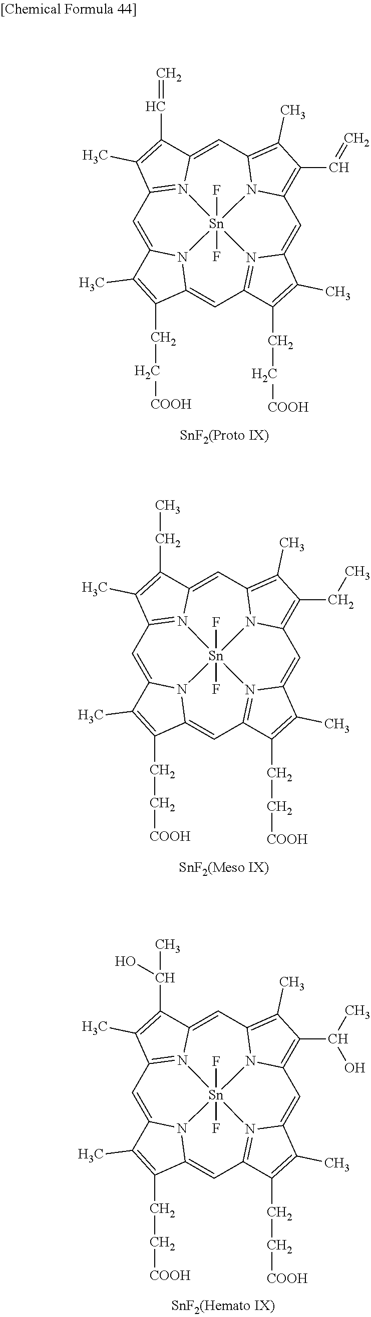

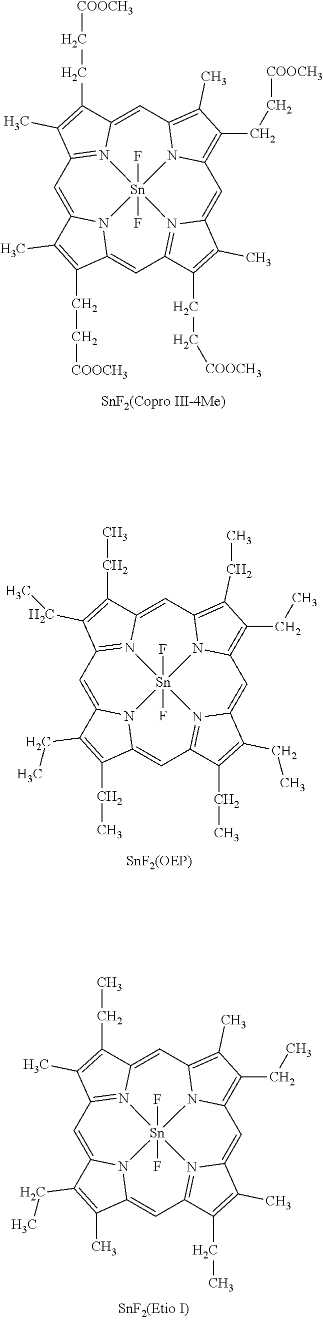

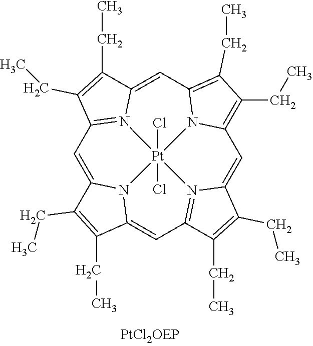

Alternatively, aheterocyclic compound having one or both of a π-electron rich heteroaromatic ring and a π-electron deficient heteroaromatic ring that is represented by the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 9-[4-(4,6-diphenyl-1,3,5-triazin-2-yl)phenyl]-9′-phenyl-9H,9′H-3,3′-bicarbazol (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA) can be used. Such a heterocyclic compound is preferable because of having excellent electron-transport and hole-transport properties owing to a π-electron rich heteroaromatic ring and a π-electron deficient heteroaromatic ring. Among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferred because of their stability and favorable reliability. In particular, a benzofuropyrimidine skeleton, a benzothienopyrimidine skeleton, a benzofuropyrazine skeleton, and a benzothienopyrazine skeleton are preferred because of their high acceptor properties and favorable reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have stability and favorable reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which the π-electron rich heteroaromatic ring is directly bonded to the π-electron deficient heteroaromatic ring is particularly preferred because the electron-donating property of the π-electron rich heteroaromatic ring and the electron-accepting property of the π-electron deficient heteroaromatic ring are both improved, the energy difference between the Si level and the T1 level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a cyano group or a nitrile group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

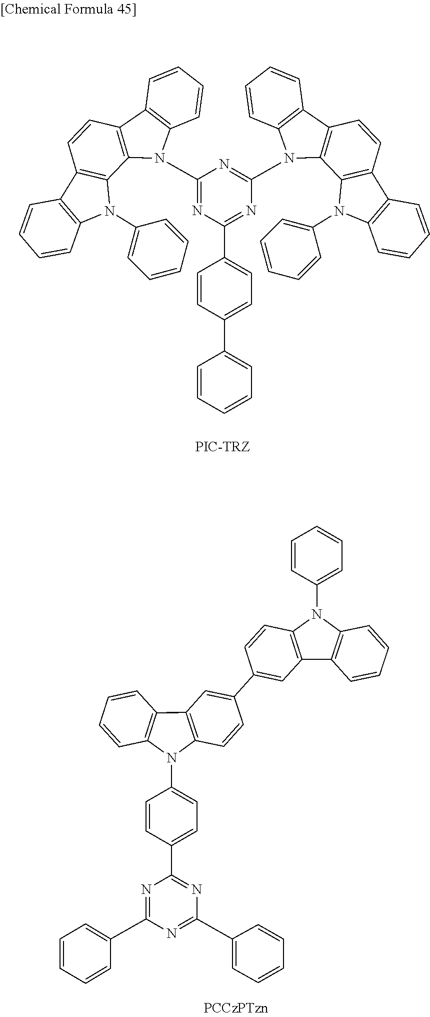 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

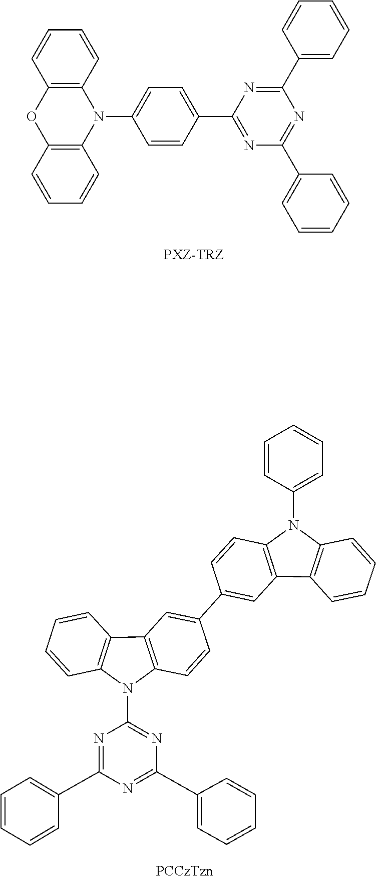 [SMILES: c1ccc(-c2nc(-c3ccccc3)nc(-c3ccc(N4c5ccccc5Oc5ccccc54)cc3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

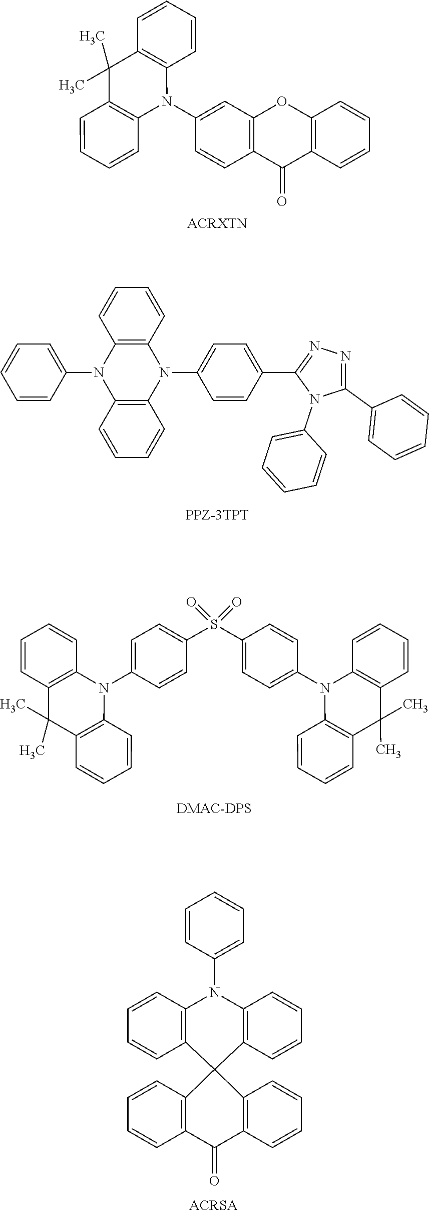

Note that a TADF material is a material having a small difference between the Si level and the T1 level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light.

An exciplex whose excited state is formed of two kinds of substances has an extremely small difference between the Si level and the T1 level and functions as a TADF material capable of converting triplet excitation energy into singlet excitation energy.

A phosphorescent spectrum observed at a low temperature (e.g., 77 K to 10 K) is used for an index of the T1 level. When the level of energy with a wavelength of the line obtained by extrapolating a tangent to the fluorescent spectrum at a tail on the short wavelength side is the Si level and the level of energy with a wavelength of the line obtained by extrapolating a tangent to the phosphorescent spectrum at a tail on the short wavelength side is the T1 level, the difference between S1 and T1 of the TADF material is preferably smaller than or equal to 0.3 eV, further preferably smaller than or equal to 0.2 eV.

When a TADF material is used as the light-emitting substance, the Si level of the host material is preferably higher than the Si level of the TADF material. In addition, the T1 level of the host material is preferably higher than the T1 level of the TADF material.

As the host material in the light-emitting layer, various carrier-transport materials such as materials having an electron-transport property, materials having a hole-transport property, and the above-described TADF materials can be used.

The material having a hole-transport property is preferably an organic compound having an amine skeleton or a π-electron rich heteroaromatic ring skeleton. Examples of the material include a compound having an aromatic amine skeleton, such as 4,4′-bis[N-(1-naphthyl)-N-phenylamino]biphenyl (abbreviation: NPB), N,N-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), or N-phenyl-N-[4-(9-phenyl-9H-carbazol-3-yl)phenyl]-9,9′-spirobi[9H-fluoren]-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 3,3′-bis(9-phenyl-9H-carbazole) (abbreviation: PCCP); a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV); and a compound having a furan skeleton, such as 4,4′,4″-(benzene-1,3,5-triyl)tri(dibenzofuran) (abbreviation: DBF3P-II) or 4-(3-[3-(9-phenyl-9H-fluoren-9-yl)phenyl]phenyl)dibenzofuran (abbreviation: mmDBFFLBi-II). Among the above materials, the compound having an aromatic amine skeleton and the compound having a carbazole skeleton are preferable because these have favorable reliability and have high hole-transport properties to contribute to a reduction in driving voltage. In addition, the organic compounds given as examples of the material having a hole-transport property that can be used for the hole-transport layer 112 can also be used.

As the material having an electron-transport property, for example, a metal complex such as bis(10-hydroxybenzo[h]quinolinato)beryllium(II) (abbreviation: BeBqz), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ); or an organic compound having a π-electron deficient heteroaromatic ring is preferable. Examples of the organic compound having a π-electron deficient heteroaromatic ring skeleton include a heterocyclic compound having a polyazole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), or 2-[3-(dibenzothiophen-4-yl)phenyl]-1-phenyl-1H-benzimidazole (abbreviation: mDBTBIm-II); a heterocyclic compound having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline(abbreviation: 2mCzBPDBq), 4,6-bis[3-(phenanthren-9-yl)phenyl]pyrimidine (abbreviation: 4,6mPnP2Pm), or 4,6-bis[3-(4-dibenzothienyl)phenyl]pyrimidine (abbreviation: 4,6mDBTP2Pm-II); a heterocyclic compound having a pyridine skeleton, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB); and a heterocyclic compound having a triazine skeleton, such as 2-[3′-(9,9-dimethyl-9H-fluoren-2-yl)-1,1′-biphenyl-3-yl]-4,6-diphenyl-1,3,5-triazine (abbreviation: mFBPTzn), 2-[(1,1′-biphenyl)-4-yl]-4-phenyl-6-[9,9′-spirobi(9H-fluoren)-2-yl]-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), or 2-{3-[3-(benzo[b]naphtho[1,2-d]furan-6-yl)phenyl]phenyl}-4,6-diphenyl-1,3,5-triazine (abbreviation: mBnfBPTzn-02). Among the above materials, the heterocyclic compound having a diazine skeleton, the heterocyclic compound having a pyridine skeleton, and the heterocyclic compound having a triazine skeleton have favorable reliability and thus are preferable. In particular, the heterocyclic compound having a diazine (pyrimidine or pyrazine) skeleton and the heterocyclic compound having a triazine skeleton have a high electron-transport property to contribute to a reduction in driving voltage.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S1 level of the TADF material is preferably higher than the Si level of the fluorescent substance in order that high emission efficiency can be achieved. Furthermore, the T1 level of the TADF material is preferably higher than the Si level of the fluorescent substance. Therefore, the T1 level of the TADF material is preferably higher than the T1 level of the fluorescent substance.

It is also preferable to use a TADF material that exhibits light emission overlapping with the wavelength of a lowest-energy-side absorption band of the fluorescent substance, in which case excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be distanced from each other with little influence on carrier transportation or carrier recombination. Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroaromatic ring. Examples of the condensed aromatic ring or the condensed heteroaromatic ring include a phenanthrene skeleton, a stilbene skeleton, an acridone skeleton, a phenoxazine skeleton, and a phenothiazine skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferred because of its high fluorescence quantum yield.

In the case where a fluorescent substance is used as the light-emitting substance, a material having an anthracene skeleton is suitably used as the host material. The use of a substance having an anthracene skeleton as the host material for the fluorescent substance makes it possible to obtain a light-emitting layer with favorable emission efficiency and favorable durability. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further condensed to carbazole because the HOMO level thereof is shallower than that of carbazole by approximately 0.1 eV and thus holes enter the host material easily. In particular, the host material preferably has a dibenzocarbazole skeleton because the HOMO level thereof is shallower than that of carbazole by approximately 0.1 eV so that holes enter the host material easily, the hole-transport property is improved, and the heat resistance is increased. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(N-carbazolyl)phenyl-10-phenylanthracene (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-(4-(9-phenyl-9H-fluoren-9-yl)biphenyl-4′-yl)anthracene (abbreviation: FLPPA), and 9-(1-naphthyl)-10-[4-(2-naphthyl)phenyl]anthracene (abbreviation: αN-βNPAnth). In particular, CzPA, cgDBCzPA, 2mBnfPPA, and PCzPA exhibit extremely favorable properties and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of these mixed materials. These mixed materials are preferably selected so as to form an exciplex that exhibits light emission whose wavelength overlaps with the wavelength of a lowest-energy-side absorption band of the light-emitting substance, in which case energy can be transferred smoothly and light emission can be obtained efficiently. The use of such a structure is preferable because the driving voltage can also be reduced.

Note that at least one of the materials forming an exciplex may be a phosphorescent substance. In this case, triplet excitation energy can be efficiently converted into singlet excitation energy by reverse intersystem crossing.

Combination of a material having an electron-transport property and a material having a hole-transport property whose HOMO level is higher than or equal to the HOMO level of the material having an electron-transport property is preferable for forming an exciplex efficiently. In addition, the LUMO level of the material having a hole-transport property is preferably higher than or equal to the LUMO level of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength side than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient PL lifetime of the mixed film has longer lifetime components or has a larger proportion of delayed components than that of each of the materials, observed by comparison of transient photoluminescence (PL) of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

The electron-transport layer 114 is a layer containing a substance having an electron-transport property. As the substance having an electron-transport property, it is possible to use the above-described substance having an electron-transport property that can be used as the host material.

The electron mobility of the electron-transport layer 114 of the case where the square root of the electric field strength [V/cm] is 600 is preferably higher than or equal to 1×10⁻⁷ cm²/Vs and lower than or equal to 5×10⁻³ cm²/Vs. The amount of electrons injected into the light-emitting layer can be controlled by the reduction in the electron-transport property of the electron-transport layer 114, whereby the light-emitting layer can be prevented from having excess electrons. It is particularly preferable to employ this structure when the hole-injection layer is formed using a composite material that includes a material having a hole-transport property with a relatively deep HOMO level of −5.7 eV or higher and −5.4 eV or lower, in which case a favorable lifetime can be achieved. In this case, the material having an electron-transport property preferably has a HOMO level of −6.0 eV or higher.

There is preferably a difference in the concentration (including 0) of the alkali metal or the metal complex of the alkali metal in the electron-transport layer 114 in the thickness direction.

A layer including an alkali metal, an alkaline earth metal, a compound thereof, or a complex thereof such as lithium fluoride (LiF), cesium fluoride (CsF), calcium fluoride (CaF₂), or 8-quinolinolato-lithium (abbreviation: Liq) may be provided as the electron-injection layer 115 between the electron-transport layer 114 and the cathode 102. For example, an electride or a layer that is formed using a substance having an electron-transport property and that includes an alkali metal, an alkaline earth metal, or a compound thereof can be used as the electron-injection layer 115. Examples of the electride include a substance in which electrons are added at high concentration to a mixed oxide of calcium and aluminum.

Note that as the electron-injection layer 115, it is possible to use a layer including a substance that has an electron-transport property (preferably an organic compound having a bipyridine skeleton) and includes a fluoride of the alkali metal or the alkaline earth metal at a concentration higher than or equal to that at which the electron-injection layer 115 becomes in a microcrystalline state (50 wt % or higher). Since the layer has a low refractive index, a light-emitting device including the layer can have favorable external quantum efficiency.

Instead of the electron-injection layer 115 in FIG. 1A, the charge-generation layer 116 may be provided (FIG. 1B). The charge-generation layer 116 refers to a layer capable of injecting holes into a layer in contact with the cathode side of the charge-generation layer and electrons into a layer in contact with the anode side thereof when a potential is applied. The charge-generation layer 116 includes at least a p-type layer 117. The p-type layer 117 is preferably formed using any of the composite materials given above as examples of materials that can be used for the hole-injection layer 111. The p-type layer 117 may be formed by stacking a film including the above-described acceptor material as a material included in the composite material and a film including a hole-transport material. When a potential is applied to the p-type layer 117, electrons are injected into the electron-transport layer 114 and holes are injected into the cathode 102; thus, the light-emitting device operates

Note that the charge-generation layer 116 preferably includes one or both of an electron-relay layer 118 and an electron-injection buffer layer 119 in addition to the p-type layer 117.

The electron-relay layer 118 includes at least the substance having an electron-transport property and has a function of preventing an interaction between the electron-injection buffer layer 119 and the p-type layer 117 and smoothly transferring electrons. The LUMO level of the substance having an electron-transport property included in the electron-relay layer 118 is preferably between the LUMO level of the acceptor substance in the p-type layer 117 and the LUMO level of a substance included in a layer of the electron-transport layer 114 that is in contact with the charge-generation layer 116. As a specific value of the energy level, the LUMO level of the substance having an electron-transport property in the electron-relay layer 118 is preferably higher than or equal to −5.0 eV, further preferably higher than or equal to −5.0 eV and lower than or equal to −3.0 eV. Note that as the substance having an electron-transport property in the electron-relay layer 118, a phthalocyanine-based material or a metal complex having a metal-oxygen bond and an aromatic ligand is preferably used.

The electron-injection buffer layer 119 can be formed using a substance having a high electron-injection property, e.g., an alkali metal, an alkaline earth metal, a rare earth metal, or a compound thereof (an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)).

In the case where the electron-injection buffer layer 119 contains the substance having an electron-transport property and a donor substance, an organic compound such as tetrathianaphthacene (abbreviation: TTN), nickelocene, or decamethylnickelocene can be used as the donor substance, as well as an alkali metal, an alkaline earth metal, a rare earth metal, or a compound thereof (e.g., an alkali metal compound (including an oxide such as lithium oxide, a halide, and a carbonate such as lithium carbonate and cesium carbonate), an alkaline earth metal compound (including an oxide, a halide, and a carbonate), or a rare earth metal compound (including an oxide, a halide, and a carbonate)).

As the substance having an electron-transport property, a material similar to the above-described material for the electron-transport layer 114 can be used. Since the above-described material is an organic compound having a low refractive index, the use of the material for the electron-injection buffer layer 119 can offer a light-emitting device with favorable external quantum efficiency.

As a substance of the cathode 102, any of metals, alloys, and electrically conductive compounds with a low work function (specifically, lower than or equal to 3.8 eV), mixtures thereof, and the like can be used. Specific examples of such a cathode material include elements belonging to Group 1 and Group 2 of the periodic table, such as alkali metals (e.g., lithium (Li) and cesium (Cs)), magnesium (Mg), calcium (Ca), and strontium (Sr), alloys containing these elements (e.g., MgAg and AlLi), rare earth metals such as europium (Eu) and ytterbium (Yb), and alloys containing these rare earth metals. However, when the electron-injection layer is provided between the cathode 102 and the electron-transport layer, any of a variety of conductive materials such as Al, Ag, ITO, or indium oxide-tin oxide containing silicon or silicon oxide can be used for the cathode 102 regardless of the work function.

In the case where the cathode 102 is formed using a material that transmits visible light, the light-emitting device can emit light from the cathode side as illustrated in FIG. 1D. In the case where the anode 101 is formed on the substrate side, the light-emitting device can be what is called a top-emission light-emitting device.

Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

Any of a variety of methods can be used for forming the EL layer 103, regardless of a dry method or a wet method. For example, a vacuum evaporation method, a gravure printing method, an offset printing method, a screen printing method, an ink-jet method, a spin coating method, or the like may be used.

Different methods may be used to form the electrodes or the layers described above.

The structure of the layers provided between the anode 101 and the cathode 102 is not limited to the above-described structure. Preferably, a light-emitting region where holes and electrons recombine is positioned away from the anode 101 and the cathode 102 so as to inhibit quenching due to the proximity of the light-emitting region and a metal used for electrodes or carrier-injection layers.

Furthermore, in order that transfer of energy from an exciton generated in the light-emitting layer can be inhibited, preferably, the hole-transport layer or the electron-transport layer, which is in contact with the light-emitting layer 113, particularly a carrier-transport layer closer to the recombination region in the light-emitting layer 113, is preferably formed using a substance having a wider band gap than the light-emitting material of the light-emitting layer or the light-emitting material included in the light-emitting layer.

Next, an embodiment of a light-emitting device with a structure in which a plurality of light-emitting units are stacked (also referred to as a stacked device or a tandem device) is described. This light-emitting device includes a plurality of light-emitting units between an anode and a cathode. One light-emitting unit has substantially the same structure as the EL layer 103 illustrated in FIG. 1A. In other words, the light-emitting device illustrated in FIG. 1A or FIG. 1B includes a single light-emitting unit, and the tandem device includes a plurality of light-emitting units.

In the tandem device, a first light-emitting unit and a second light-emitting unit are stacked between an anode and a cathode, and a charge-generation layer is provided between the first light-emitting unit and the second light-emitting unit. The anode and the cathode correspond, respectively, to the anode 101 and the cathode 102 in FIG. 1A, and the same materials as those given in the description for FIG. 1A can be used. The first light-emitting unit and the second light-emitting unit may have the same structure or different structures.

The charge-generation layer in the tandem device has a function of injecting electrons into one of the light-emitting units and injecting holes into the other of the light-emitting units when voltage is applied between the anode and the cathode. That is, the charge-generation layer injects electrons into the first light-emitting unit 1 and holes into the second light-emitting unit when voltage is applied such that the potential of the anode becomes higher than the potential of the cathode.

The charge-generation layer preferably has a structure similar to that of the charge-generation layer 116 described with reference to FIG. 1B. A composite material of an organic compound and a metal oxide has an excellent carrier-injection property and an excellent carrier-transport property; thus, low-voltage driving and low-current driving can be achieved. In the case where the anode-side surface of a light-emitting unit is in contact with the charge-generation layer, the charge-generation layer can also function as a hole-injection layer of the light-emitting unit; therefore, a hole-injection layer is not necessarily provided in the light-emitting unit.

In the case where the charge-generation layer of the tandem device includes the electron-injection buffer layer 119, the electron-injection buffer layer 119 functions as the electron-injection layer in the light-emitting unit on the anode side; thus, an electron-injection layer is not necessarily formed in the light-emitting unit on the anode side.

The tandem device having two light-emitting units is described above; one embodiment of the present invention can also be applied to a tandem device in which three or more light-emitting units are stacked. With a plurality of light-emitting units partitioned by the charge-generation layer between a pair of electrodes, it is possible to provide a long-life device that can emit light with high luminance at a low current density. A light-emitting apparatus that can be driven at a low voltage and has low power consumption can be provided.

When the emission colors of the light-emitting units are different, light emission of a desired color can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as a whole.

The above-described layers or electrodes such as the EL layer 103, the first light-emitting unit, the second light-emitting unit, and the charge-generation layer can be formed by a method such as an evaporation method (including a vacuum evaporation method), a droplet discharge method (also referred to as an ink-jet method), a coating method, or a gravure printing method, for example. A low molecular material, a middle molecular material (including an oligomer and a dendrimer), or a high molecular material may be included in the layers or electrodes.

This embodiment can be freely combined with any of the other embodiments.

### Embodiment 2

In this embodiment, a light-emitting apparatus using the light-emitting device described in Embodiment 1 will be described.

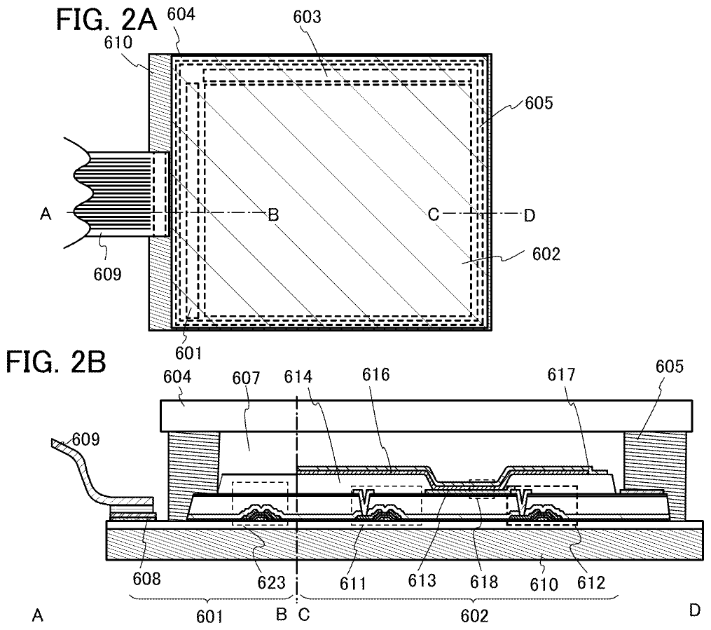

In this embodiment, a light-emitting apparatus manufactured using the light-emitting device described in Embodiment 1 is described with reference to FIG. 2A and FIG. 2B. Note that FIG. 2A is a top view of the light-emitting apparatus and FIG. 2B is a cross-sectional view taken along the dashed-dotted line A-B and the dashed-dotted line C-D in FIG. 2A. This light-emitting apparatus includes a driver circuit portion (source line driver circuit) 601, a pixel portion 602, and a driver circuit portion (gate line driver circuit) 603, which are to control light emission of a light-emitting device and illustrated with dotted lines. Reference numeral 604 denotes a sealing substrate; 605, a sealing material; and 607, a space surrounded by the sealing material 605.

A lead wiring 608 is a wiring for transmitting signals to be input to the source line driver circuit 601 and the gate line driver circuit 603 and receives a video signal, a clock signal, a start signal, a reset signal, or the like from an FPC (flexible printed circuit) 609 serving as an external input terminal. Although only the FPC is illustrated here, a printed wiring board (PWB) may be attached to the FPC. The light-emitting apparatus in this specification includes, in its category, not only the light-emitting apparatus itself but also the light-emitting apparatus provided with the FPC or the PWB.

Next, a cross-sectional structure is described with reference to FIG. 2B. The driver circuit portions and the pixel portion are formed over an element substrate 610; here, the source line driver circuit 601, which is a driver circuit portion, and one pixel in the pixel portion 602 are illustrated.

The element substrate 610 may be formed using a substrate containing glass, quartz, an organic resin, a metal, an alloy, a semiconductor, or the like or a plastic substrate formed of FRP (Fiber Reinforced Plastics), PVF (poly(vinyl fluoride)), polyester, acrylic resin, or the like.

The structure of transistors used in pixels or driver circuits is not particularly limited. For example, inverted staggered transistors may be used, or staggered transistors may be used. Furthermore, top-gate transistors or bottom-gate transistors may be used. A semiconductor material used for the transistors is not particularly limited, and for example, silicon, germanium, silicon carbide, gallium nitride, or the like can be used. Alternatively, an oxide semiconductor containing at least one of indium, gallium, and zinc, such as an In—Ga—Zn-based metal oxide, may be used.

There is no particular limitation on the crystallinity of a semiconductor material used for the transistors, and an amorphous semiconductor or a semiconductor having crystallinity (a microcrystalline semiconductor, a polycrystalline semiconductor, a single crystal semiconductor, or a semiconductor partly including crystal regions) may be used. A semiconductor having crystallinity is preferably used because deterioration of the transistor characteristics can be inhibited.

Here, an oxide semiconductor is preferably used for semiconductor devices such as the transistors provided in the pixels or driver circuits and transistors used for touch sensors described later, and the like. In particular, an oxide semiconductor having a wider band gap than silicon is preferably used. When an oxide semiconductor having a wider band gap than silicon is used, the off-state current of the transistors can be reduced.

The oxide semiconductor preferably contains at least indium (In) or zinc (Zn). Further preferably, the oxide semiconductor contains an oxide represented by an In-M-Zn-based oxide (M represents a metal such as Al, Ti, Ga, Ge, Y, Zr, Sn, La, Ce, or Hf).

As a semiconductor layer, it is particularly preferable to use an oxide semiconductor film including a plurality of crystal parts whose c-axes are aligned perpendicular to a surface on which the semiconductor layer is formed or the top surface of the semiconductor layer and in which the adjacent crystal parts have no grain boundary.

The use of such materials for the semiconductor layer makes it possible to provide a highly reliable transistor in which a change in the electrical characteristics is inhibited.

Charge accumulated in a capacitor through a transistor including the above-described semiconductor layer can be held for a long time because of the low off-state current of the transistor. When such a transistor is used in a pixel, operation of a driver circuit can be stopped while a gray scale of an image displayed in each display region is maintained. As a result, an electronic apparatus with extremely low power consumption can be obtained.

For stable characteristics of the transistor, a base film is preferably provided. The base film can be formed with a single layer or stacked layers using an inorganic insulating film such as a silicon oxide film, a silicon nitride film, a silicon oxynitride film, or a silicon nitride oxide film. The base film can be formed by a sputtering method, a CVD (Chemical Vapor Deposition) method (e.g., a plasma CVD method, a thermal CVD method, or an MOCVD (Metal Organic CVD) method), an ALD (Atomic Layer Deposition) method, a coating method, a printing method, or the like. Note that the base film is not necessarily provided.

Note that an FET 623 is illustrated as a transistor formed in the driver circuit portion 601. The driver circuit may be formed with any of a variety of circuits such as a CMOS circuit, a PMOS circuit, or an NMOS circuit. Although a driver integrated type in which the driver circuit is formed over the substrate is illustrated in this embodiment, the driver circuit is not necessarily formed over the substrate, and the driver circuit can be formed outside, not over the substrate.

The pixel portion 602 includes a plurality of pixels including a switching FET 611, a current controlling FET 612, and an anode 613 electrically connected to a drain of the current controlling FET 612. One embodiment of the present invention is not limited to the structure, and a pixel portion in which three or more FETs and a capacitor are combined may be employed.

Note that an insulator 614 is formed to cover an end portion of the anode 613. Here, the insulator 614 can be formed using a positive photosensitive acrylic resin film.

In order to improve the coverage with an EL layer or the like which is formed later, the insulator 614 is formed to have a curved surface with curvature at its upper or lower end portion. For example, in the case where a positive photosensitive acrylic resin is used as a material of the insulator 614, only the upper end portion of the insulator 614 preferably has a curved surface with a curvature radius (0.2 μm to 3 μm). As the insulator 614, either a negative photosensitive resin or a positive photosensitive resin can be used.

An EL layer 616 and a cathode 617 are formed over the anode 613. Here, a material having a high work function is preferably used as a material of the anode 613. For example, a single-layer film of an ITO film, an indium tin oxide film containing silicon, an indium oxide film containing zinc oxide at 2 to 20 wt %, a titanium nitride film, a chromium film, a tungsten film, a Zn film, a Pt film, or the like, a stack of a titanium nitride film and a film containing aluminum as its main component, a stack of three layers of a titanium nitride film, a film containing aluminum as its main component, and a titanium nitride film, or the like can be used. The stacked-layer structure enables low wiring resistance, favorable ohmic contact, and a function as an anode.

The EL layer 616 is formed by any of a variety of methods such as an evaporation method using an evaporation mask, an inkjet method, and a spin coating method. The EL layer 616 has the structure described in Embodiment 1. As another material included in the EL layer 616, a low molecular compound or a high molecular compound (including an oligomer or a dendrimer) may be used.

As a material used for the cathode 617, which is formed over the EL layer 616, a material having a low work function (e.g., Al, Mg, Li, and Ca, or an alloy or a compound thereof, such as MgAg, MgIn, and AlLi) is preferably used. In the case where light generated in the EL layer 616 passes through the cathode 617, a stack of a thin metal film and a transparent conductive film (e.g., ITO, indium oxide containing zinc oxide at 2 to 20 wt %, an indium tin oxide containing silicon, or zinc oxide (ZnO)) is preferably used for the cathode 617.

Note that the light-emitting device is formed with the anode 613, the EL layer 616, and the cathode 617. The light-emitting device is the light-emitting device described in Embodiment 1. In the light-emitting apparatus of this embodiment, the pixel portion, which includes a plurality of light-emitting devices, may include both the light-emitting device described in Embodiment 1 and a light-emitting device having a different structure.

The sealing substrate 604 is attached to the element substrate 610 with the sealing material 605, so that a light-emitting device 618 is provided in the space 607 surrounded by the element substrate 610, the sealing substrate 604, and the sealing material 605. The space 607 may be filled with a filler, or may be filled with an inert gas (such as nitrogen or argon), or the sealing material. It is preferable that the sealing substrate be provided with a recessed portion and a drying agent be provided in the recessed portion, in which case deterioration due to influence of moisture can be inhibited.

An epoxy-based resin or glass frit is preferably used for the sealing material 605. It is preferable that such a material transmit moisture or oxygen as little as possible. As the sealing substrate 604, a glass substrate, a quartz substrate, or a plastic substrate formed of FRP (Fiber Reinforced Plastics), PVF (poly(vinyl fluoride)), polyester, acrylic resin, or the like can be used.

Although not illustrated in FIG. 2A and FIG. 2B, a protective film may be provided over the cathode. As the protective film, an organic resin film or an inorganic insulating film may be formed. The protective film may be formed so as to cover an exposed portion of the sealing material 605. The protective film may be provided so as to cover surfaces and side surfaces of the pair of substrates and exposed side surfaces of a sealing layer, an insulating layer, and the like.

The protective film can be formed using a material that does not easily transmit an impurity such as water. Thus, diffusion of an impurity such as water from the outside into the inside can be effectively inhibited.

As a material of the protective film, an oxide, a nitride, a fluoride, a sulfide, a ternary compound, a metal, a polymer, or the like can be used. For example, a material containing aluminum oxide, hafnium oxide, hafnium silicate, lanthanum oxide, silicon oxide, strontium titanate, tantalum oxide, titanium oxide, zinc oxide, niobium oxide, zirconium oxide, tin oxide, yttrium oxide, cerium oxide, scandium oxide, erbium oxide, vanadium oxide, indium oxide, or the like; a material containing aluminum nitride, hafnium nitride, silicon nitride, tantalum nitride, titanium nitride, niobium nitride, molybdenum nitride, zirconium nitride, gallium nitride, or the like; a material containing a nitride containing titanium and aluminum, an oxide containing titanium and aluminum, an oxide containing aluminum and zinc, a sulfide containing manganese and zinc, a sulfide containing cerium and strontium, an oxide containing erbium and aluminum, an oxide containing yttrium and zirconium, or the like can be used.

The protective film is preferably formed using a deposition method with favorable step coverage. One such method is an atomic layer deposition (ALD) method. A material that can be deposited by an ALD method is preferably used for the protective film. A dense protective film having reduced defects such as cracks or pinholes or a uniform thickness can be formed by an ALD method. Furthermore, damage caused to a process member in forming the protective film can be reduced.

By an ALD method, a uniform protective film with few defects can be formed even on, for example, a surface with a complex uneven shape or upper, side, and lower surfaces of a touch panel.

As described above, the light-emitting apparatus manufactured using the light-emitting device described in Embodiment 1 can be obtained.

The light-emitting apparatus in this embodiment is manufactured using the light-emitting device described in Embodiment 1 and thus can have favorable characteristics. Specifically, since the light-emitting device described in Embodiment 1 has favorable emission efficiency, the light-emitting apparatus can achieve low power consumption.

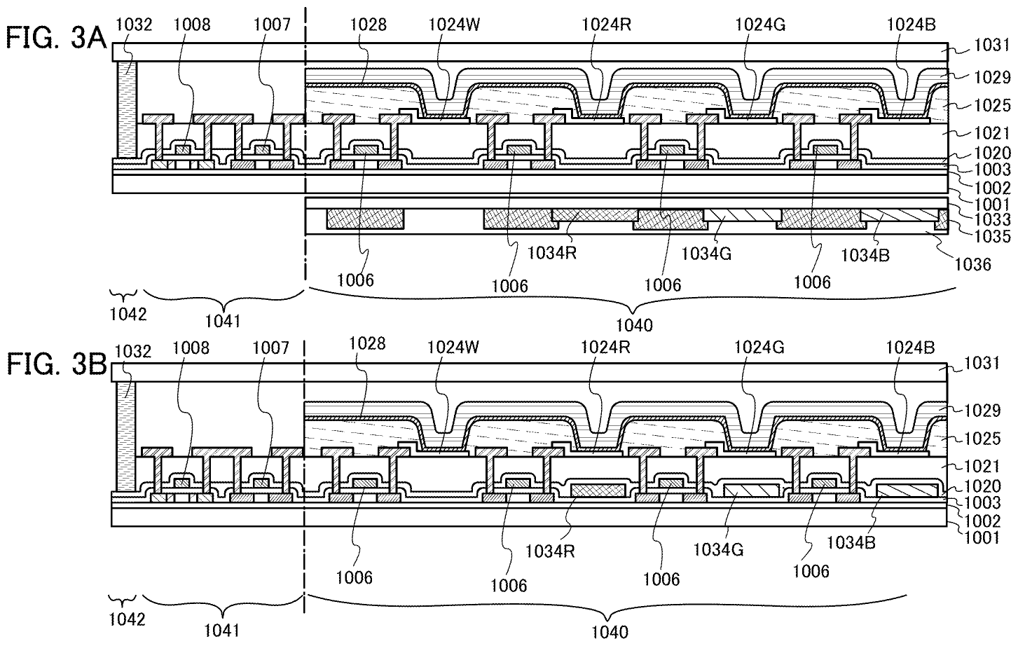

FIG. 3A and FIG. 3B each illustrate an example of a light-emitting apparatus that includes a light-emitting device exhibiting white light emission and coloring layers (color filters) and the like to display a full-color image. FIG. 3A illustrates a substrate 1001, a base insulating film 1002, a gate insulating film 1003, gate electrodes 1006, 1007, and 1008, a first interlayer insulating film 1020, a second interlayer insulating film 1021, a peripheral portion 1042, a pixel portion 1040, a driver circuit portion 1041, anodes 1024W, 1024R, 1024G, and 1024B of light-emitting devices, a partition 1025, an EL layer 1028, a cathode 1029 of the light-emitting devices, a sealing substrate 1031, a sealing material 1032, and the like.

In FIG. 3A, coloring layers (a red coloring layer 1034R, a green coloring layer 1034G, and a blue coloring layer 1034B) are provided on a transparent base material 1033. A black matrix 1035 may be additionally provided. The transparent base material 1033 provided with the coloring layers and the black matrix is aligned and fixed to the substrate 1001. Note that the coloring layers and the black matrix 1035 are covered with an overcoat layer 1036. In FIG. 3A, light emitted from part of the light-emitting layer does not pass through the coloring layers and is released to the outside, while light emitted from the other part of the light-emitting layer passes through the coloring layers and is released to the outside. The light that does not pass through the coloring layers is white and the light that passes through any one of the coloring layers is red, green, or blue; thus, an image can be expressed using pixels of the four colors.

FIG. 3B shows an example in which the coloring layers (the red coloring layer 1034R, the green coloring layer 1034G, and the blue coloring layer 1034B) are provided between the gate insulating film 1003 and the first interlayer insulating film 1020. As in the structure, the coloring layers may be provided between the substrate 1001 and the sealing substrate 1031.

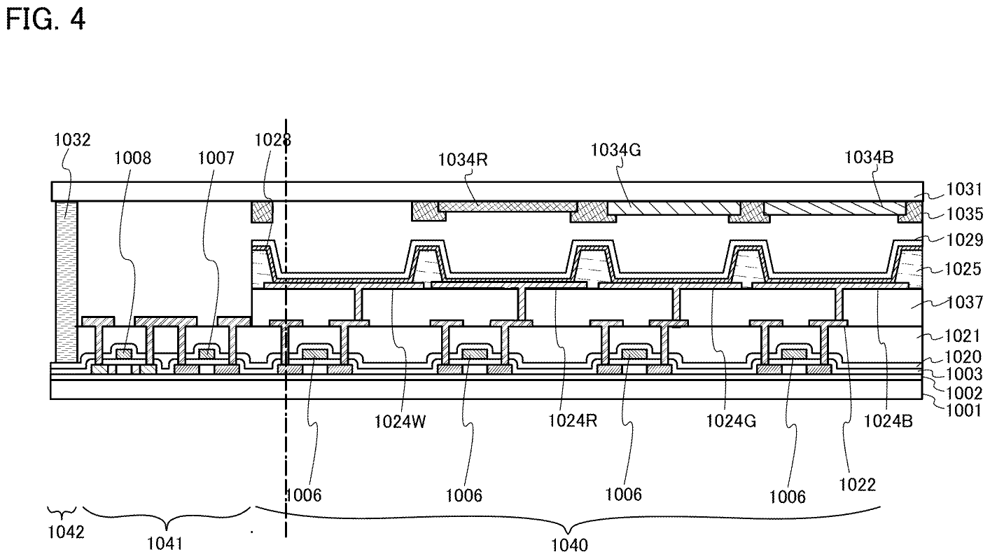

The above-described light-emitting apparatus has a structure in which light is extracted from the substrate 1001 side where FETs are formed (a bottom emission structure), but may have a structure in which light is extracted from the sealing substrate 1031 side (a top emission structure). FIG. 4 is a cross-sectional view of a light-emitting apparatus having a top emission structure. In this case, a substrate that does not transmit light can be used as the substrate 1001. The process up to the step of forming a connection electrode which connects the FET and the anode of the light-emitting device is performed in a manner similar to that of the light-emitting apparatus having a bottom emission structure. Then, a third interlayer insulating film 1037 is formed to cover the electrode 1022. This insulating film may have a planarization function. The third interlayer insulating film 1037 can be formed using a material similar to that of the second interlayer insulating film, and can alternatively be formed using any of other known materials.

The anodes 1024W, 1024R, 1024G, and 1024B of the light-emitting devices each serve as an anode here, but may serve as a cathode. Furthermore, in the case of a light-emitting apparatus having a top emission structure as illustrated in FIG. 4, the anodes are preferably reflective electrodes. The EL layer 1028 is formed to have a structure similar to the structure of the EL layer 103 described in Embodiment 1, with which white light emission can be obtained.

In the case of a top emission structure as illustrated in FIG. 4, sealing can be performed with the sealing substrate 1031 on which the coloring layers (the red coloring layer 1034R, the green coloring layer 1034G, and the blue coloring layer 1034B) are provided. The sealing substrate 1031 may be provided with the black matrix 1035 which is positioned between pixels. The coloring layers (the red coloring layer 1034R, the green coloring layer 1034G, and the blue coloring layer 1034B) or the black matrix may be covered with the overcoat layer 1036. Note that a light-transmitting substrate is used as the sealing substrate 1031. Although an example in which full color display is performed using four colors of red, green, blue, and white is shown here, there is no particular limitation and full color display using four colors of red, yellow, green, and blue or three colors of red, green, and blue may be performed.

In the light-emitting apparatus having a top emission structure, a microcavity structure can be favorably employed. A light-emitting device with a microcavity structure is formed with the use of a reflective electrode as the anode and a transflective electrode as the cathode. The light-emitting device with a microcavity structure includes at least an EL layer between the reflective electrode and the transflective electrode, and the EL layer includes at least a light-emitting layer serving as a light-emitting region.

Note that the reflective electrode has a visible light reflectivity of 40% to 100%, preferably 70% to 100%, and a resistivity of 1×10⁻² Ωcm or lower. In addition, the transflective electrode has a visible light reflectivity of 20% to 80%, preferably 40% to 70%, and a resistivity of 1×10⁻² Ωcm or lower.

Light emitted from the light-emitting layer included in the EL layer is reflected and resonated by the reflective electrode and the transflective electrode.

In the light-emitting device, by changing thicknesses of the transparent conductive film, the composite material, the carrier-transport material, or the like, the optical path length between the reflective electrode and the transflective electrode can be changed. Thus, light with a wavelength that is resonated between the reflective electrode and the transflective electrode can be intensified while light with a wavelength that is not resonated therebetween can be attenuated.

Note that light that is reflected back by the reflective electrode (first reflected light) considerably interferes with light that directly enters the transflective electrode from the light-emitting layer (first incident light). For this reason, the optical path length between the reflective electrode and the light-emitting layer is preferably adjusted to (2n−1)λ/4 (n is a natural number of 1 or larger and λ is a wavelength of light to be amplified). By adjusting the optical path length, the phases of the first reflected light and the first incident light can be aligned with each other and the light emitted from the light-emitting layer can be further amplified.

Note that in the above structure, the EL layer may include a plurality of light-emitting layers or may include a single light-emitting layer; for example, in combination with the structure of the above-described tandem light-emitting device, a plurality of EL layers each including a single or a plurality of light-emitting layer(s) may be provided in one light-emitting device with a charge-generation layer interposed between the EL layers.

With the microcavity structure, emission intensity with a specific wavelength in the front direction can be increased, whereby power consumption can be reduced. Note that in the case of a light-emitting apparatus that displays images with subpixels of four colors, red, yellow, green, and blue, the light-emitting apparatus can have favorable characteristics because the luminance can be increased owing to yellow light emission and each subpixel can employ a microcavity structure suitable for wavelengths of the corresponding color.

The light-emitting apparatus in this embodiment is manufactured using the light-emitting device described in Embodiment 1 and thus can have favorable characteristics. Specifically, since the light-emitting device described in Embodiment 1 has favorable emission efficiency, the light-emitting apparatus can achieve low power consumption.

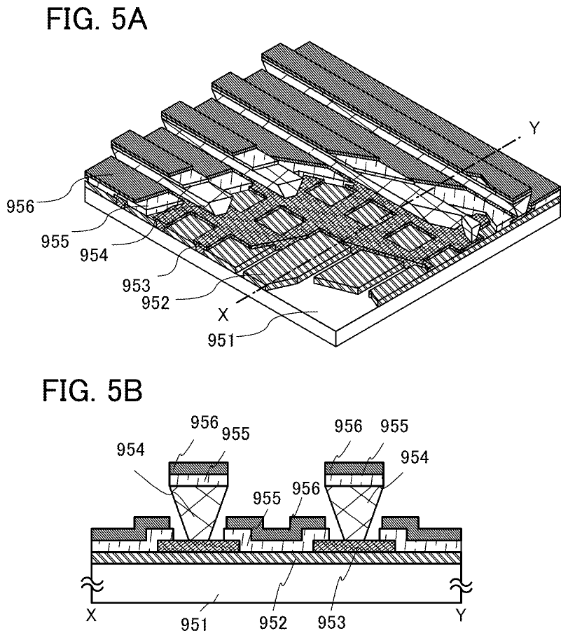

The active matrix light-emitting apparatus is described above, whereas a passive matrix light-emitting apparatus is described below. FIG. 5A and FIG. 5B illustrate a passive matrix light-emitting apparatus manufactured using the present invention. Note that FIG. 5A is a perspective view of the light-emitting apparatus, and FIG. 5B is a cross-sectional view taken along the dashed-dotted line X-Y in FIG. 5A. In FIG. 5, over a substrate 951, an EL layer 955 is provided between an electrode 952 and an electrode 956. An end portion of the electrode 952 is covered with an insulating layer 953. A partition layer 954 is provided over the insulating layer 953. The sidewalls of the partition layer 954 are aslope such that the distance between both sidewalls is gradually narrowed toward the surface of the substrate. In other words, a cross section taken along the direction of the short side of the partition layer 954 is trapezoidal, and the lower side (a side that is parallel to the surface of the insulating layer 953 and is in contact with the insulating layer 953) is shorter than the upper side (a side that is parallel to the surface of the insulating layer 953 and is not in contact with the insulating layer 953). The partition layer 954 thus provided can prevent defects in the light-emitting device due to static electricity or others. The passive matrix light-emitting apparatus also includes the light-emitting device described in Embodiment 1; thus, the light-emitting apparatus can have favorable reliability or low power consumption.

In the light-emitting apparatus described above, many minute light-emitting devices arranged in a matrix can each be controlled; thus, the light-emitting apparatus can be suitably used as a display device for displaying images.

This embodiment can be freely combined with any of the other embodiments.

### Embodiment 3

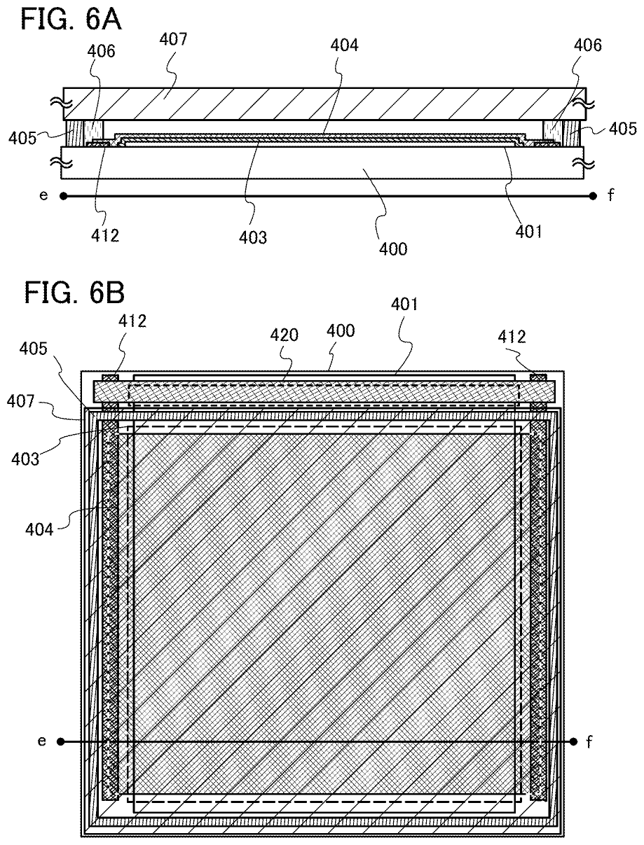

In this embodiment, an example in which the light-emitting device described in Embodiment 1 is used for a lighting device will be described with reference to FIG. 6. FIG. 6B is a top view of the lighting device, and FIG. 6A is a cross-sectional view taken along the line e-f in FIG. 6B.

In the lighting device in this embodiment, an anode 401 is formed over a substrate 400 which is a support with a light-transmitting property. The anode 401 corresponds to the anode 101 in Embodiment 1. When light is extracted through the anode 401, the anode 401 is formed using a material having a light-transmitting property.

A pad 412 for applying voltage to a cathode 404 is provided over the substrate 400.

An EL layer 403 is formed over the anode 401. The structure of the EL layer 403 corresponds to, for example, the structure of the EL layer 103 in Embodiment 1, or the structure in which light-emitting units 511 and 512 and a charge-generation layer 513 are combined. Refer to the descriptions for the structure.

The cathode 404 is formed to cover the EL layer 403. The cathode 404 corresponds to the cathode 102 in Embodiment 1. The cathode 404 is formed using a material having high reflectance when light is extracted through the anode 401. The cathode 404 is connected to the pad 412, thereby receiving voltage.

As described above, the lighting device described in this embodiment includes a light-emitting device including the anode 401, the EL layer 403, and the cathode 404. Since the light-emitting device is a light-emitting device with high emission efficiency, the lighting device in this embodiment can have low power consumption.

The substrate 400 provided with the light-emitting device having the above structure is fixed to a sealing substrate 407 with sealing materials 405 and 406 and sealing is performed, whereby the lighting device is completed. It is possible to use only either the sealing material 405 or the sealing material 406. The inner sealing material 406 (not illustrated in FIG. 6B) can be mixed with a desiccant which enables moisture to be adsorbed, increasing reliability.

When parts of the pad 412 and the anode 401 are extended to the outside of the sealing materials 405 and 406, the extended parts can serve as external input terminals. An IC chip 420 mounted with a converter or the like may be provided over the external input terminals.

The lighting device described in this embodiment includes as an EL element the light-emitting device described in Embodiment 1; thus, the lighting device can have low power consumption.

This embodiment can be freely combined with any of the other embodiments.

### Embodiment 4

In this embodiment, examples of electronic apparatuses each including the light-emitting device described in Embodiment 1 will be described. The light-emitting device described in Embodiment 1 has favorable emission efficiency and low power consumption. As a result, the electronic apparatuses described in this embodiment can be electronic apparatuses each including a light-emitting portion with low power consumption.

Examples of the electronic apparatus including the above light-emitting device include television devices (also referred to as TV or television receivers), monitors for computers and the like, digital cameras, digital video cameras, digital photo frames, cellular phones (also referred to as mobile phones or mobile phone devices), portable game machines, portable information terminals, audio playback devices, and large game machines such as pachinko machines. Specific examples of these electronic apparatuses are shown below.

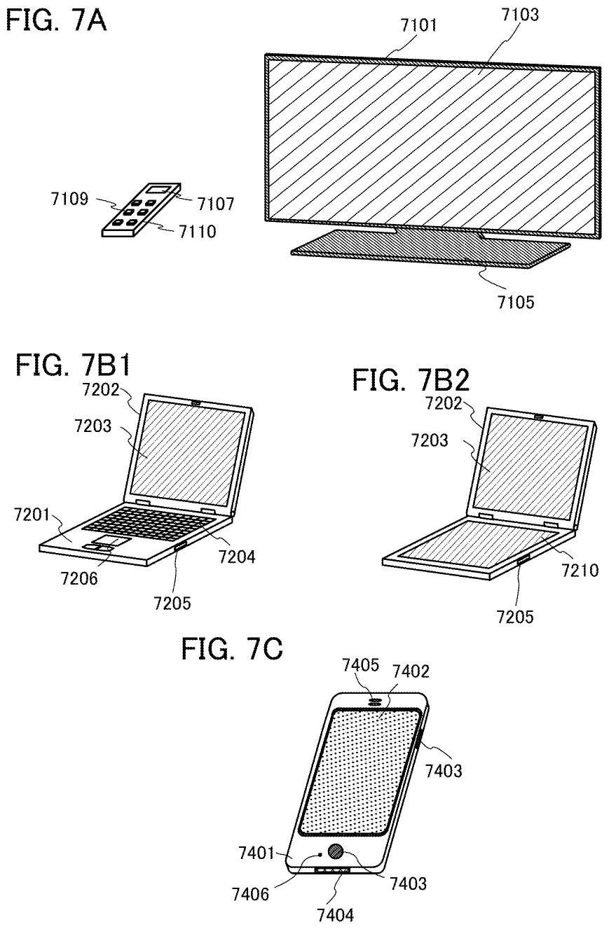

FIG. 7A shows an example of a television device. In the television device, a display portion 7103 is incorporated in a housing 7101. Here, the housing 7101 is supported by a stand 7105. Images can be displayed on the display portion 7103, and in the display portion 7103, the light-emitting devices described in Embodiment 1 are arranged in a matrix.

The television device can be operated with an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110. The light-emitting devices described in Embodiment 1 may also be arranged in a matrix in the display portion 7107.

Note that the television device is provided with a receiver, a modem, and the like. With the use of the receiver, a general television broadcast can be received, and moreover, when the television device is connected to a communication network with or without wires via the modem, one-way (from a sender to a receiver) or two-way (between a sender and a receiver or between receivers) data communication can be performed.

FIG. 7B1 illustrates a computer, which includes a main body 7201, a housing 7202, a display portion 7203, a keyboard 7204, an external connection port 7205, a pointing device 7206, and the like. Note that this computer is manufactured using the light-emitting devices described in Embodiment 1 and arranged in a matrix in the display portion 7203. The computer illustrated in FIG. 7B1 may have a structure illustrated in FIG. 7B2. A computer illustrated in FIG. 7B2 is provided with a display portion 7210 instead of the keyboard 7204 and the pointing device 7206. The display portion 7210 is a touch panel, and input operation can be performed by touching display for input on the display portion 7210 with a finger or a dedicated pen. The display portion 7210 can also display images other than the display for input. The display portion 7203 may also be a touch panel. Connecting the two screens with a hinge can prevent troubles; for example, the screens can be prevented from being cracked or broken while the computer is being stored or carried.

FIG. 7C shows an example of a portable terminal. A cellular phone is provided with a display portion 7402 incorporated in a housing 7401, operation buttons 7403, an external connection port 7404, a speaker 7405, a microphone 7406, and the like. Note that the cellular phone has the display portion 7402 in which the light-emitting devices described in Embodiment 1 are arranged in a matrix.

When the display portion 7402 of the portable terminal illustrated in FIG. 7C is touched with a finger or the like, data can be input to the portable terminal. In this case, operations such as making a call and creating an e-mail can be performed by touching the display portion 7402 with a finger or the like.

The display portion 7402 has mainly three screen modes. The first mode is a display mode mainly for displaying images. The second mode is an input mode mainly for inputting data such as text. The third mode is a display-and-input mode in which the two modes, the display mode and the input mode, are combined.

For example, in the case of making a call or creating an e-mail, a text input mode mainly for inputting text is selected for the display portion 7402 so that text displayed on the screen can be input. In this case, it is preferable to display a keyboard or number buttons on almost the entire screen of the display portion 7402.

When a sensing device including a sensor such as a gyroscope sensor or an acceleration sensor for detecting inclination is provided inside the portable terminal, display on the screen of the display portion 7402 can be automatically changed by determining the orientation of the portable terminal (whether the portable terminal is placed horizontally or vertically).

The screen modes are switched by touching the display portion 7402 or operating the operation buttons 7403 of the housing 7401. Alternatively, the screen modes can be switched depending on the kind of images displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, when input by touching the display portion 7402 is not performed for a certain period while a signal sensed by an optical sensor in the display portion 7402 is sensed, the screen mode may be controlled so as to be switched from the input mode to the display mode.

The display portion 7402 may also function as an image sensor. For example, an image of a palm print, a fingerprint, or the like is taken when the display portion 7402 is touched with the palm or the finger, whereby personal authentication can be performed. Furthermore, by providing a backlight or a sensing light source which emits near-infrared light in the display portion, an image of a finger vein, a palm vein, or the like can be taken.

Note that the structure described in this embodiment can be combined with any of the structures described in Embodiment 1 to Embodiment 3 as appropriate.

As described above, the application range of the light-emitting apparatus including the light-emitting device described in Embodiment 1 is so wide that this light-emitting apparatus can be used in electronic apparatuses in a variety of fields. By using the light-emitting device described in Embodiment 1, an electronic apparatus with low power consumption can be obtained.

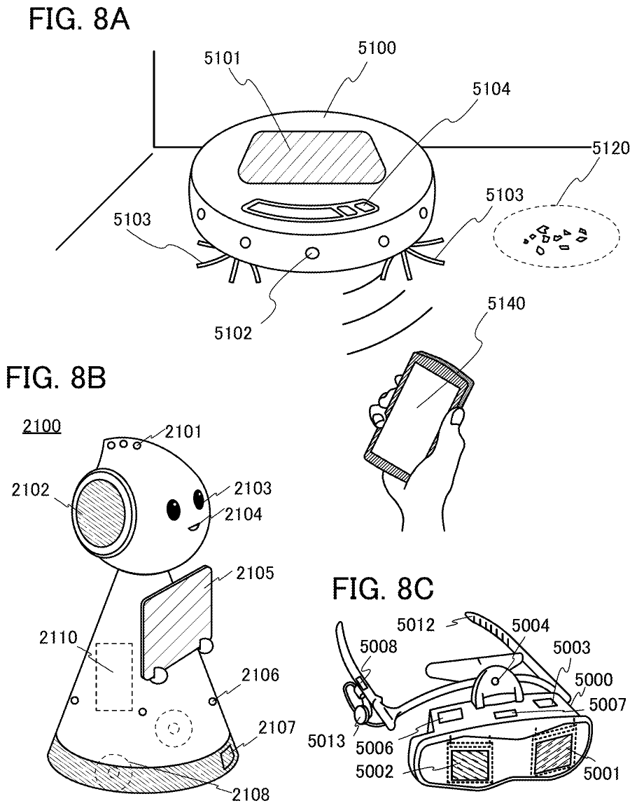

FIG. 8A is a schematic view showing an example of a cleaning robot.

A cleaning robot 5100 includes a display 5101 on its top surface, a plurality of cameras 5102 on its side surface, a brush 5103, and operation buttons 5104. Although not illustrated, the bottom surface of the cleaning robot 5100 is provided with a tire, an inlet, and the like. Furthermore, the cleaning robot 5100 includes various sensors such as an infrared sensor, an ultrasonic sensor, an acceleration sensor, a piezoelectric sensor, an optical sensor, and a gyroscope sensor. The cleaning robot 5100 has a wireless communication means.

The cleaning robot 5100 is self-propelled, detects dust 5120, and vacuums the dust through the inlet provided on the bottom surface.

The cleaning robot 5100 can determine whether there is an obstacle such as a wall, furniture, or a step by analyzing images taken by the cameras 5102. When an object that is likely to be caught in the brush 5103, such as a wire, is detected by image analysis, the rotation of the brush 5103 can be stopped.

The display 5101 can display the remaining capacity of a battery, the amount of vacuumed dust, and the like. The display 5101 may display a path on which the cleaning robot 5100 has run. The display 5101 may be a touch panel, and the operation buttons 5104 may be provided on the display 5101.

The cleaning robot 5100 can communicate with a portable electronic apparatus 5140 such as a smartphone. Images taken by the cameras 5102 can be displayed on the portable electronic apparatus 5140. Accordingly, an owner of the cleaning robot 5100 can monitor his/her room even when the owner is not at home. The owner can also check the display on the display 5101 by the portable electronic apparatus such as a smartphone.

The light-emitting apparatus of one embodiment of the present invention can be used for the display 5101.

A robot 2100 illustrated in FIG. 8B includes an arithmetic device 2110, an illuminance sensor 2101, a microphone 2102, an upper camera 2103, a speaker 2104, a display 2105, a lower camera 2106, an obstacle s sensor 2107, and a moving mechanism 2108.

The microphone 2102 has a function of detecting a speaking voice of a user, an environmental sound, and the like. The speaker 2104 has a function of outputting sound. The robot 2100 can communicate with a user using the microphone 2102 and the speaker 2104.

The display 2105 has a function of displaying various kinds of information. The robot 2100 can display information desired by a user on the display 2105. The display 2105 may be provided with a touch panel. Moreover, the display 2105 may be a detachable information terminal, in which case charging and data communication can be performed when the display 2105 is set at the home position of the robot 2100.

The upper camera 2103 and the lower camera 2106 each have a function of taking an image of the surroundings of the robot 2100. The obstacle sensor 2107 can detect an obstacle in the direction where the robot 2100 advances with the moving mechanism 2108. The robot 2100 can move safely by recognizing the surroundings with the upper camera 2103, the lower camera 2106, and the obstacle sensor 2107. The light-emitting apparatus of one embodiment of the present invention can be used for the display 2105.

FIG. 8C shows an example of a goggle-type display. The goggle-type display includes, for example, a housing 5000, a display portion 5001, a speaker 5003, an LED lamp 5004, a connection terminal 5006, a sensor 5007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared ray), a microphone 5008, a display portion 5002, a support portion 5012, and an earphone 5013.

The light-emitting apparatus of one embodiment of the present invention can be used for the display portion 5001 and the display portion 5002.

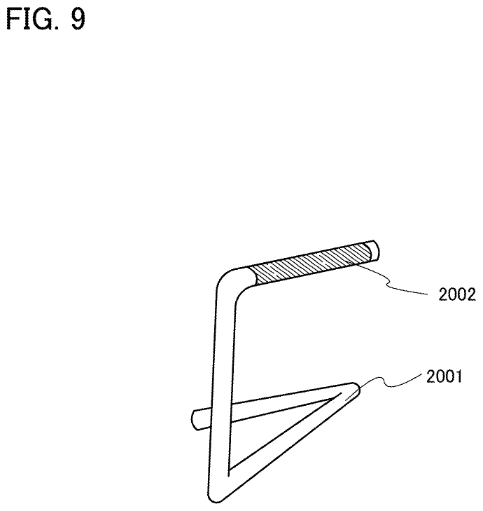

FIG. 9 shows an example in which the light-emitting device described in Embodiment 1 is used for a table lamp which is a lighting device. The table lamp illustrated in FIG. 9 includes a housing 2001 and a light source 2002, and the lighting device described in Embodiment 3 may be used for the light source 2002.

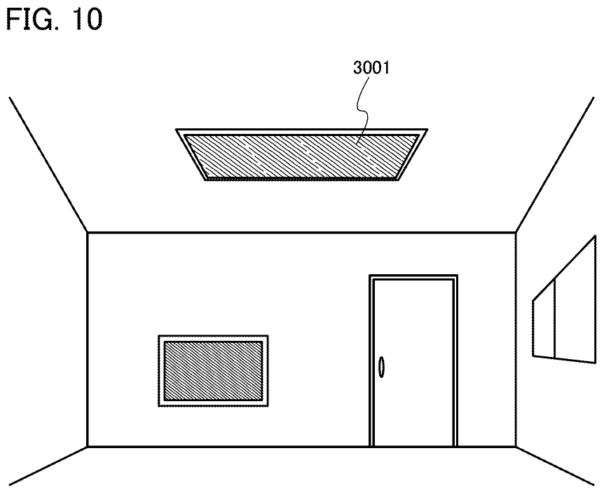

FIG. 10 shows an example in which the light-emitting device described in Embodiment 1 is used for an indoor lighting device 3001. Since the light-emitting device described in Embodiment 1 has high emission efficiency, the lighting device can have low power consumption. Furthermore, since the light-emitting device described in Embodiment 1 can have a large area, the light-emitting device can be used for a large-area lighting device. Furthermore, since the light-emitting device described in Embodiment 1 is thin, the light-emitting device can be used for a lighting device having a reduced thickness.

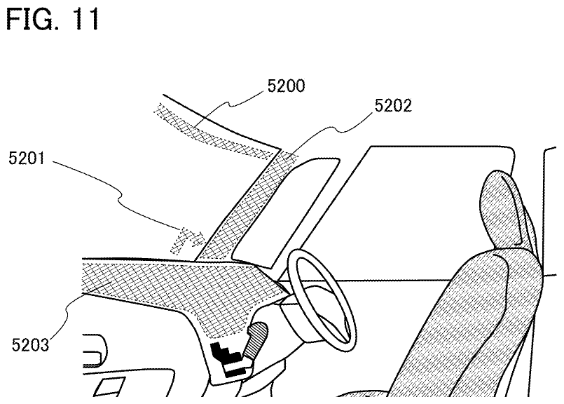

The light-emitting device described in Embodiment 1 can also be used for an automobile windshield or an automobile dashboard. FIG. 11 illustrates a mode in which the light-emitting devices described in Embodiment 1 are used for an automobile windshield and an automobile dashboard. A display region 5200 to a display region 5203 each include the light-emitting device described in Embodiment 1.

The display region 5200 and the display region 5201 are display devices which are provided in the automobile windshield and include the light-emitting device described in Embodiment 1. The light-emitting device described in Embodiment 1 can be formed into what is called a see-through display device, through which the opposite side can be seen, by including an anode and a cathode formed of light-transmitting electrodes. Such see-through display devices can be provided even in the automobile windshield without hindering the view. In the case where a driving transistor or the like is provided, a transistor having a light-transmitting property, such as an organic transistor including an organic semiconductor material or a transistor including an oxide semiconductor, is preferably used.

The display region 5202 is a display device which is provided in a pillar portion and includes the light-emitting device described in Embodiment 1. The display region 5202 can compensate for the view hindered by the pillar by displaying an image taken by an imaging unit provided in the car body. Similarly, the display region 5203 provided in the dashboard portion can compensate for the view hindered by the car body by displaying an image taken by an imaging unit provided on the outside of the automobile; thus, blind areas can be eliminated to enhance the safety. Images that compensate for the areas that a driver cannot see enable the driver to ensure safety easily and comfortably.

The display region 5203 can provide a variety of kinds of information such as navigation data, a speedometer, a tachometer, air-condition setting, and the like. The content or layout of the display can be changed as appropriate according to the user's preference. Note that such information can also be displayed on the display region 5200 to the display region 5202. The display region 5200 to the display region 5203 can also be used as lighting devices.

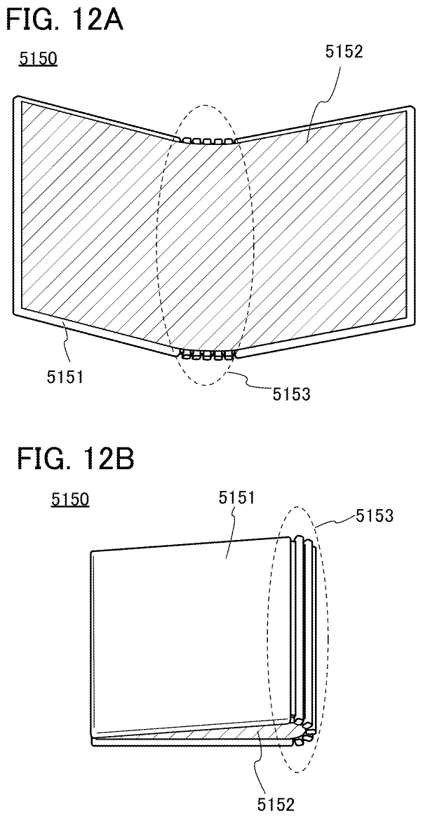

FIG. 12A and FIG. 12B illustrate a foldable portable information terminal 5150. The foldable portable information terminal 5150 includes a housing 5151, a display region 5152, and a bend portion 5153. FIG. 12A illustrates the portable information terminal 5150 that is opened. FIG. 12B illustrates the portable information terminal that is folded. Despite its large display region 5152, the portable information terminal 5150 is compact in size and has excellent portability when folded.

The display region 5152 can be folded in half with the bend portion 5153. The bend portion 5153 includes a flexible member and a plurality of supporting members. When the display region is folded, the flexible member expands. The bend portion 5153 has a radius of curvature greater than or equal to 2 mm, preferably greater than or equal to 3 mm.

Note that the display region 5152 may be a touch panel (an input/output device)including a touch sensor (an input device). The light-emitting apparatus of one embodiment of the present invention can be used for the display region 5152.

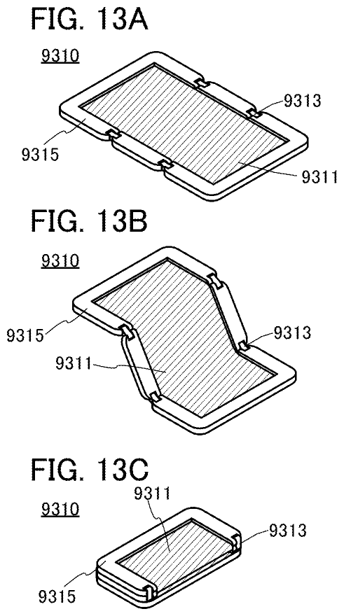

FIG. 13A to FIG. 13C illustrate a foldable portable information terminal 9310. FIG. 13A illustrates the portable information terminal 9310 that is opened. FIG. 13B illustrates the portable information terminal 9310 on the way from either the opened state or the folded state to the other state. FIG. 13C illustrates the portable information terminal 9310 that is folded. The portable information terminal 9310 is excellent in portability when folded, and is excellent in display browsability when opened because of a seamless large display region.

A display panel 9311 is supported by three housings 9315 joined together by hinges 9313. Note that the display panel 9311 may be a touch panel (an input/output device) including a touch sensor (an input device). By folding the display panel 9311 at the hinges 9313 between two housings 9315, the portable information terminal 9310 can be reversibly changed in shape from the opened state to the folded state. The light-emitting apparatus of one embodiment of the present invention can be used for the display panel 9311.

### Example 1

In this example, a light-emitting device 1 and a light-emitting device 2, each of which is one embodiment of the present invention described in the embodiment, and a comparative light-emitting device 1 to a comparative light-emitting device 3 are described. Structural formulae of organic compounds used in this example are shown below.

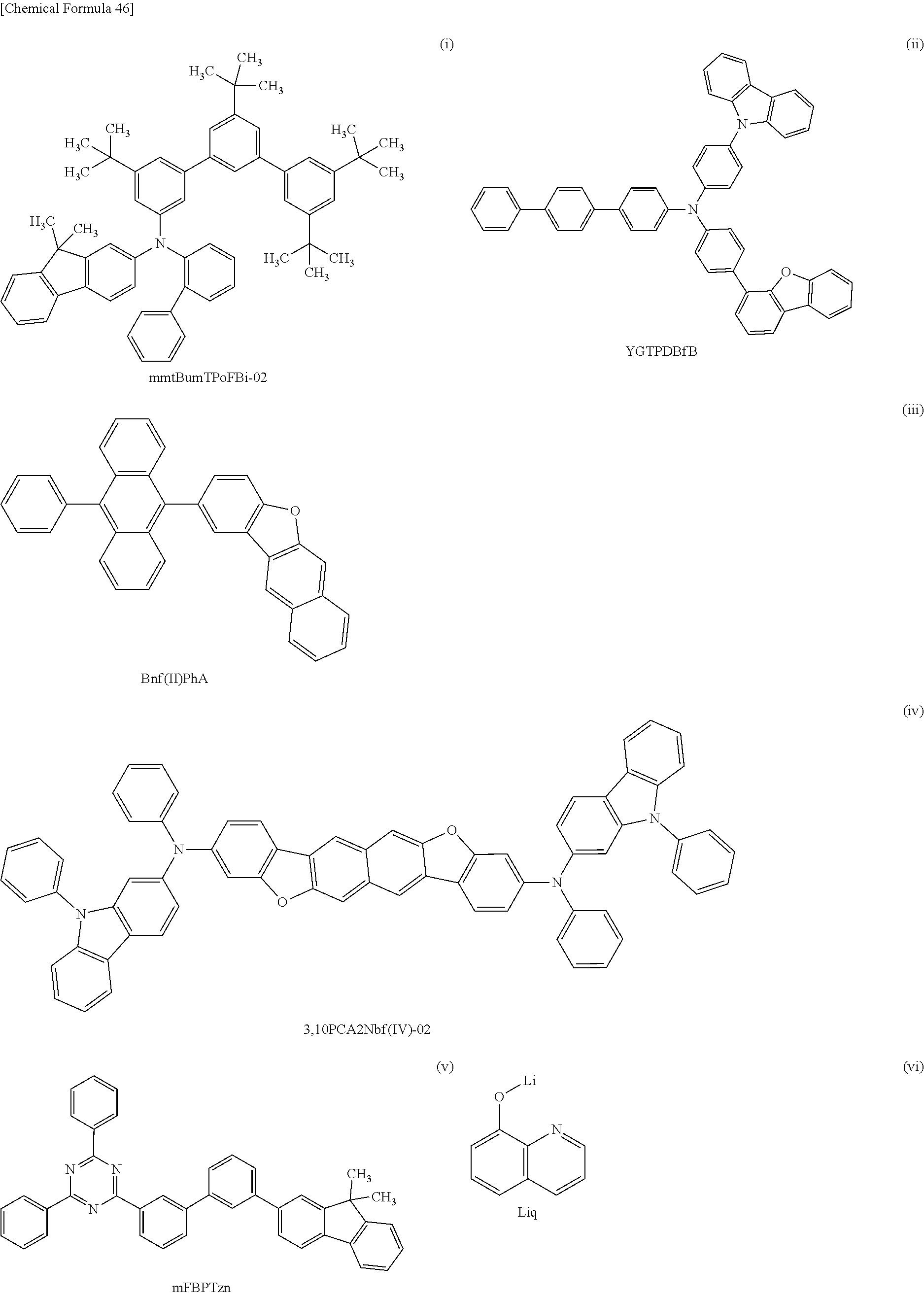

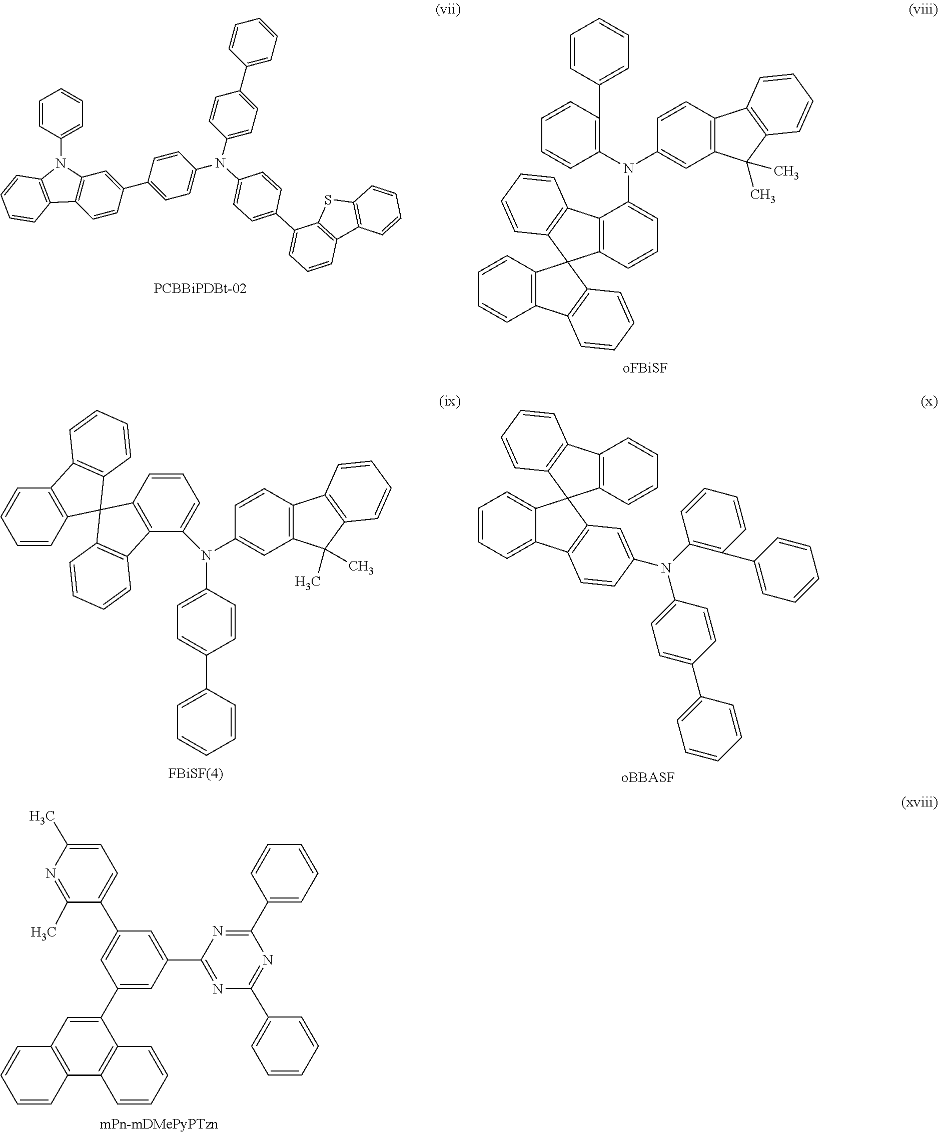 [SMILES: c1ccc(-c2ccc(N(c3ccc(-c4ccc5c6ccccc6n(-c6ccccc6)c5c4)cc3)c3ccc(-c4cccc5c4sc4ccccc45)cc3)cc2)cc1 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2ccc(N(c3ccc(-c4ccccc4)cc3)c3cccc4c3-c3ccccc3C43c4ccccc4-c4ccccc43)cc21 ; c1ccc(-c2ccc(N(c3ccc4c(c3)C3(c5ccccc5-c5ccccc53)c3ccccc3-4)c3ccccc3-c3ccccc3)cc2)cc1 ; Cc1ccc(-c2cc(-c3nc(-c4ccccc4)nc(-c4ccccc4)n3)cc(-c3cc4ccccc4c4ccccc34)c2)c(C)n1]

### (Fabrication Method of Light-Emitting Device 1)

First, as a transparent electrode, an indium tin oxide containing silicon oxide (ITSO) was deposited over a glass substrate to a thickness of 55 nm by a sputtering method, whereby the anode 101 was formed. The electrode area was set to 4 mm² (2 mm×2 mm).

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Next, the substrate provided with the anode 101 was fixed to a substrate holder provided in the vacuum evaporation apparatus such that the surface on which the anode 101 was formed faced downward, and N-(1,1′-biphenyl-2-yl)-N-(3,3″,5′,5″-tetra-t-butyl-1,1′: 3′,1″-terphenyl-5-yl)-9,9-dimethyl-9H-fluoren-2-amine (abbreviation: mmtBumTPoFBi-02) represented by Structural Formula (i) above and an electron acceptor material (OCHD-001) were deposited by co-evaporation on the anode 101 to 10 nm by an evaporation method such that the weight ratio was 1:0.1 (=mmtBumTPoFBi-02:OCHD-001), whereby the hole-injection layer 111 was formed.

Over the hole-injection layer 111, mmtBumTPoFBi-02 was deposited to 140 nm by evaporation, whereby the hole-transport layer 112 was formed.

Subsequently, over the hole-transport layer 112, N-[4-(9Hcarbazol-9-yl)phenyl]-N-[4-(4-dibenzofuranyl)phenyl]-[1,1′:4′,1″-terphenyl]-4-amine(abbreviation: YGTPDBfB) represented by Structural Formula (ii) above was deposited to 10 nm by evaporation, whereby an electron-blocking layer was formed.

Then, 2-(10-phenyl-9-anthracenyl)-benzo[b]naphtho[2,3-d]furan (abbreviation: Bnf(II)PhA) represented by Structural Formula (iii) above and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iv) above were deposited to 25 nm by co-evaporation such that the weight ratio was 1:0.015 (=Bnf(II)PhA:3,10PCA2Nbf(IV)-02), whereby the light-emitting layer 113 was formed.

After that, 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (v) above was deposited to nm by evaporation, whereby a hole-blocking layer was formed. Furthermore, 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (xviii) above and 8-quinolinolato-lithium (abbreviation: Liq) represented by Structural Formula (vi) above were deposited to 20 nm by co-evaporation such that the weight ratio was 1:1 (=mPn-mDMePyPTzn:Liq), whereby the electron-transport layer 114 was formed.

After the electron-transport layer 114 was formed, Liq was deposited to 1 nm to form the electron-injection layer 115, and lastly aluminum was deposited to a thickness of 200 nm by evaporation to form the cathode 102, whereby the light-emitting device 1 was fabricated.

### (Fabrication Method of Light-Emitting Device 2)

The light-emitting device 2 was fabricated in a manner similar to that of the light-emitting device 1 except that YGTPDBfB in the electron-blocking layer of the light-emitting device 1 was replaced with 4-(dibenzothiophen-4-yl)-4′-phenyl-4″-(9-phenyl-9H-carbazol-2-yl)triphenylamine (abbreviation: PCBBiPDBt-02) represented by Structural Formula (vii) above.

### (Fabrication Method of Comparative Light-Emitting Device 1)

The comparative light-emitting device 1 was fabricated in a manner similar to that of the light-emitting device 1 except that YGTPDBfB in the electron-blocking layer of the light-emitting device 1 was replaced with N-(1,1′-biphenyl-2-yl)-N-(9,9-dimethyl-9H-fluoren-2-yl)-9,9′-spirobi(9H-fluoren)-4-amine (abbreviation: oFBiSF) represented by Structural Formula (viii) above.

### (Fabrication Method of Comparative Light-Emitting Device 2)

The comparative light-emitting device 2 was fabricated in a manner similar to that of the light-emitting device 1 except that YGTPDBfB in the electron-blocking layer of the light-emitting device 1 was replaced with N-[1,1′-biphenyl]-4-yl-N-(9,9-dimethyl-9H-fluoren-2-yl)-9,9′-spirobi[9H-fluoren]-4-amine (abbreviation: FBiSF(4)) represented by Structural Formula (ix) above.

### (Fabrication Method of Comparative Light-Emitting Device 3)

The comparative light-emitting device 3 was fabricated in a manner similar to that of the light-emitting device 1 except that YGTPDBfB in the electron-blocking layer of the light-emitting device 1 was replaced with N-(1,1′-biphenyl-4-yl)-N-(1,1′-biphenyl-2-yl)-9,9′-spirobi(9H-fluorene)-2-amine (abbreviation: oBBASF) represented by Structural Formula (x) above.

The element structures of the light-emitting device 1, the light-emitting device 2, and the comparative light-emitting device 1 to the comparative light-emitting device 3 are listed in the following table.

TABLE 1

| | | | | | Comparative | Comparative | Comparative |
|---|---|---|---|---|---|---|---|
| | | | Light-emitting | Light-emitting | light-emitting | light-emitting | light-emitting |
| | | | device 1 | device 2 | device 1 | device 2 | device 3 |
| Electron-injection layer | 1 | nm | Liq | | | | |
| Electron-transport layer | 20 | nm | mPn-mDMePyPTzn:Liq | | | | |
| | | | (1:1) | | | | |
| Hole-blocking layer | 10 | nm | mFBPTzn | | | | |
| Light-emitting layer | 25 | nm | Bnf(II)PhA:3,10PCA2Nbf(IV)-02 | | | | |
| | | | (1:0.015) | | | | |
| Electron-blocking layer | 10 | nm | YGTPDBfB | PCBBiPDBt-02 | oFBiSF | FBiSF(4) | oBBASF |

| | | | |
|---|---|---|---|
| Hole-transport layer | 140 | nm | mmtBumTPoFBi-02 |
| Hole-injection layer | 10 | nm | mmtBumTPoFBi-02:OCHD-001 |
| | | | (1:0.1) |

Note that mmtBumTPoFBi-02 is a hole-transport material with a low refractive index: the ordinary refractive index in the entire blue light emission region (greater than or equal to 455 nm and less than or equal to 465 nm) is higher than or equal to 1.69 and lower than or equal to 1.70, which is within the range higher than or equal to 1.50 and lower than or equal to 1.75, and the ordinary refractive index at 633 nm is 1.64, which is within the range higher than or equal to 1.45 and lower than or equal to 1.70. The ordinary refractive index of Bnf(II)PhA in the entire blue light emission region (greater than or equal to 455 nm and less than or equal to 465 nm) is higher than or equal to 1.89 and lower than or equal to 1.91, and the ordinary refractive index at 633 nm is 1.79. Since the concentration of 3,10PCA2Nbf(IV)-02 in the light-emitting layer 113 is low, the refractive index of the light-emitting layer 113 is equivalent to that of Bnf(II)PhA. Accordingly, the light-emitting device of one embodiment of the present invention is a light-emitting device in which the refractive index of the hole-transport layer 112 is lower than that of the light-emitting layer 113.

Furthermore, YGTPDBfB and PCBBiPDBt-02 used in the electron-blocking layers of the light-emitting device 1 and the light-emitting device 2 are each a monoamine compound with a triarylamine structure having a group including a carbazole structure, a group including a dibenzofuran structure or a dibenzothiophene structure, and a group including an aromatic hydrocarbon structure having 6 to 18 carbon atoms. Note that three materials used for the electron-blocking layers of the comparative light-emitting devices 1 to 3 are each an organic compound that does not have the above structure.

The light-emitting devices and the comparative light-emitting devices were sealed using a glass substrate in a glove box containing a nitrogen atmosphere so as not to be exposed to the air (a UV curable sealing material was applied to surround the elements, only the sealing material was irradiated with UV while the light-emitting devices were not irradiated with the UV, and heat treatment was performed at 80° C. under an atmospheric pressure for one hour), and then the initial characteristics of the light-emitting devices were measured.

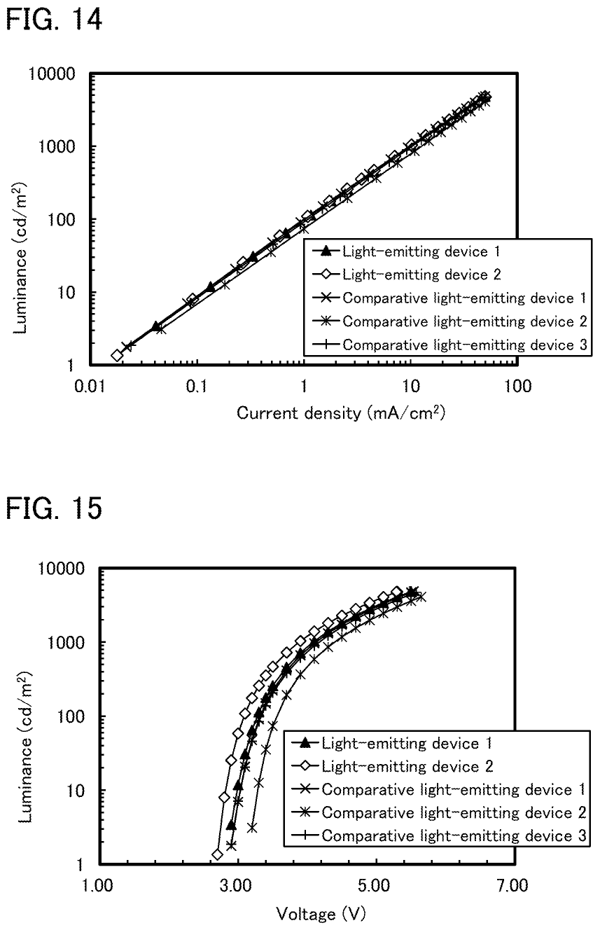

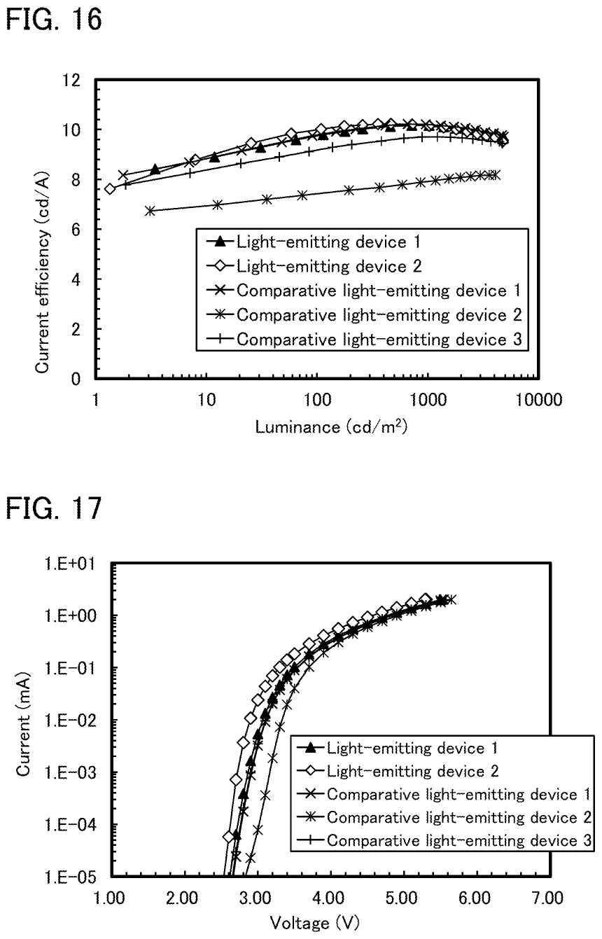

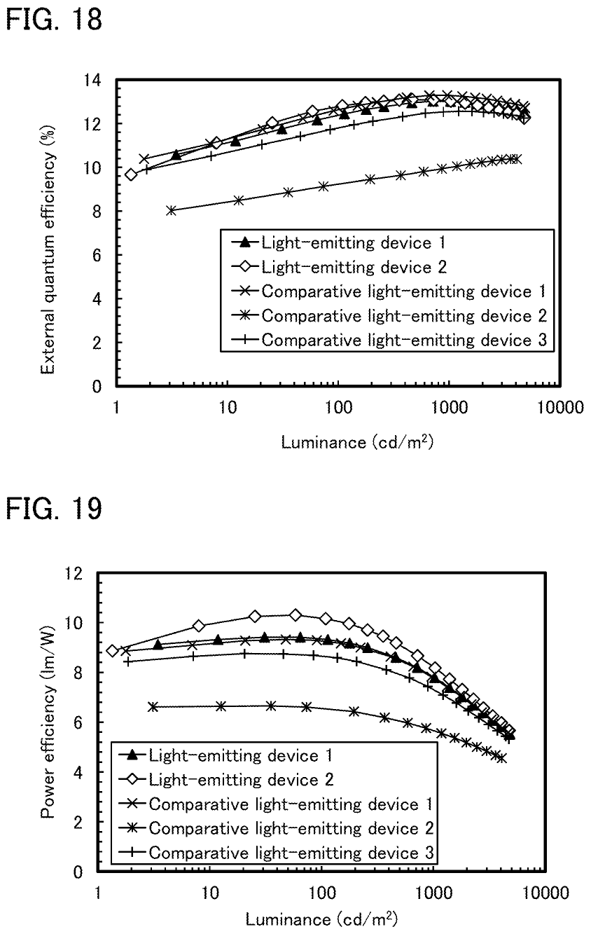

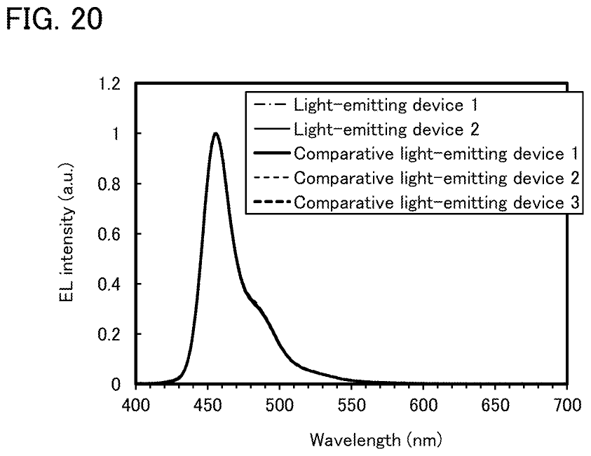

FIG. 14 shows the luminance-current density characteristics of the light-emitting device 1, the light-emitting device 2, and the comparative light-emitting device 1 to the comparative light-emitting device 3; FIG. 15 shows the luminance-voltage characteristics thereof, FIG. 16 shows the current efficiency-luminance characteristics thereof, FIG. 17 shows the current-voltage characteristics thereof, FIG. 18 shows the external quantum efficiency-luminance characteristics thereof, FIG. 19 shows the power efficiency-luminance characteristics thereof, and FIG. 20 shows the emission spectra thereof. Table 2 shows the main characteristics of the light-emitting device 1, the light-emitting device 2, and the comparative light-emitting device 1 to the comparative light-emitting device 3 at approximately 1000 cd/m². The luminance, CIE chromaticity, and emission spectra were measured at normal temperature with a spectroradiometer (SR-UL1R manufactured by TOPCON TECHNOHOUSE CORPORATION).

TABLE 2

| | | | Current | | Current | External |
|---|---|---|---|---|---|---|
| | Voltage | Current | density | Chromaticity | efficiency | quantum |
| | (V) | (mA) | (mA/cm²) | (x, y) | (cd/A) | efficiency (%) |
| Light-emitting device 1 | 4.1 | 0.40 | 10.1 | (0.140, 0.083) | 10.2 | 13.0 |
| Light-emitting device 2 | 3.9 | 0.41 | 10.2 | (0.140, 0.083) | 10.1 | 13.0 |
| Comparative light-emitting | 4.1 | 0.38 | 9.4 | (0.141, 0.081) | 10.2 | 13.3 |
| device 1 | | | | | | |
| Comparative light-emitting | 4.3 | 0.44 | 10.9 | (0.141, 0.085) | 7.9 | 9.9 |
| device 2 | | | | | | |
| Comparative light-emitting | 4.1 | 0.37 | 9.2 | (0.141, 0.082) | 9.7 | 12.5 |
| device 3 | | | | | | |

It was found from FIG. 14 to FIG. 20 and Table 2 that the light-emitting device 1 and the light-emitting device 2, each of which was one embodiment of the present invention, and the comparative light-emitting device 1 were light-emitting devices with favorable external quantum efficiency. In particular, the light-emitting device 2 had a low driving voltage and was, as a result, a light-emitting device with extremely favorable power efficiency.

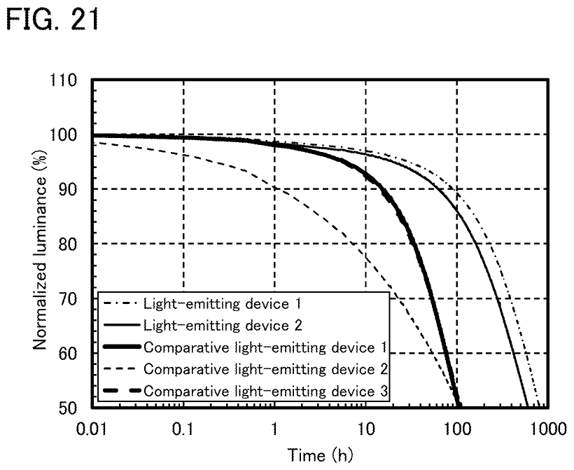

FIG. 21 is a graph showing a change in luminance over driving time at a current density of 50 mA/cm². As shown in FIG. 21, the light-emitting device 1 and the light-emitting device 2 were both found to be light-emitting devices with a favorable lifetime. Meanwhile, the comparative light-emitting devices 1 to 3 were found to be light-emitting devices that deteriorated faster than the light-emitting devices 1 and 2.

As described above, a light-emitting device in which a layer containing a monoamine compound having a specific structure is formed in contact with a low refractive index layer can be a light-emitting device with favorable characteristics. Specifically, the light-emitting device can have a favorable lifetime. Alternatively, the light-emitting device can have favorable emission efficiency. Alternatively, the light-emitting device can have a low driving voltage.

### Example 2

This example shows results obtained by examining the ease of carrier (hole in this case) flow in the stacked-layer structure formed between the anode and the light-emitting layer of the light-emitting device of one embodiment of the present invention described in the embodiment. Measurement devices (hole-only elements) in which only holes flowed were fabricated for measurement. A device 3 and a device 4 are measurement devices each of which has part of the stacked-layer structure of one embodiment of the present invention, and comparative devices 4 to 7 are measurement devices each of which does not have part of the stacked-layer structure of one embodiment of the present invention.

Structural formulae of organic compounds used for the device 3, the device 4, and the comparative devices 4 to 7 are shown below.

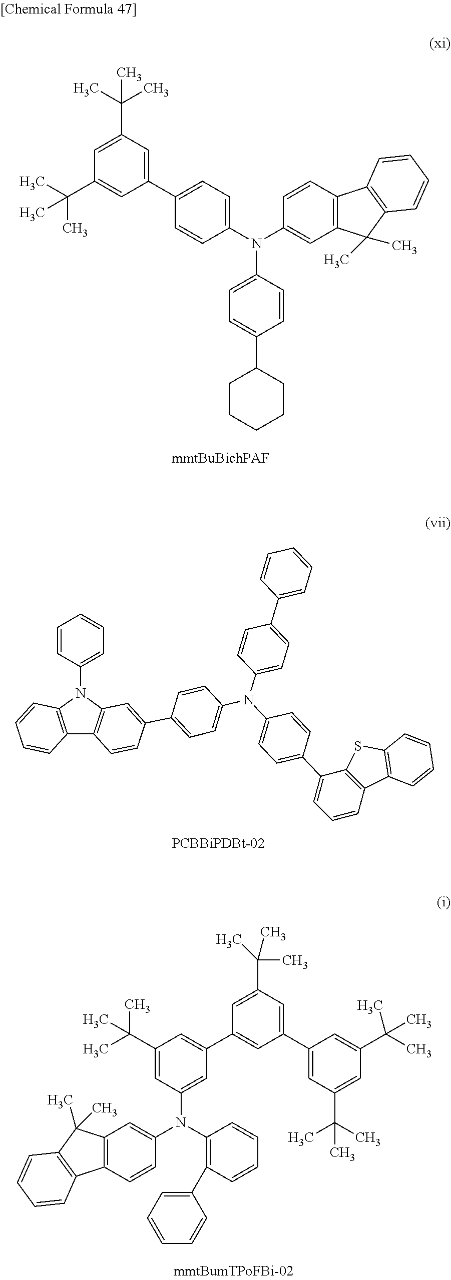 [SMILES: CC(C)(C)c1cc(-c2ccc(N(c3ccc(C4CCCCC4)cc3)c3ccc4c(c3)C(C)(C)c3ccccc3-4)cc2)cc(C(C)(C)C)c1 ; c1ccc(-c2ccc(N(c3ccc(-c4ccc5c6ccccc6n(-c6ccccc6)c5c4)cc3)c3ccc(-c4cccc5c4sc4ccccc45)cc3)cc2)cc1 ; CC(C)(C)c1cc(-c2cc(N(c3ccc4c(c3)C(C)(C)c3ccccc3-4)c3ccccc3-c3ccccc3)cc(C(C)(C)C)c2)cc(-c2cc(C(C)(C)C)cc(C(C)(C)C)c2)c1]

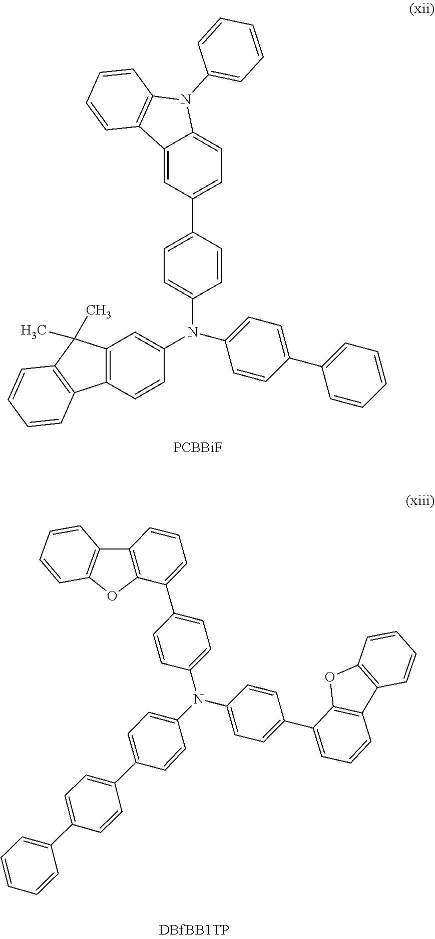

### (Fabrication Method of Device 3)

First, a silver-palladium-copper alloy (also referred to as APC) film was formed over a glass substrate to 100 nm, and then an indium tin oxide containing silicon oxide (ITSO) was deposited to a thickness of 45 nm by a sputtering method, whereby the anode was formed. The electrode area was set to 4 mm² (2 mm×2 mm).

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus where the pressure was reduced to approximately 104 Pa, vacuum baking was performed at 170° C. for 30 minutes in a heating chamber of the vacuum evaporation apparatus, and then the substrate was cooled down for approximately 30 minutes.

Next, the substrate provided with the anode 101 was fixed to a substrate holder provided in the vacuum evaporation apparatus such that the surface on which the anode 101 was formed faced downward, and N-[(3′,5′-ditertiarybutyl)-1,1′-biphenyl-4-yl]-N-(4-cyclohexylphenyl)-9,9-dimethyl-9H-fluoren-2-amine (abbreviation: mmtBuBichPAF) represented by Structural Formula (xi) above and an electron acceptor material (OCHD-001) were deposited by co-evaporation on the anode 101 to 10 nm by an evaporation method such that the weight ratio was 1:0.1 (=mmtBuBichPAF: OCHD-001).

Subsequently, mmtBuBichPAF was deposited to 50 nm by evaporation, and 4-(dibenzothiophen-4-yl)-4′-phenyl-4″-(9-phenyl-9H-carbazol-2-yl)triphenylamine (abbreviation: PCBBiPDBt-02) represented by (vii) was deposited to 50 nm by evaporation.

After that, PCBBiPDBt-02 and OCHD-001 were deposited by co-evaporation to 10 nm at a weight ratio of 1:0.1 (=PCBBiPDBt-02:OCHD-001).

Lastly, aluminum was deposited to 100 nm by evaporation; as a result, the device 3, which was a measurement device, was fabricated.

### (Fabrication Method of Device 4)

The device 4 was fabricated in a manner similar to that of the device 3 except that mmtBuBichPAF in the device 3 was replaced with N-(1,1′-biphenyl-2-yl)-N-(3,3″,5′,5″-tetra-t-butyl-1,1′:3′,1″-terphenyl-5-yl)-9,9-dimethyl-9H-fluoren-2-amine (abbreviation: mmtBumTPoFBi-02) represented by Structural Formula (i) above.

### (Fabrication Method of Comparative Device 4)

The comparative device 4 was fabricated in a manner similar to that of the device 3 except that mmtBuBichPAF in the device 3 was replaced with N-(1,1′-biphenyl-4-yl)-9,9-dimethyl-N-[4-(9-phenyl-9H-carbazol-3-yl)phenyl]-9H-fluoren-2-amine (abbreviation: PCBBiF) represented by Structural Formula (xii) above.

### (Fabrication Method of Comparative Device 5)

The comparative device 5 was fabricated in a manner similar to that of the comparative device 4 except that PCBBiPDBt-02 in the comparative device 4 was replaced with N,N-bis[4-(dibenzofuran-4-yl)phenyl]-4-amino-p-terphenyl (abbreviation: DBfBB1TP) represented by Structural Formula (xiii) above.

### (Fabrication Method of Comparative Device 6)

The comparative device 6 was fabricated in a manner similar to that of the device 3 except that PCBBiPDBt-02 in the device 3 was replaced with DBfBBITP.

### (Fabrication Method of Comparative Device 7)

The comparative device 7 was fabricated in a manner similar to that of the device 4 except that PCBBiPDBt-02 in the device 4 was replaced with DBfBBITP.

The element structures of the device 3, the device 4, and the comparative device 4 to the comparative device 7 are listed in the following table.

TABLE 3

| Film | Device | | Comparative device | | | |
|---|---|---|---|---|---|---|
| thickness | 3 | 4 | 4 | 5 | 6 | 7 |

| | | |
|---|---|---|
| 10 nm | PCBBiPDBt-02:OCHD-001 | DBfBB1TP:OCHD-001 |
| | (1:0.1) | (1:0.1) |
| 50 nm | PCBBiPDBt-02 | DBfBB1TP |

| | |
|---|---|
| 50 nm | *1 |
| 10 nm | *1:OCHD-001 |
| | (1:0.1) |

*1 Device 3, Comparative device 6: mmtBuBichPAF Device 4, Comparative device 7: mmtBumTPoFBi-02 Comparative devices 4, 5: PCBBiF

Note that among the organic compounds used for the devices, mmtBuBichPAF is an organic compound having a hole-transport property with a low refractive index: the ordinary refractive index in the entire blue light emission region (greater than or equal to 455 nm and less than or equal to 465 nm) is higher than or equal to 1.72 and lower than or equal to 1.73, which is within the range higher than or equal to 1.50 and lower than or equal to 1.75, and the ordinary refractive index at 633 nm is 1.65, which is within the range higher than or equal to 1.45 and lower than or equal to 1.70. Furthermore, mmtBumTPoFBi-02 is an organic compound having a hole-transport property with a low refractive index: the ordinary refractive index in the entire blue light emission region (greater than or equal to 455 nm and less than or equal to 465 nm) is higher than or equal to 1.69 and lower than or equal to 1.70, which is within the range higher than or equal to 1.50 and lower than or equal to 1.75, and the ordinary refractive index at 633 nm is 1.64, which is within the range higher than or equal to 1.45 and lower than or equal to 1.70.

Furthermore, among the organic compounds used for the devices, PCBBiPDBt-02 is a monoamine compound with a triarylamine structure having a group including a carbazole structure, a group including a dibenzofuran structure or a dibenzothiophene structure, and a group including an aromatic hydrocarbon structure having 6 to 18 carbon atoms. Note that DBfBB1TP does not include a carbazole structure and thus is an organic compound that does not have the above structure.

The light-emitting devices and the comparative light-emitting devices were sealed using a glass substrate in a glove box containing a nitrogen atmosphere so as not to be exposed to the air (a UV curable sealing material was applied to surround the elements, only the sealing material was irradiated with UV while the light-emitting devices were not irradiated with the UV, and heat treatment was performed at 80° C. under an atmospheric pressure for one hour), and then the initial characteristics of the light-emitting devices were measured.

Note that these devices are devices imitating the stacked-layer structure between the anode and the light-emitting layer of the light-emitting device, in which only holes flow. Measurement of such devices enables observation of hole injection and transport properties without being influenced by injection and transport of electrons, recombination in the light-emitting layer, and the like.

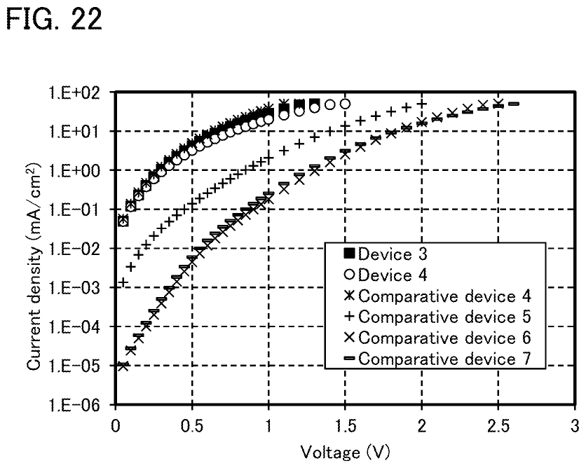

FIG. 22 shows current density-voltage characteristics of the device 3, the device 4, and the comparative device 4 to the comparative device 4.

FIG. 22 shows that, when the comparative device 6 and the comparative device 7 containing mmtBuBichPAF and mmtBumTPoFBi-02, which are low refractive index materials, are compared with the comparative device 5 containing no low refractive index material, the comparative devices 6 and 7 have low characteristics. This means that hole-injection properties and hole-transport properties of mmtBuBichPAF and mmtBumTPoFBi-02 are lower than those of PCBBiF. However, despite the use of low refractive index materials, the device 3 and the device 4 in which PCBBiPDBt-02 was formed in contact with mmtBuBichPAF and mmtBumTPoFBi-02 exhibited characteristics equivalent to those of the comparative device 4 containing no low refractive index material. It was found that when a monoamine compound with a triarylamine structure having a group including a carbazole structure, a group including a dibenzofuran structure or a dibenzothiophene structure, and a group including an aromatic hydrocarbon structure having 6 to 18 carbon atoms, such as PCBBiPDBt-02, was provided in contact with a low refractive index material as described above, the current density-voltage characteristics were dramatically improved.

### Example 3

In this example, a light-emitting device 5 and a light-emitting device 6, each of which is one embodiment of the present invention described in the embodiment, and a comparative light-emitting device 8 to a comparative light-emitting device 11 are described. Structural formulae of organic compounds used in this example are shown below.

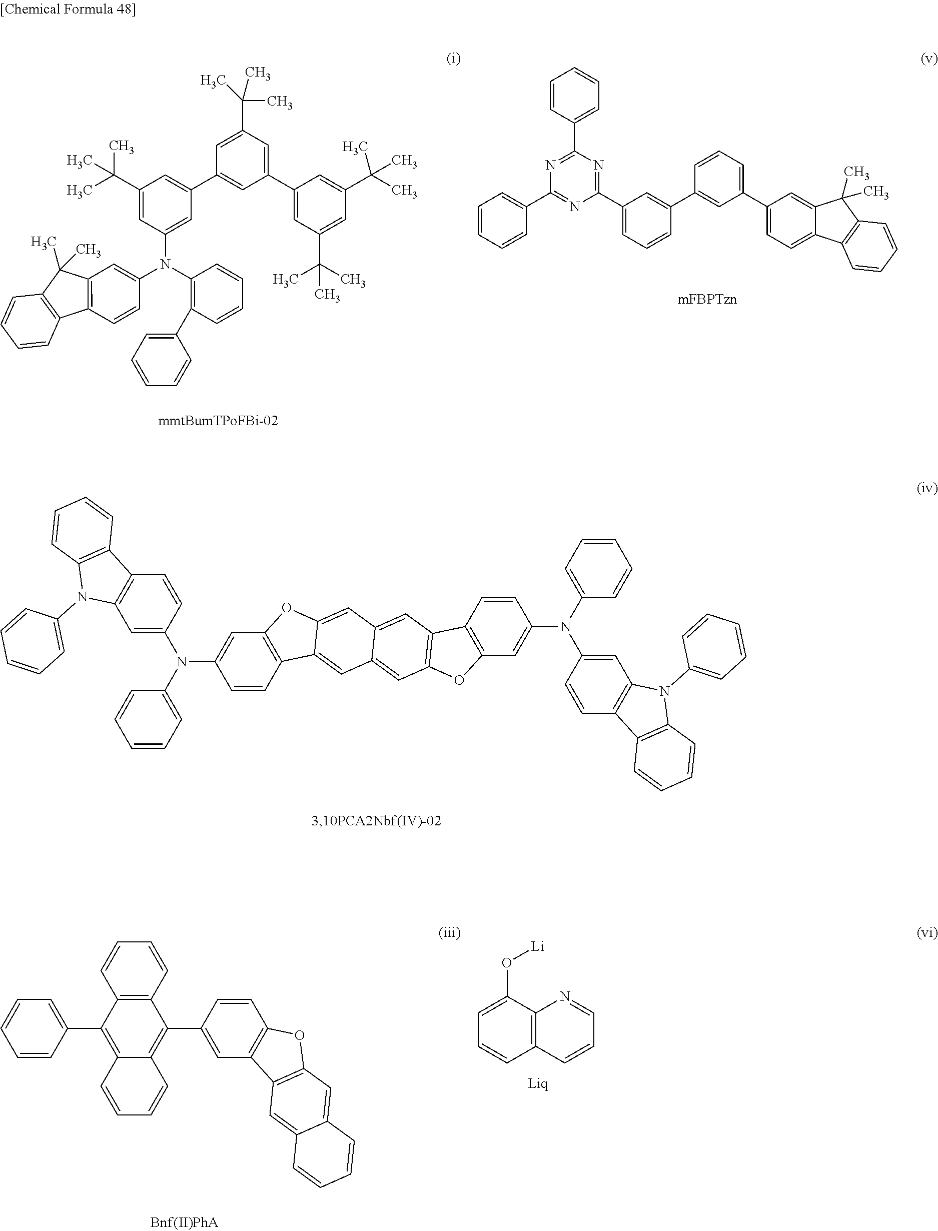

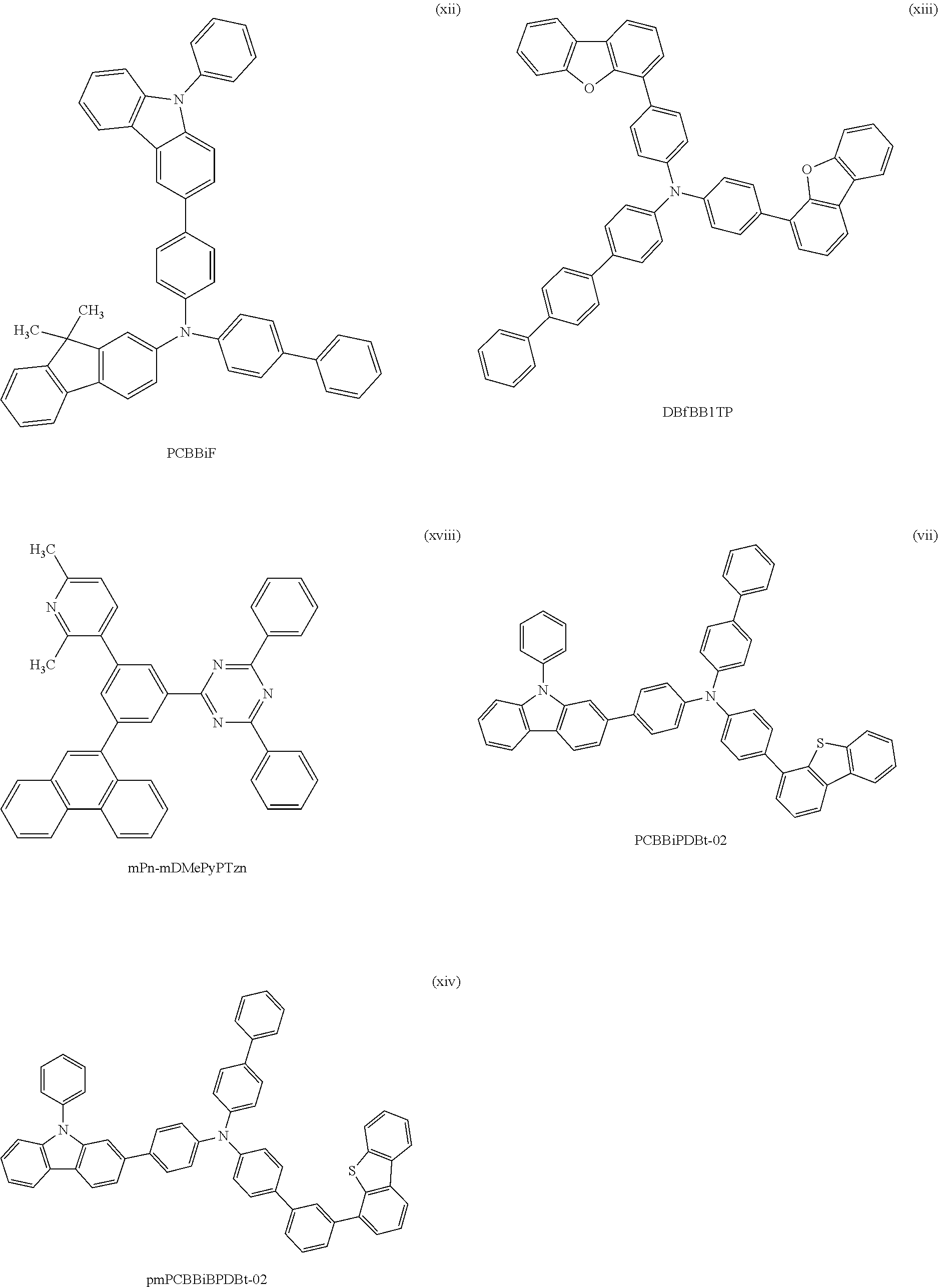

### (Fabrication Method of Light-Emitting Device 5)

First, as a transparent electrode, an indium tin oxide containing silicon oxide (ITSO) was deposited over a glass substrate to a thickness of 55 nm by a sputtering method, whereby the anode 101 was formed. The electrode area was set to 4 mm² (2 mm×2 mm).

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus where the pressure was reduced to approximately 104 Pa, vacuum baking was performed at 170° C. for 30 minutes in a heating chamber of the vacuum evaporation apparatus, and then the substrate was cooled down for approximately 30 minutes.

Next, the substrate provided with the anode 101 was fixed to a substrate holder provided in the vacuum evaporation apparatus such that the surface on which the anode 101 was formed faced downward, and N-(1,1′-biphenyl-2-yl)-N-(3,3″,5′,5″-tetra-t-butyl-1,1′: 3′,1″-terphenyl-5-yl)-9,9-dimethyl-9H-fluoren-2-amine (abbreviation: mmtBumTPoFBi-02) represented by Structural Formula (i) above and an electron acceptor material (OCHD-001) were deposited by co-evaporation on the anode 101 to 10 nm by an evaporation method such that the weight ratio was 1:0.1 (=mmtBumTPoFBi-02:OCHD-001), whereby the hole-injection layer 111 was formed.

Over the hole-injection layer 111, mmtBumTPoFBi-02 was deposited to 100 nm by evaporation, whereby the hole-transport layer 112 was formed.

Subsequently, over the hole-transport layer 112, 4-(dibenzothiophen-4-yl)-4′-phenyl-4″-(9-phenyl-9H-carbazol-2-yl)triphenylamine (abbreviation: PCBBiPDBt-02) represented by Structural Formula (vii) above was deposited to 10 nm by evaporation, whereby an electron-blocking layer was formed.

Then, 2-(10-phenyl-9-anthracenyl)-benzo[b]naphtho[2,3-d]furan (abbreviation: Bnf(II)PhA) represented by Structural Formula (iii) above and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iv) above were deposited to 25 nm by co-evaporation such that the weight ratio was 1:0.015 (=Bnf(II)PhA:3,10PCA2Nbf(IV)-02), whereby the light-emitting layer 113 was formed.

After that, 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (v) above was deposited to nm by evaporation, whereby a hole-blocking layer was formed. Furthermore, 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (xviii) above and 8-quinolinolato-lithium (abbreviation: Liq) represented by Structural Formula (vi) above were deposited to 15 nm by co-evaporation such that the weight ratio was 1:1 (=mPn-mDMePyPTzn:Liq), whereby the electron-transport layer 114 was formed.

After the electron-transport layer 114 was formed, Liq was deposited to 1 nm to form the electron-injection layer 115, and lastly aluminum was deposited to a thickness of 200 nm by evaporation to form the cathode 102, whereby the light-emitting device 5 was fabricated.

### (Fabrication Method of Light-Emitting Device 6)

The light-emitting device 6 was fabricated in a manner similar to that of the light-emitting device 5 except that PCBBiPDBt-02 in the electron-blocking layer of the light-emitting device 5 was replaced with 4-[3-(dibenzothiophen-4-yl)phenyl]-4′-phenyl-4″-(9-phenyl-9H-carbazol-2-yl)triphenylamine (abbreviation: pmPCBBiBPDBt-02) represented by Structural Formula (vii) above.

### (Fabrication Method of Comparative Light-Emitting Device 8)

The comparative light-emitting device 8 was fabricated in a manner similar to that of the light-emitting device 5 except that PCBBiPDBt-02 in the electron-blocking layer of the comparative device 5 was replaced with N,N-bis[4-(dibenzofuran-4-yl)phenyl]-4-amino-p-terphenyl (abbreviation: DBfBBITP) represented by Structural Formula (xiii) above.

### (Fabrication Method of Comparative Light-Emitting Device 9)

The comparative light-emitting device 9 was fabricated in a manner similar to that of the light-emitting device 5 except that mmtBumTPoFBi-02 in the hole-injection layer and the hole-transport layer of the light-emitting device 5 was replaced with N-(1,1′-biphenyl-4-yl)-9,9-dimethyl-N-[4-(9-phenyl-9H-carbazol-3-yl)phenyl]-9H-fluoren-2-amine (abbreviation: PCBBiF) represented by Structural Formula (xii) above.

### (Fabrication Method of Comparative Light-Emitting Device 10)

The comparative light-emitting device 10 was fabricated in a manner similar to that of the light-emitting device 6 except that mmtBumTPoFBi-02 in the hole-injection layer and the hole-transport layer of the light-emitting device 6 was replaced with PCBBiF.

### (Fabrication Method of Comparative Light-Emitting Device 11)

The comparative light-emitting device 11 was fabricated in a manner similar to that of the light-emitting device 6 except that mmtBumTPoFBi-02 in the hole-injection layer and the hole-transport layer of the comparative light-emitting device 8 was replaced with PCBBiF.

The element structures of the light-emitting device 5, the light-emitting device 6, and the comparative light-emitting device 8 to the comparative light-emitting device 11 are listed in the following tables.

TABLE 4

| | | | | Comparative |
|---|---|---|---|---|
| | | Light-emitting | Light-emitting | light-emitting |
| | | device 5 | device 6 | device 8 |
| Electron-injection layer | 1 nm | Liq | | |
| Electron-transport layer | 15 nm | mPn-mDMePyPTzn:Liq | | |
| | | (1:1) | | |
| Hole-blocking layer | 10 nm | mFBPTzn | | |
| Light-emitting layer | 25 nm | Bnf(II)PhA:3,10PCA2Nbf(IV)-02 | | |
| | | (1:0.015) | | |
| Electron-blocking layer | 10 nm | PCBBiPDBt-02 | pmPCBBiBPDBt-02 | DBfBB1TP |

| | | |
|---|---|---|
| Hole-transport layer | 100 nm | mmtBumTPoFBi-02 |
| Hole-injection layer | 10 nm | mmtBumTPoFBi-02:OCHD-001 |
| | | (1:0.1) |

TABLE 5

| | | Comparative | Comparative | Comparative |
|---|---|---|---|---|
| | | light-emitting | light-emitting | light-emitting |
| | | device 9 | device 10 | device 11 |
| Electron-injection layer | 1 nm | Liq | | |
| Electron-transport layer | 15 nm | mPn-mDMePyPTzn:Liq | | |
| | | (1:1) | | |
| Hole-blocking layer | 10 nm | mFBPTzn | | |
| Light-emitting layer | 25 nm | Bnf(II)PhA:3,10PCA2Nbf(IV)-02 | | |
| | | (1:0.015) | | |
| Electron-blocking layer | 10 nm | PCBBiPDBt-02 | pmPCBBiBPDBt-02 | DBfBB1TP |

| | | |
|---|---|---|
| Hole-transport layer | 100 nm | PCBBiF |
| Hole-injection layer | 10 nm | PCBBiF:OCHD-001 |
| | | (1:0.1) |

Note that mmtBumTPoFBi-02 is a hole-transport material with a low refractive index: the ordinary refractive index in the entire blue light emission region (greater than or equal to 455 nm and less than or equal to 465 nm) is higher than or equal to 1.69 and lower than or equal to 1.70, which is within the range higher than or equal to 1.50 and lower than or equal to 1.75, and the ordinary refractive index at 633 nm is 1.64, which is within the range higher than or equal to 1.45 and lower than or equal to 1.70. The ordinary refractive index of Bnf(II)PhA in the entire blue light emission region (greater than or equal to 455 nm and less than or equal to 465 nm) is higher than or equal to 1.89 and lower than or equal to 1.91, and the ordinary refractive index at 633 nm is 1.79. Since the concentration of 3,10PCA2Nbf(IV)-02 in the light-emitting layer 113 is low, the refractive index of the light-emitting layer 113 is equivalent to that of Bnf(II)PhA. Accordingly, the light-emitting device of one embodiment of the present invention is a light-emitting device in which the refractive index of the hole-transport layer 112 is lower than that of the light-emitting layer 113.

Furthermore, PCBBiPDBt-02 and pmPCBBiBPDBt-02 used in the electron-blocking layers of the light-emitting device 5 and the light-emitting device 6 are each a monoamine compound with a triarylamine structure having a group including a carbazole structure, a group including a dibenzofuran structure or a dibenzothiophene structure, and a group including an aromatic hydrocarbon structure having 6 to 18 carbon atoms.

The light-emitting devices and the comparative light-emitting devices were sealed using a glass substrate in a glove box containing a nitrogen atmosphere so as not to be exposed to the air (a UV curable sealing material was applied to surround the elements, only the sealing material was irradiated with UV while the light-emitting devices were not irradiated with the UV, and heat treatment was performed at 80° C. under an atmospheric pressure for one hour), and then the initial characteristics of the light-emitting devices were measured.

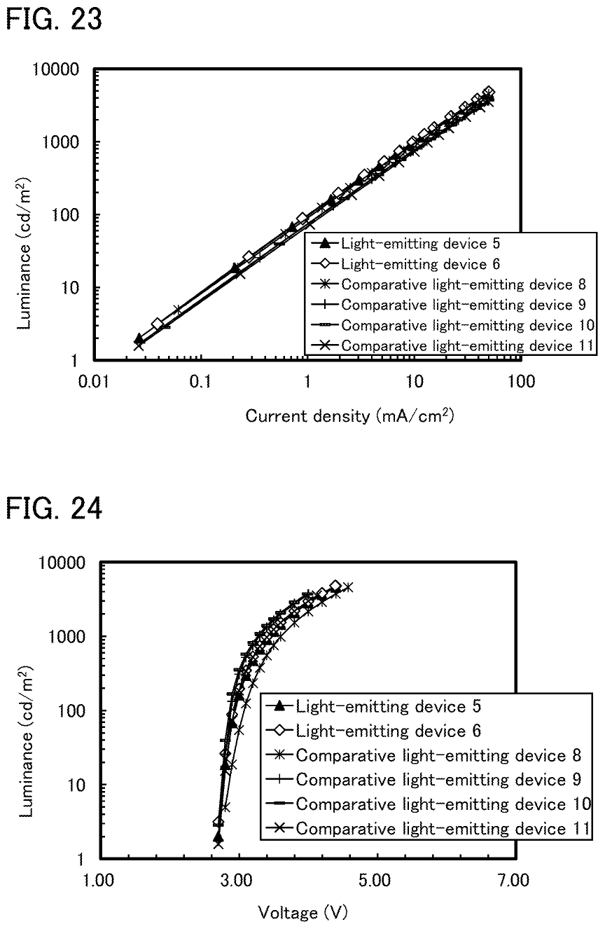

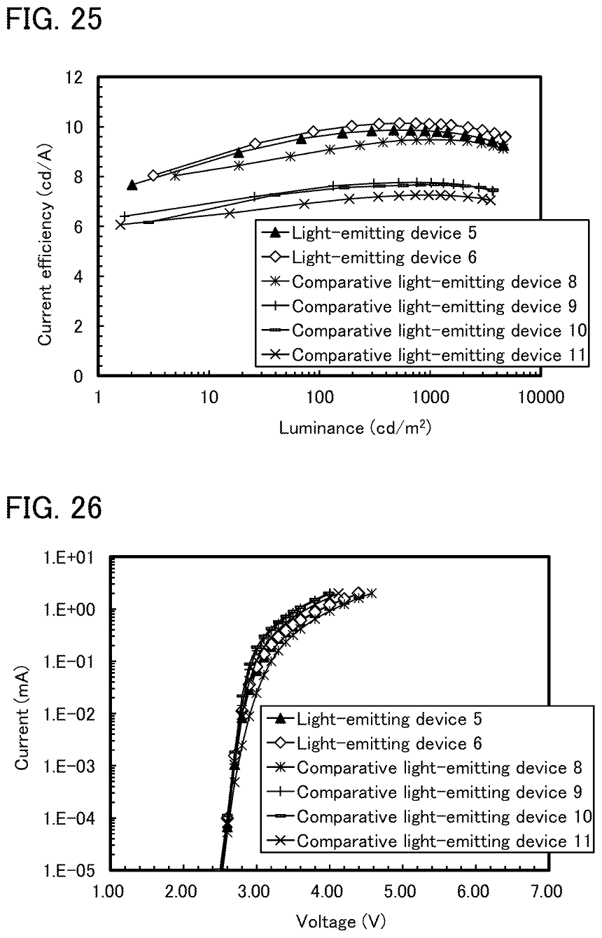

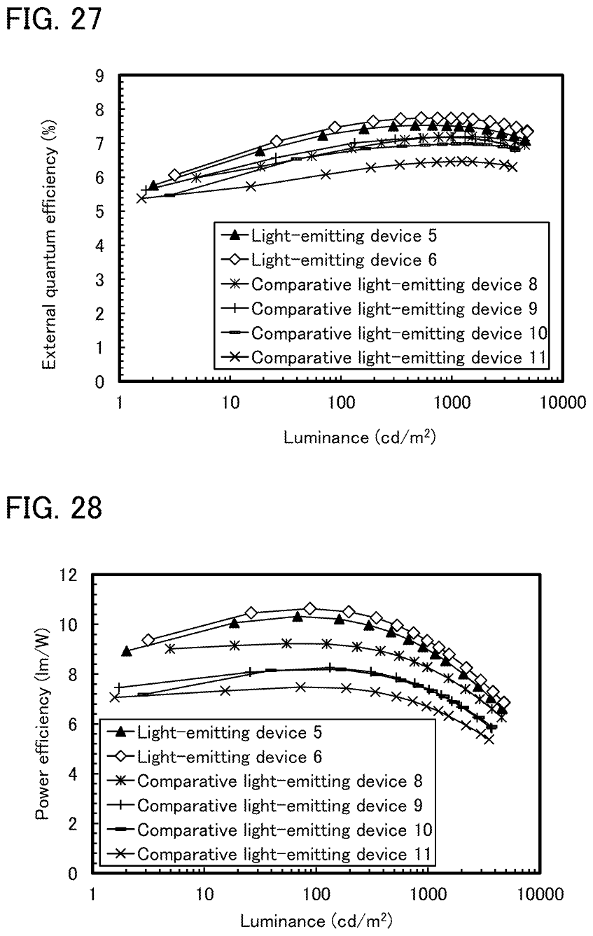

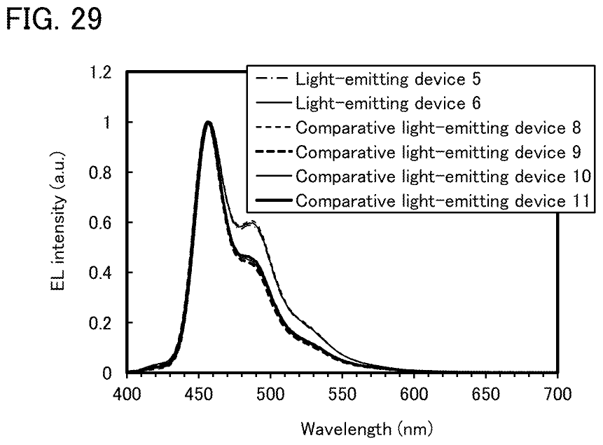

FIG. 23 shows the luminance-current density characteristics of the light-emitting device 5, the light-emitting device 6, and the comparative light-emitting device 8 to the comparative light-emitting device 11; FIG. 24 shows the luminance-voltage characteristics thereof; FIG. 25 shows the current efficiency-luminance characteristics thereof, FIG. 26 shows the current-voltage characteristics thereof, FIG. 27 shows the external quantum efficiency-luminance characteristics thereof, FIG. 28 shows the power efficiency-luminance characteristics thereof, and FIG. 29 shows the emission spectra thereof. Table 6 shows the main characteristics of the light-emitting device 5, the light-emitting device 6, and the comparative light-emitting device 8 to the comparative light-emitting device 11 at approximately 1000 cd/m². The luminance, CIE chromaticity, and emission spectra were measured at normal temperature with a spectroradiometer (SR-UL1R manufactured by TOPCON TECHNOHOUSE CORPORATION).

TABLE 6

| | | | Current | | Current | External |
|---|---|---|---|---|---|---|
| | Voltage | Current | density | Chromaticity | efficiency | quantum |
| | (V) | (mA) | (mA/cm²) | (x, y) | (cd/A) | efficiency (%) |
| Light-emitting device 5 | 3.4 | 0.37 | 9.1 | (0.141, 0.166) | 9.8 | 7.5 |
| Light-emitting device 6 | 3.4 | 0.39 | 9.7 | (0.140, 0.166) | 10.1 | 7.7 |
| Comparative light-emitting | 3.6 | 0.42 | 10.4 | (0.139, 0.168) | 9.5 | 7.2 |
| device 8 | | | | | | |
| Comparative light-emitting | 3.3 | 0.53 | 13.2 | (0.143, 0.126) | 7.8 | 7.2 |
| device 9 | | | | | | |
| Comparative light-emitting | 3.3 | 0.57 | 14.2 | (0.143, 0.129) | 7.7 | 7.0 |
| device 10 | | | | | | |
| Comparative light-emitting | 3.3 | 0.40 | 10.1 | (0.142, 0.133) | 7.3 | 6.4 |
| device 11 | | | | | | |

It was found from FIG. 23 to FIG. 29 and Table 6 that the light-emitting device 5 and the light-emitting device 6, each of which was one embodiment of the present invention, were light-emitting elements with favorable power efficiency because they had favorable external quantum efficiency and a decrease in driving voltage was inhibited owing to PCBBiPDBt-02 and pmPCBBiBPDBt-02 provided in contact with mmtBumTPoFBi-02, which was a low refractive index material. Note that pmPCBBiBPDBt-02 in which a dibenzothiophenyl group is bonded to nitrogen of amine through a meta-substituted phenylene group is further preferable because of its significant effect of improving efficiency.

As described above, a light-emitting device in which a layer containing a monoamine compound having a specific structure is formed in contact with a low refractive index layer can be a light-emitting device with favorable characteristics. Specifically, the light-emitting device can have a favorable lifetime. Alternatively, the light-emitting device can have favorable emission efficiency. Alternatively, the light-emitting device can have a low driving voltage.

### Example 4

In this example, a light-emitting device 7, which is one embodiment of the present invention described in the embodiment, is described. Structural formulae of organic compounds used in this example are shown below.

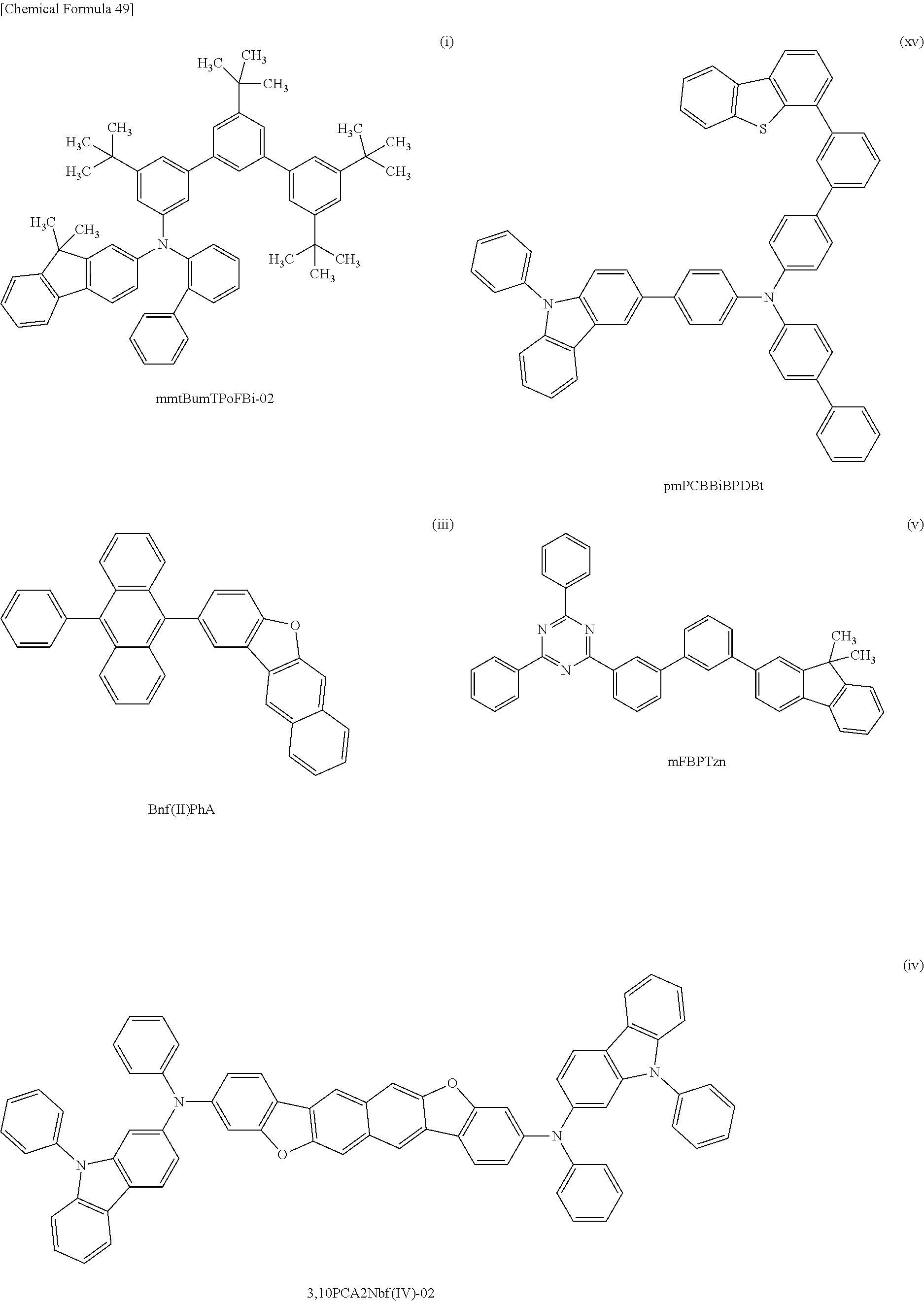 [SMILES: CC(C)(C)c1cc(-c2cc(N(c3ccc4c(c3)C(C)(C)c3ccccc3-4)c3ccccc3-c3ccccc3)cc(C(C)(C)C)c2)cc(-c2cc(C(C)(C)C)cc(C(C)(C)C)c2)c1 ; c1ccc(-c2ccc(N(c3ccc(-c4cccc(-c5cccc6c5sc5ccccc56)c4)cc3)c3ccc(-c4ccc5c(c4)c4ccccc4n5-c4ccccc4)cc3)cc2)cc1 ; c1ccc(-c2c3ccccc3c(-c3ccc4oc5cc6ccccc6cc5c4c3)c3ccccc23)cc1 ; CC1(C)c2ccccc2-c2ccc(-c3cccc(-c4cccc(-c5nc(-c6ccccc6)nc(-c6ccccc6)n5)c4)c3)cc21 ; c1ccc(N(c2ccc3c(c2)oc2cc4cc5c(cc4cc23)oc2cc(N(c3ccccc3)c3ccc4c6ccccc6n(-c6ccccc6)c4c3)ccc25)c2ccc3c4ccccc4n(-c4ccccc4)c3c2)cc1]

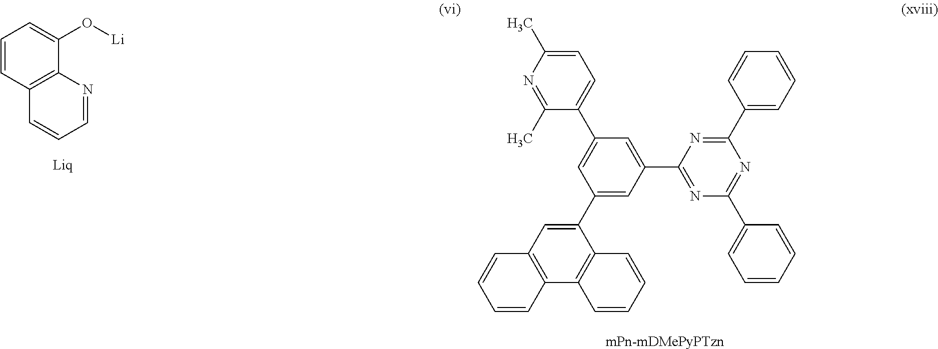

### (Fabrication Method of Light-Emitting Device 7)

First, as a transparent electrode, an indium tin oxide containing silicon oxide (ITSO) was deposited over a glass substrate to a thickness of 55 nm by a sputtering method, whereby the anode 101 was formed. The electrode area was set to 4 mm² (2 mm×2 mm).

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus where the pressure was reduced to approximately 104 Pa, vacuum baking was performed at 170° C. for 30 minutes in a heating chamber of the vacuum evaporation apparatus, and then the substrate was cooled down for approximately 30 minutes.

Next, the substrate provided with the anode 101 was fixed to a substrate holder provided in the vacuum evaporation apparatus such that the surface on which the anode 101 was formed faced downward, and N-(1,1′-biphenyl-2-yl)-N-(3,3″,5′,5″-tetra-t-butyl-1,1′:3′,1″-terphenyl-5-yl)-9,9-dimethyl-9H-fluoren-2-amine (abbreviation: mmtBumTPoFBi-02) represented by Structural Formula (i) above and an electron acceptor material (OCHD-001) were deposited by co-evaporation on the anode 101 to 10 nm by an evaporation method such that the weight ratio was 1:0.1 (=mmtBumTPoFBi-02:OCHD-001), whereby the hole-injection layer 111 was formed.

Over the hole-injection layer 111, mmtBumTPoFBi-02 was deposited to 100 nm by evaporation, whereby the hole-transport layer 112 was formed.

Subsequently, over the hole-transport layer 112, 4-[3-(dibenzothiophen-4-yl)phenyl]-4′-phenyl-4″-(9-phenyl-9H-carbazol-3-yl)triphenylamine (abbreviation: pmPCBBiBPDBt) represented by Structural Formula (xv) above was deposited to a thickness of 10 nm by evaporation, whereby an electron-blocking layer was formed.

Then, 2-(10-phenyl-9-anthracenyl)-benzo[b]naphtho[2,3-d]furan (abbreviation: Bnf(II)PhA) represented by Structural Formula (iii) above and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iv) above were deposited to 25 nm by co-evaporation such that the weight ratio was 1:0.015 (=Bnf(II)PhA:3,10PCA2Nbf(IV)-02), whereby the light-emitting layer 113 was formed.

After that, 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (v) above was deposited to nm by evaporation, whereby a hole-blocking layer was formed. Furthermore, 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (xviii) above and 8-quinolinolato-lithium (abbreviation: Liq) represented by Structural Formula (vi) above were deposited to 15 nm by co-evaporation such that the weight ratio was 1:1 (=mPn-mDMePyPTzn:Liq), whereby the electron-transport layer 114 was formed.

After the electron-transport layer 114 was formed, Liq was deposited to 1 nm to form the electron-injection layer 115, and lastly aluminum was deposited to a thickness of 200 nm by evaporation to form the cathode 102, whereby the light-emitting device 7 was fabricated.

The element structure of the light-emitting device 7 is listed in the following table.

TABLE 7

| | | Light-emitting device 7 |
|---|---|---|
| Electron-injection layer | 1 nm | Liq |
| Electron-transport layer | 15 nm | mPn-mDMePyPTzn:Liq |
| | | (1:1) |
| Hole-blocking layer | 10 nm | mFBPTzn |
| Light-emitting layer | 25 nm | Bnf(II)PhA:3,10PCA2Nbf(IV)-02 |
| | | (1:0.015) |
| Electron-blocking layer | 10 nm | pmPCBBiBPDBt |
| Hole-transport layer | 100 nm | mmtBumTPoFBi-02 |
| Hole-injection layer | 10 nm | mmtBumTPoFBi-02:OCHD-001 |
| | | (1:0.1) |

Note that mmtBumTPoFBi-02 is a hole-transport material with a low refractive index: the ordinary refractive index in the entire blue light emission region (greater than or equal to 455 nm and less than or equal to 465 nm) is higher than or equal to 1.69 and lower than or equal to 1.70, which is within the range higher than or equal to 1.50 and lower than or equal to 1.75, and the ordinary refractive index at 633 nm is 1.64, which is within the range higher than or equal to 1.45 and lower than or equal to 1.70. The ordinary refractive index of Bnf(II)PhA in the entire blue light emission region (greater than or equal to 455 nm and less than or equal to 465 nm) is higher than or equal to 1.89 and lower than or equal to 1.91, and the ordinary refractive index at 633 nm is 1.79. Since the concentration of 3,10PCA2Nbf(IV)-02 in the light-emitting layer 113 is low, the refractive index of the light-emitting layer 113 is equivalent to that of Bnf(II)PhA. Accordingly, the light-emitting device of one embodiment of the present invention is a light-emitting device in which the refractive index of the hole-transport layer 112 is lower than that of the light-emitting layer 113.

Furthermore, pmPCBBiBPDBt used in the electron-blocking layer of the light-emitting device 7 is a monoamine compound with a triarylamine structure having a group including a carbazole structure, a group including a dibenzofuran structure or a dibenzothiophene structure, and a group including an aromatic hydrocarbon structure having 6 to 18 carbon atoms.

The light-emitting device was sealed using a glass substrate in a glove box containing a nitrogen atmosphere so as not to be exposed to the air (a UV curable sealing material was applied to surround the element, only the sealing material was irradiated with UV while the light-emitting device was not irradiated with the UV, and heat treatment was performed at 80° C. under an atmospheric pressure for one hour), and then the initial characteristics of the light-emitting device were measured.

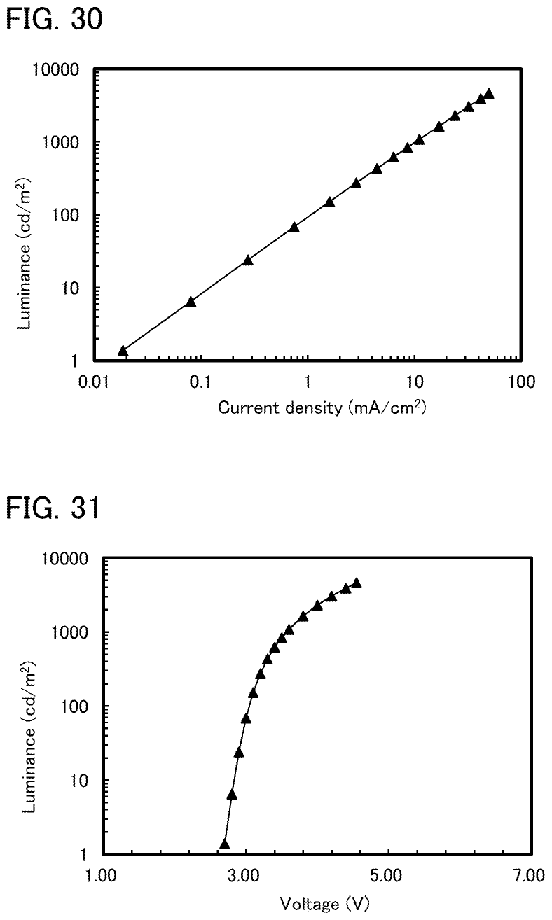

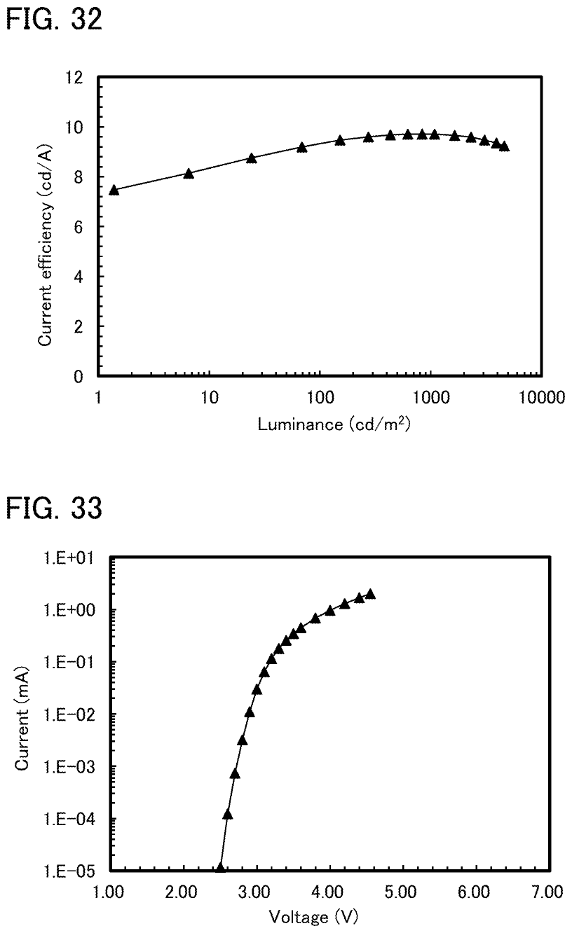

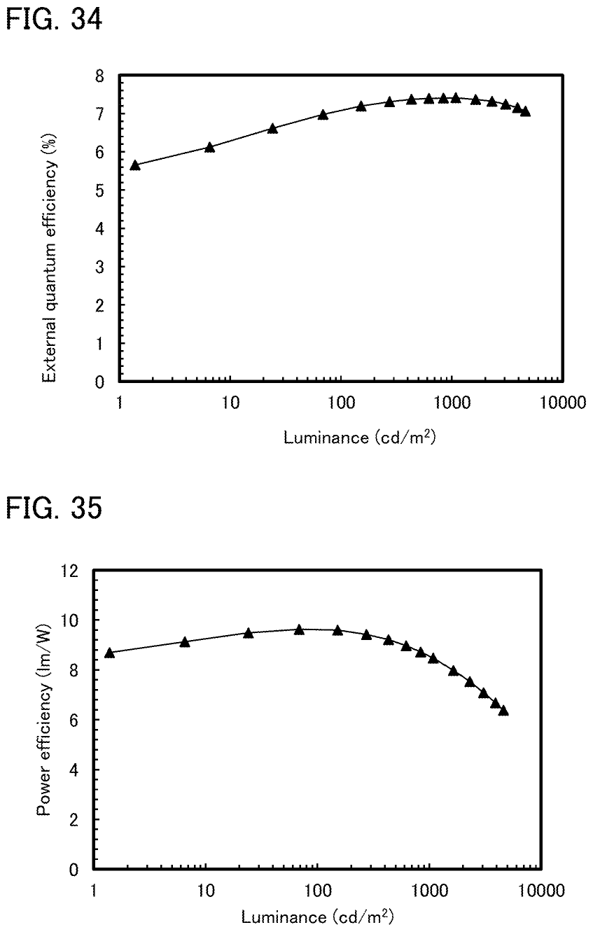

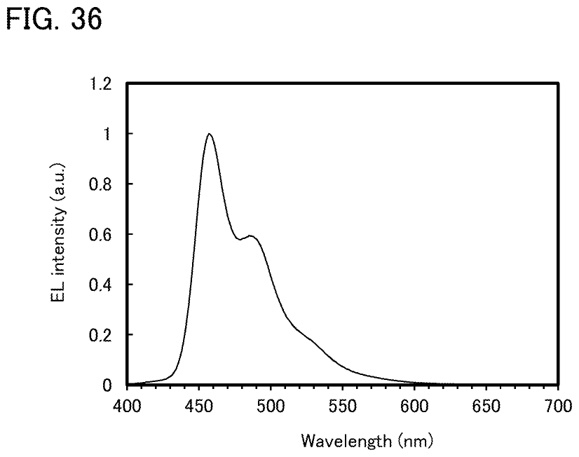

FIG. 30 shows the luminance-current density characteristics of the light-emitting device 7; FIG. 31 shows the luminance-voltage characteristics thereof, FIG. 32 shows the current efficiency-luminance characteristics thereof, FIG. 33 shows the current-voltage characteristics thereof, FIG. 34 shows the external quantum efficiency-luminance characteristics thereof, FIG. shows the power efficiency-luminance characteristics thereof, and FIG. 36 shows the emission spectrum thereof. Table 3 shows the main characteristics of the light-emitting device 7 at approximately 1000 cd/m². The luminance, CIE chromaticity, and emission spectrum was measured at normal temperature with a spectroradiometer (SR-UL1R manufactured by TOPCON TECHNOHOUSE CORPORATION).

TABLE 8

| | | | Current | | Current | External |
|---|---|---|---|---|---|---|
| | Voltage | Current | density | Chromaticity | efficiency | quantum |
| | (V) | (mA) | (mA/cm²) | (x, y) | (cd/A) | efficiency (%) |
| Light-emitting device 7 | 3.6 | 0.45 | 11.2 | (0.140, 0.166) | 9.7 | 7.4 |

It was found from FIG. 30 to FIG. 36 and Table 8 that the light-emitting device 7, which was one embodiment of the present invention, was a light-emitting element with favorable power efficiency because it had favorable external quantum efficiency and a decrease in driving voltage was inhibited owing to pmPCBBiBPDBt provided in contact with mmtBumTPoFBi-02, which was a low refractive index material.

As described above, a light-emitting device in which a layer containing a monoamine compound having a specific structure is formed in contact with a low refractive index layer can be a light-emitting device with favorable characteristics. Specifically, the light-emitting device can have a favorable lifetime. Alternatively, the light-emitting device can have favorable emission efficiency. Alternatively, the light-emitting device can have a low driving voltage.

### Example 5

In this example, a light-emitting device 8, which is one embodiment of the present invention described in the embodiment, and a comparative light-emitting device 12 are described. Structural formulae of organic compounds used in this example are shown below.

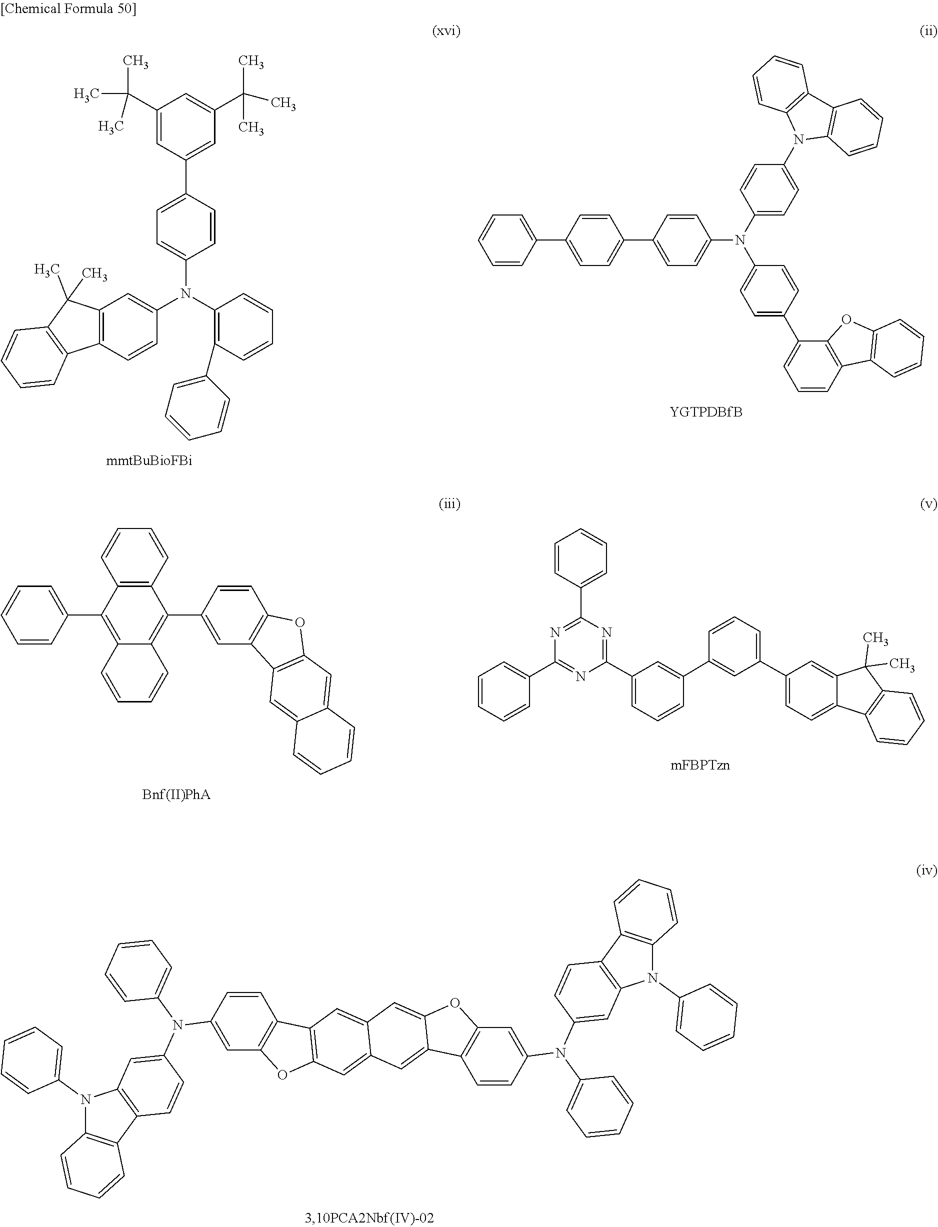 [SMILES: CC(C)(C)c1cc(-c2ccc(N(c3ccc4c(c3)C(C)(C)c3ccccc3-4)c3ccccc3-c3ccccc3)cc2)cc(C(C)(C)C)c1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2c3ccccc3c(-c3ccc4oc5cc6ccccc6cc5c4c3)c3ccccc23)cc1 ; CC1(C)c2ccccc2-c2ccc(-c3cccc(-c4cccc(-c5nc(-c6ccccc6)nc(-c6ccccc6)n5)c4)c3)cc21 ; c1ccc(N(c2ccc3c(c2)oc2cc4cc5c(cc4cc23)oc2cc(N(c3ccccc3)c3ccc4c6ccccc6n(-c6ccccc6)c4c3)ccc25)c2ccc3c4ccccc4n(-c4ccccc4)c3c2)cc1]

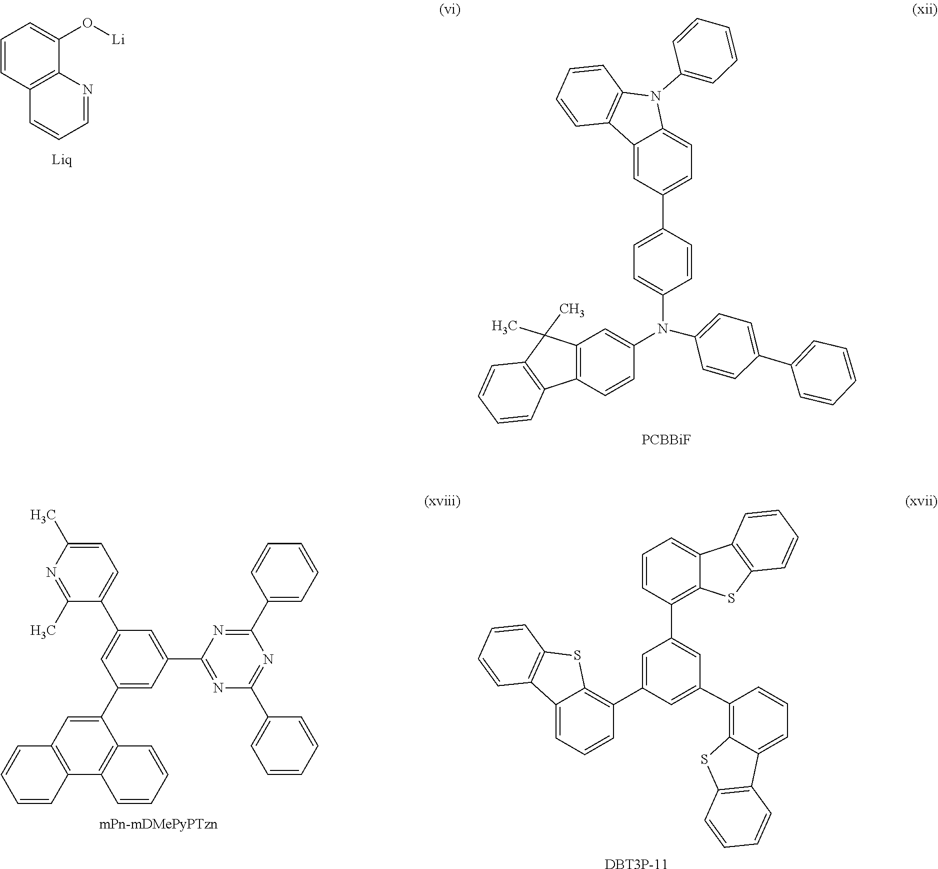

### (Fabrication Method of Light-Emitting Device 8)

First, silver (Ag) was formed over a glass substrate to a thickness of 100 nm by a sputtering method as a reflective electrode, and then an indium tin oxide containing silicon oxide (ITSO) was deposited to a thickness of 10 nm by a sputtering method as a transparent electrode, whereby the anode 101 was formed. The electrode area was set to 4 mm² (2 mm×2 mm).

Next, in pretreatment for forming the light-emitting device over the substrate, a surface of the substrate was washed with water and baked at 200° C. for 1 hour, and then UV ozone treatment was performed for 370 seconds.

After that, the substrate was transferred into a vacuum evaporation apparatus where the pressure was reduced to approximately 104 Pa, vacuum baking was performed at 170° C. for 30 minutes in a heating chamber of the vacuum evaporation apparatus, and then the substrate was cooled down for approximately 30 minutes.

Next, the substrate provided with the anode 101 was fixed to a substrate holder provided in the vacuum evaporation apparatus such that the surface on which the anode 101 was formed faced downward, and N-3′,5′-ditertiarybutyl-1,1′-biphenyl-4-yl-N-1,1′-biphenyl-2-yl-9,9-dimethyl-9H-fluoren-2-amine (abbreviation: mmtBuBioFBi) represented by Structural Formula (xvi) above and an electron acceptor material (OCHD-001) were deposited by co-evaporation on the anode 101 to 10 nm by an evaporation method such that the weight ratio was 1:0.1 (=mmtBumTPoFBi-02:OCHD-001), whereby the hole-injection layer 111 was formed.

Over the hole-injection layer 111, mmtBuBioFBi was deposited to 120 nm by evaporation, whereby the hole-transport layer 112 was formed.

Subsequently, over the hole-transport layer 112, N-[4-(9Hcarbazol-9-yl)phenyl]-N-[4-(4-dibenzofuranyl)phenyl]-[1,1′:4′,1″-terphenyl]-4-amine(abbreviation: YGTPDBfB) represented by Structural Formula (ii) above was deposited to a thickness of 10 nm by evaporation, whereby an electron-blocking layer was formed.

Then, 2-(10-phenyl-9-anthracenyl)-benzo[b]naphtho[2,3-d]furan (abbreviation: Bnf(II)PhA) represented by Structural Formula (iii) above and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iv) above were deposited to 25 nm by co-evaporation such that the weight ratio was 1:0.015 (=Bnf(II)PhA:3,10PCA2Nbf(IV)-02), whereby the light-emitting layer 113 was formed.

After that, 2-[3′-(9,9-dimethyl-9H-fluoren-2-yl)-1,1′-biphenyl-3-yl]-4,6-diphenyl-1,3,5-triazine (abbreviation: mFBPTzn) represented by Structural Formula (v) above was deposited to nm by evaporation, whereby a hole-blocking layer was formed. Furthermore, 2-[3-(2,6-dimethyl-3-pyridinyl)-5-(9-phenanthrenyl)phenyl]-4,6-diphenyl-1,3,5-triazine (abbreviation: mPn-mDMePyPTzn) represented by Structural Formula (xviii) above and 8-quinolinolato-lithium (abbreviation: Liq) represented by Structural Formula (vi) above were deposited to 20 nm by co-evaporation such that the weight ratio was 1:1 (=mPn-mDMePyPTzn:Liq), whereby the electron-transport layer 114 was formed.

After the electron-transport layer 114 was formed, lithium fluoride (LiF) was deposited to 1 nm to form the electron-injection layer 115, and lastly silver (Ag) and magnesium (Mg) were deposited to a thickness of 15 nm by co-evaporation such that the volume ratio was 1:0.1 to form the cathode 102, whereby the light-emitting device 8 was fabricated. Note that the cathode 102 is a transflective electrode having a function of reflecting light and a function of transmitting light; thus, the light-emitting device of this example is a top-emission element in which light is extracted through the cathode 102. Furthermore, over the cathode 102, 4,4′,4″-(benzene-1,3,5-triyl)tri(dibenzothiophene) (abbreviation: DBT3P-II) represented by Structural Formula (xvii) above was deposited to 70 nm by evaporation to improve outcoupling efficiency.

### (Fabrication Method of Comparative Light-Emitting Device 12)

The comparative light-emitting device 12 was fabricated in a manner similar to that of the light-emitting device 8 except that mmtBuBioFBi in the hole-injection layer and the hole-transport layer of the light-emitting device 8 was replaced with N-(1,1′-biphenyl-4-yl)-9,9-dimethyl-N-[4-(9-phenyl-9H-carbazol-3-yl)phenyl]-9H-fluoren-2-amine (abbreviation: PCBBiF) represented by Structural Formula (xii) above and the thickness of the hole-transport layer was set to 100 nm.

The element structures of the light-emitting device 8 and the comparative light-emitting device 12 are listed in the following table.

TABLE 9

| | | Light-emitting | Comparative light- |
|---|---|---|---|
| | | device 8 | emitting device 12 |
| Electron-injection layer | 1 nm | LiF | |
| Electron-transport layer | 20 nm | mPn-mDMePyPTzn:Liq | |
| | | (1:1) | |
| Hole-blocking layer | 10 nm | mFBPTzn | |
| Light-emitting layer | 25 nm | Bnf(II)PhA:3,10PCA2Nbf(IV)-02 | |
| | | (1:0.015) | |
| Electron-blocking layer | 10 nm | YGTPDBfB | |
| Hole-transport layer | *2 | mmtBuBioFBi | PCBBiF |
| Hole-injection layer | 10 nm | mmtBuBioFBi:OCHD-001 | PCBBiF:OCHD-001 |
| | | (1:0.1) | (1:0.1) |

*2 Light-emitting device 8: 120 nm, Comparative light-emitting device 12: 100 nm

Note that mmtBuBioFBi is a hole-transport material with a low refractive index: the ordinary refractive index in the entire blue light emission region (greater than or equal to 455 nm and less than or equal to 465 nm) is higher than or equal to 1.73 and lower than or equal to 1.74, which is within the range higher than or equal to 1.50 and lower than or equal to 1.75, and the ordinary refractive index at 633 nm is 1.66, which is within the range higher than or equal to 1.45 and lower than or equal to 1.70. The ordinary refractive index of Bnf(H)PhA in the entire blue light emission region (greater than or equal to 455 nm and less than or equal to 465 nm) is higher than or equal to 1.89 and lower than or equal to 1.91, and the ordinary refractive index at 633 nm is 1.79. Since the concentration of 3,10PCA2Nbf(IV)-02 in the light-emitting layer 113 is low, the refractive index of the light-emitting layer 113 is equivalent to that of Bnf(H)PhA. Accordingly, the light-emitting device of one embodiment of the present invention is a light-emitting device in which the refractive index of the hole-transport layer 112 is lower than that of the light-emitting layer 113.

Furthermore, YGTPDBfB used in the electron-blocking layer of the light-emitting device 8 is a monoamine compound with a triarylamine structure having a group including a carbazole structure, a group including a dibenzofuran structure or a dibenzothiophene structure, and a group including an aromatic hydrocarbon structure having 6 to 18 carbon atoms.

The light-emitting devices were sealed using a glass substrate in a glove box containing a nitrogen atmosphere so as not to be exposed to the air (a UV curable sealing material was applied to surround the elements, only the sealing material was irradiated with UV while the light-emitting devices were not irradiated with the UV, and heat treatment was performed at 80° C. under an atmospheric pressure for one hour), and then the initial characteristics of the light-emitting devices were measured.

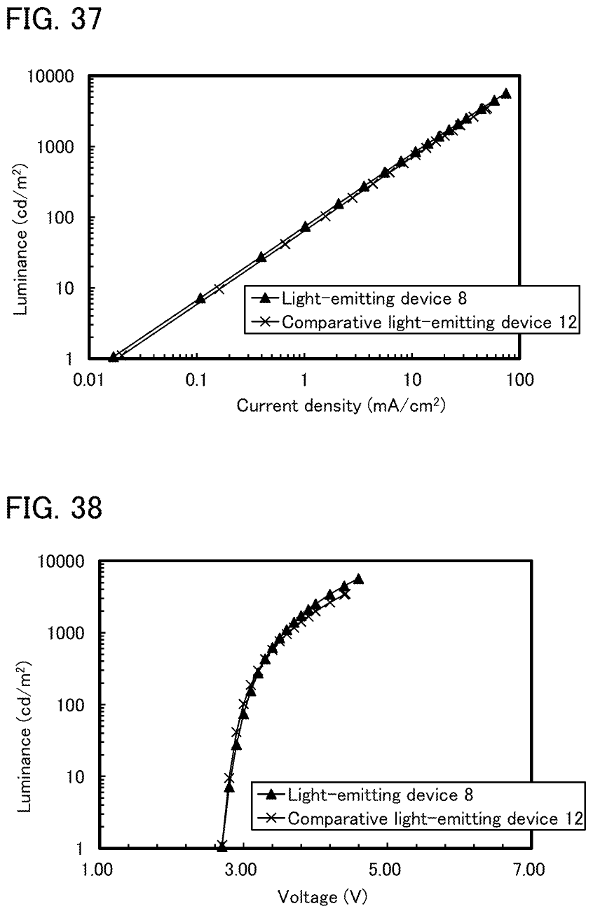

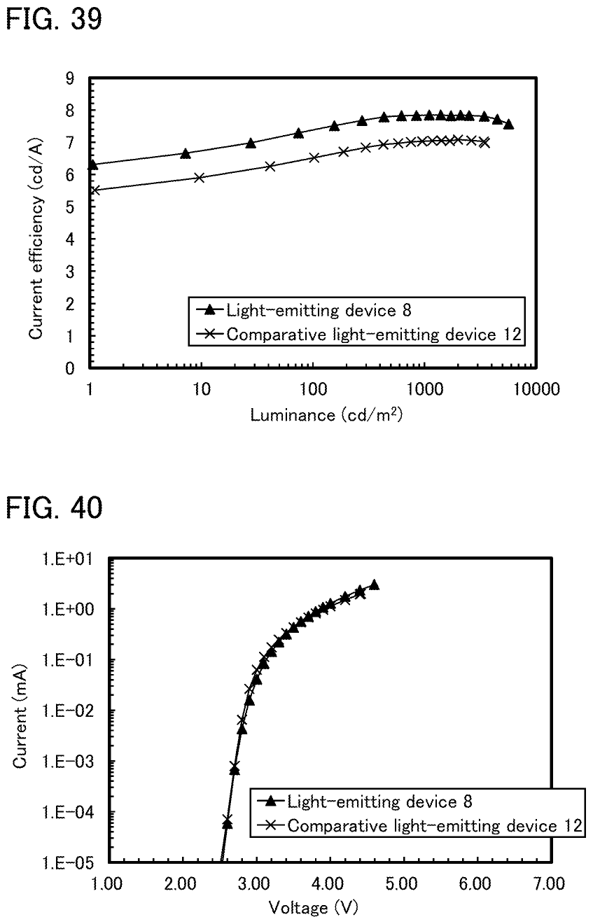

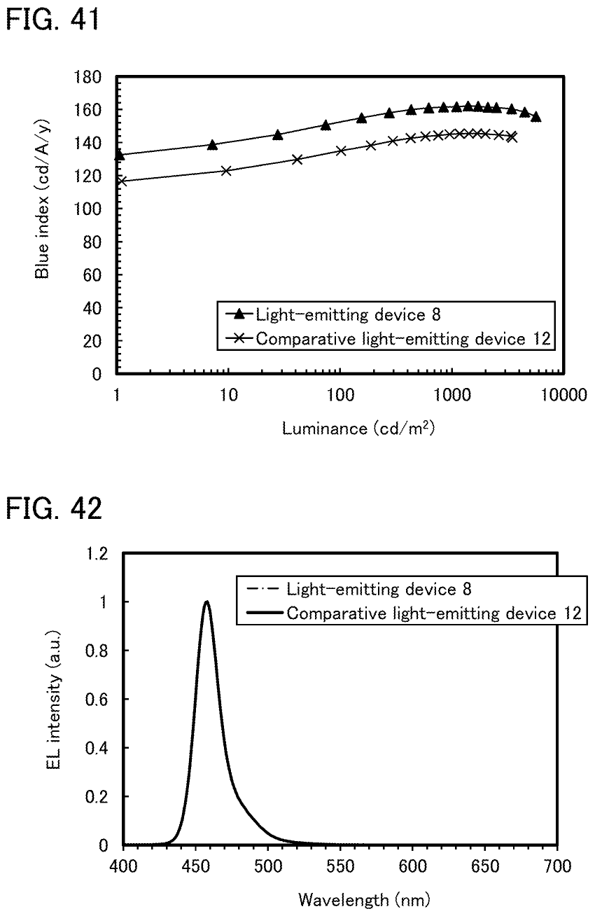

FIG. 37 shows the luminance-current density characteristics of the light-emitting device 8 and the comparative light-emitting device 12; FIG. 38 shows the luminance-voltage characteristics thereof, FIG. 39 shows the current efficiency-luminance characteristics thereof, FIG. 40 shows the current-voltage characteristics thereof, FIG. 41 shows the blue index-luminance characteristics thereof, and FIG. 42 shows the emission spectra thereof. Table 10 shows the main characteristics of the light-emitting device 8 and the comparative light-emitting device 12 at approximately 1000 cd/m². The luminance, CIE chromaticity, and emission spectra were measured at normal temperature with a spectroradiometer (SR-UL1R manufactured by TOPCON TECHNOHOUSE CORPORATION).

TABLE 10

| | | | Current | | Current | |
|---|---|---|---|---|---|---|
| | Voltage | Current | density | Chromaticity | efficiency | BI |
| | (V) | (mA) | (mA/cm²) | (x, y) | (cd/A) | (cd/A/y) |
| Light-emitting | 3.6 | 0.56 | 14.1 | (0.141, 0.049) | 7.8 | 162 |
| device 8 | | | | | | |
| Comparative | 3.6 | 0.54 | 13.6 | (0.140, 0.049) | 7.0 | 145 |
| light-emitting | | | | | | |
| device 12 | | | | | | |

It was found from FIG. 37 to FIG. 42 and Table 10 that the light-emitting device 7, which was one embodiment of the present invention, was a light-emitting element with favorable emission efficiency because YGTPDBfB therein was provided in contact with mmtBuBioFBi, which was a low refractive index material.

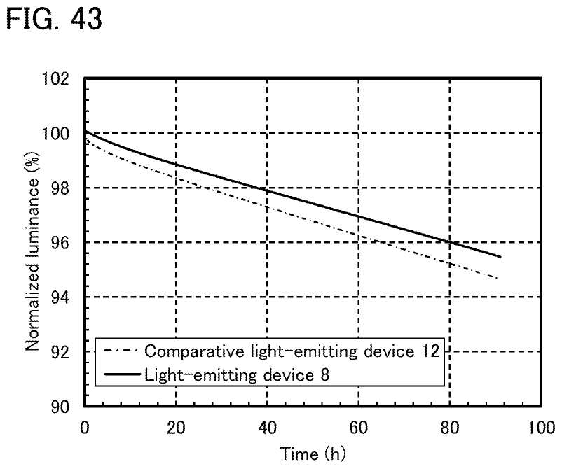

FIG. 43 is a graph showing a change in luminance over driving time at a current density of 50 mA/cm². As shown in FIG. 43, the light-emitting device 8 was found to be a light-emitting device with a favorable lifetime.

As described above, a light-emitting device in which a layer containing a monoamine compound having a specific structure is formed in contact with a low refractive index layer can be a light-emitting device with favorable characteristics. Specifically, the light-emitting device can have favorable emission efficiency. Alternatively, the light-emitting device can have a low driving voltage. Alternatively, the light-emitting device can have a favorable lifetime.

### Example 6

### Synthesis Example 1

In this example, a synthesis method of 4-(dibenzothiophen-4-yl)-4′-phenyl-4″-(9-phenyl-9H-carbazol-2-yl)triphenylamine (abbreviation: PCBBiPDBt-02), which is the monoamine compound of one embodiment of the present invention represented by General Formula (G1) described in Embodiment 1, will be described. The structure of PCBBiPDBt-02 is shown below.

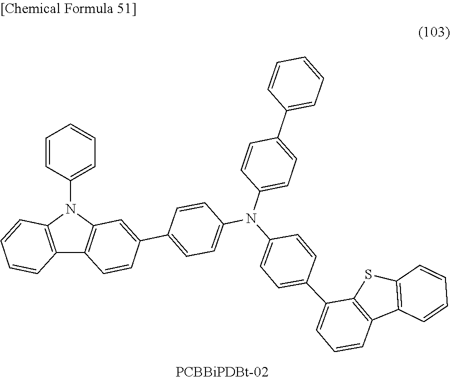

Into a 200-mL three-neck flask were put 1.8 g (3.7 mmol) of N-(4-biphenyl)-N-[4-(9-phenyl-9H-carbazol-2-yl)phenyl]amine, 1.1 g (3.4 mmol) of 4-(4-bromophenyl)dibenzothiophene, and 0.97 g (10 mmol) of sodium tert-butoxide. To this mixture was added 20 mL of toluene and 0.2 mL of a 10% hexane solution of tri(tert-butyl)phosphine, and this mixture was degassed by being stirred while the pressure was being reduced. To this mixture was added 19 mg (34 μmol) of bis(dibenzylideneacetone)palladium(0), and the mixture was heated and stirred at 110° C. for 9 hours under a nitrogen stream.

After the stirring, toluene was added to this mixture, and the resulting mixture was suction-filtered through Florisil, Celite, and alumina. The obtained filtrate was concentrated to give a solid. The synthesis scheme of this synthesis example is shown below.

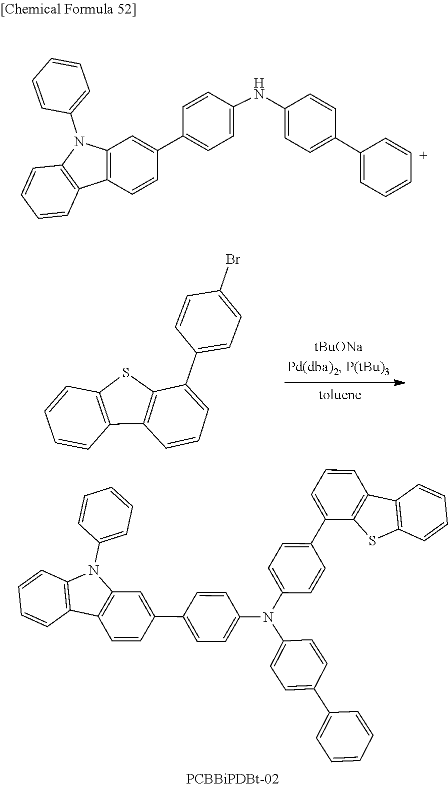 [SMILES: [C+]1=CC(c2ccc(Nc3ccc(-c4ccc5c6ccccc6n(-c6ccccc6)c5c4)cc3)cc2)=CC=C1 ; Brc1ccc(-c2cccc3c2sc2ccccc23)cc1 ; c1ccc(-c2ccc(N(c3ccc(-c4ccc5c6ccccc6n(-c6ccccc6)c5c4)cc3)c3ccc(-c4cccc5c4sc4ccccc45)cc3)cc2)cc1]

This solid was purified by silica gel column chromatography (developing solvent:hexane:toluene=2:1) to give a solid. The obtained solid was recrystallized with ethyl acetate to give 2.6 g of a white solid with a yield of 94%.

By a train sublimation method, 2.1 g of the obtained solid was purified by sublimation under the conditions where the pressure was 3.1 Pa, the argon flow rate was 15 mL/min, and the temperature was 345° C. After the sublimation purification, 1.8 g of a white solid was obtained at a collection rate of 86%.

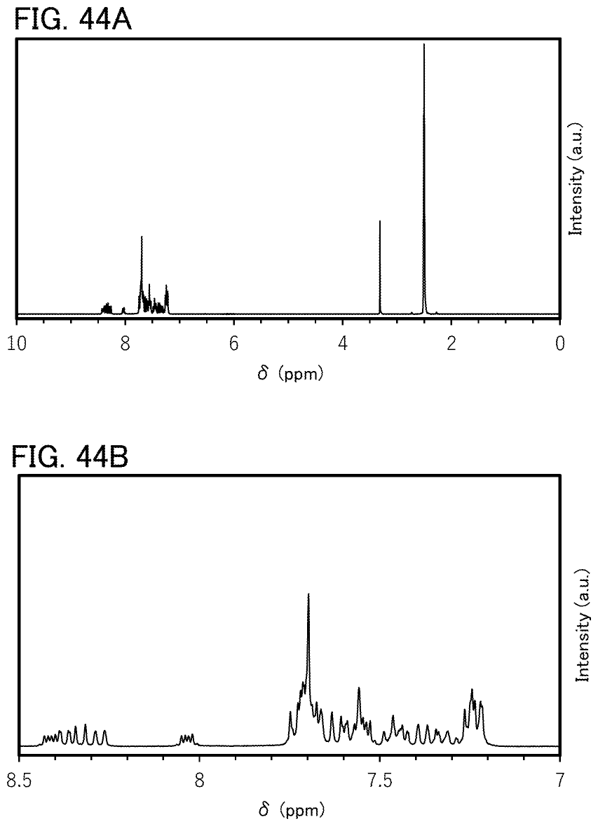

Analysis results by nuclear magnetic resonance spectroscopy (¹H-NMR) of the white solid obtained in the synthesis example are shown in FIG. 44A and FIG. 44B. Note that FIG. 44B is a graph where the range from 7 ppm to 8.5 ppm in FIG. 44A is enlarged. Numerical data is shown below. The results revealed that PCBBiPDBt-02 was synthesized in this synthesis example.

¹H NMR (DMSO-d₆, 300 MHz): δ=7.22-7.39 (m, 9H), 7.42-7.49 (m, 3H), 7.51-7.75 (m, 19H), 8.03 (dd, J1=6.0 Hz, J2=3.3 Hz, 1H), 8.28 (d, J1=7.5 Hz, 1H), 8.33 (d, J1=8.1 Hz, 1H), 8.37 (dd, J1=7.5 Hz, J2=1.2 Hz, 1H), 8.41 (dd, J1=6.0 Hz, J2=3.0 Hz, 1H).

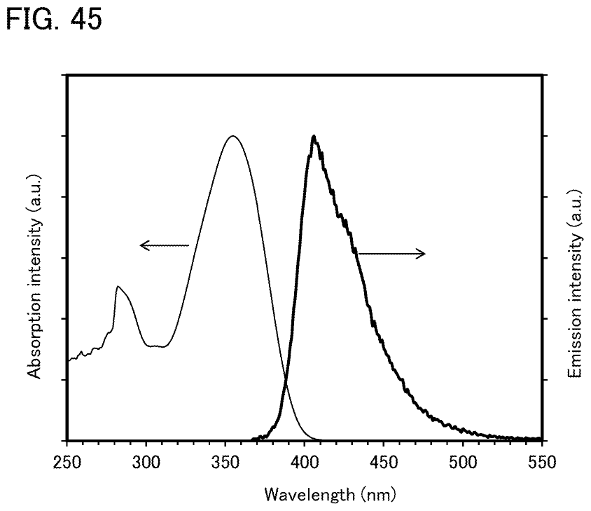

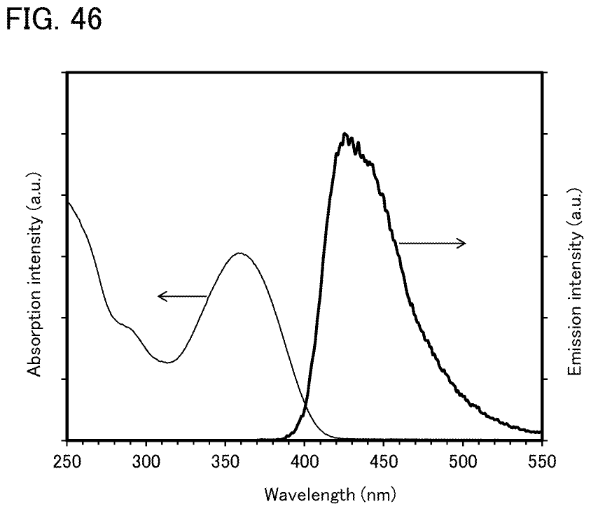

Next, FIG. 45 shows measurement results of an absorption spectrum and an emission spectrum of PCBBiPDBt-02 in a toluene solution, and FIG. 46 shows an absorption spectrum and an emission spectrum of a thin film thereof. In the graphs, the horizontal axis represents wavelength, the vertical axes represent absorbance and emission intensity, the thin solid line represents the absorption spectrum, and the thick solid line represents the emission spectrum. The absorbance shown in FIG. 45 is a result obtained by subtraction of a measured absorption spectrum of only toluene in a quartz cell from a measured absorption spectrum of the toluene solution in a quartz cell.

The solid thin film was formed over a quartz substrate by a vacuum evaporation method. The absorption spectrum of the toluene solution was measured with an ultraviolet-visible light spectrophotometer (V550, manufactured by JASCO Corporation), and the spectrum from which the measured spectrum of toluene alone put in a quartz cell was subtracted was shown. The absorption spectrum of the thin film was measured using a spectrophotometer (U4100 Spectrophotometer, manufactured by Hitachi High-Technologies Corporation). The emission spectrum was measured using a fluorescence spectrophotometer (FS920, manufactured by Hamamatsu Photonics K.K.).

As shown in FIG. 45, PCBBiPDBt-02 in the toluene solution had absorption peaks at 355 nm and 282 nm, and emission wavelength peaks at 406 nm and 426 nm (excitation wavelength: 357 nm). In addition, as shown in FIG. 46, the thin film of PCBBiPDBt-02 had absorption peaks at 359 nm and 290 nm, and an emission wavelength peak at 427 nm (excitation wavelength 360 nm).

Next, PCBBiPDBt-02 obtained in this example was analyzed by liquid chromatography mass spectrometry (abbreviation: LC/MS analysis).

In the LC/MS analysis, LC (liquid chromatography) separation was carried out with Ultimate 3000 produced by Thermo Fisher Scientific K.K., and the MS analysis (mass analysis) was carried out with Q Exactive produced by Thermo Fisher Scientific K.K.

In the LC separation, a given column was used at a column temperature of 40° C., and solution sending was performed in such a manner that an appropriate solvent was selected, the sample was prepared by dissolving PCBBiPDBt-02 in an organic solvent at a given concentration, and the injection amount was 5.0 μL.

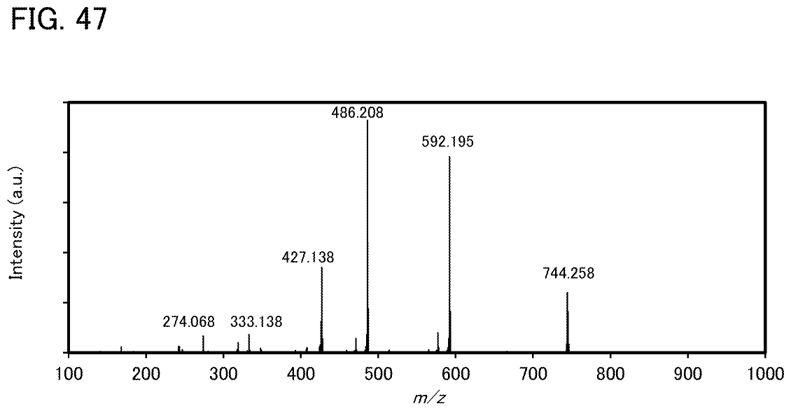

By a Targeted-MS² method, MS² measurement of m/z=744.26, which was an ion derived from PCBBiPDBt-02, was performed. For setting of the Targeted-MS², the mass range of a target ion was set to m/z=744.26±2.0 (isolation window=4) and detection was performed in a positive mode. The measurement was performed with energy NCE (Normalized Collision Energy) for accelerating a target ion in a collision cell set to 50. The obtained MS spectrum is shown in FIG. 47.

It was found from the results in FIG. 47 that, owing to the presence and absence of hydrogen ions and isotopes, precursor ions of PCBBiPDBt-02 were mainly detected at around 744, and product ions of PCBBiPDBt-02 were detected at m/z of around 592, around 486, around 427, around 333, around 274, around 242, and around 168 when the collision energy was 50 eV. The results shown in FIG. 47 can be regarded as important data for identifying PCBBiPDBt-02.

Note that the product ion at around m/z=592 is presumed to be a cation in the state where one biphenyl group was eliminated from PCBBiPDBt-02, which suggests that PCBBiPDBt-02 contains a biphenyl group.

Note that the product ion at around m/z=486 is presumed to be a cation in the state where one 4-(dibenzothiophen-4-yl)phenyl group was eliminated from PCBBiPDBt-02, which suggests that PCBBiPDBt-02 contains a 4-(dibenzothiophen-4-yl)phenyl group.

Note that the product ion at around m/z=427 is presumed to be a cation in the state where one 4-(9-phenyl-9H-carbazol-2-yl)phenyl group was eliminated from PCBBiPDBt-02, which suggests that PCBBiPDBt-02 contains a 4-(9-phenyl-9H-carbazol-2-yl)phenyl group.

### Example 7

### Synthesis example 2

In this example, a synthesis method of 3-(dibenzothiophen-4-yl)-4′-phenyl-4″-(9-phenyl-9H-carbazol-2-yl)triphenylamine (abbreviation: mPCBBiPDBt-02), which is the monoamine compound of one embodiment of the present invention described in Embodiment 1, will be described. The structure of mPCBBiPDBt-02 is shown below.

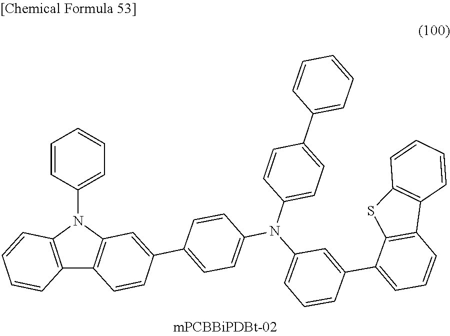 [SMILES: c1ccc(-c2ccc(N(c3ccc(-c4ccc5c6ccccc6n(-c6ccccc6)c5c4)cc3)c3cccc(-c4cccc5c4sc4ccccc45)c3)cc2)cc1]

Into a 200-mL three-neck flask were put 1.8 g (3.7 mmol) of N-(4-biphenyl)-N-[4-(9-phenyl-9H-carbazol-2-yl)phenyl]amine, 1.1 g (3.4 mmol) of 4-(3-bromophenyl)dibenzothiophene, and 0.97 g (10 mmol) of sodium tert-butoxide. To this mixture was added 20 mL of toluene and 0.2 mL of a 10% hexane solution of tri(tert-butyl)phosphine, and this mixture was degassed by being stirred while the pressure was being reduced. To this mixture was added 19 mg (34 μmol) of bis(dibenzylideneacetone)palladium(0), and the mixture was heated and stirred at 110° C. for 8 hours under a nitrogen stream. After the stirring, toluene was added to this mixture, and the resulting mixture was suction-filtered through Florisil, Celite, and alumina to obtain a filtrate. The obtained filtrate was concentrated to give a solid.

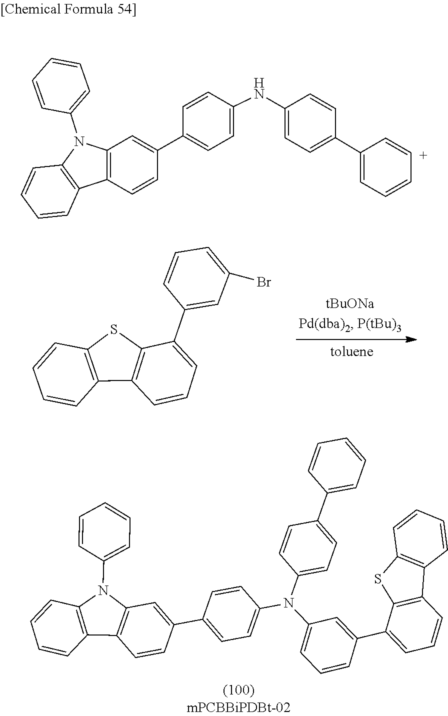 [SMILES: [C+]1=CC(c2ccc(Nc3ccc(-c4ccc5c6ccccc6n(-c6ccccc6)c5c4)cc3)cc2)=CC=C1 ; Brc1cccc(-c2cccc3c2sc2ccccc23)c1 ; c1ccc(-c2ccc(N(c3ccc(-c4ccc5c6ccccc6n(-c6ccccc6)c5c4)cc3)c3cccc(-c4cccc5c4sc4ccccc45)c3)cc2)cc1]

This solid was purified by silica gel column chromatography (developing solvent:hexane:toluene=2:1) to give a solid.

The obtained solid was recrystallized with toluene/ethyl acetate to give 2.3 g of a white solid with a yield of 91%.

By a train sublimation method, 2.3 g of the obtained solid was purified by sublimation. The sublimation purification was performed by heating at 335° C. under conditions where the pressure was 3.0 Pa and the argon flow rate was 15 mL/min.

After the sublimation purification, 1.6 g of a white solid was obtained at a collection rate of 69%.

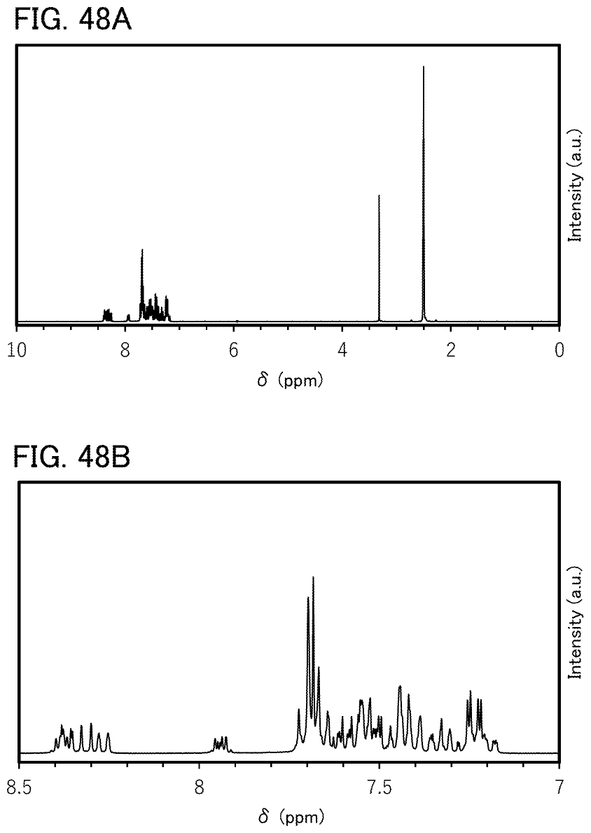

Analysis results by nuclear magnetic resonance spectroscopy (¹H-NMR) of the white solid obtained in the synthesis example are shown in FIG. 48A and FIG. 48B. Note that FIG. 48B is a graph where the range from 7 ppm to 8.5 ppm in FIG. 48A is enlarged. Numerical data is shown below. The results revealed that mPCBBiPDBt-02 was synthesized in this synthesis example.

¹H NMR (DMSO-d₆, 300 MHz): δ=7.17-7.72 (m, 31H), 7.91-7.97 (m, 1H), 8.27 (d, J1=7.5 Hz, 1H), 8.31 (d, J1=8.1 Hz, 1H), 8.35-8.41 (m, 2H).

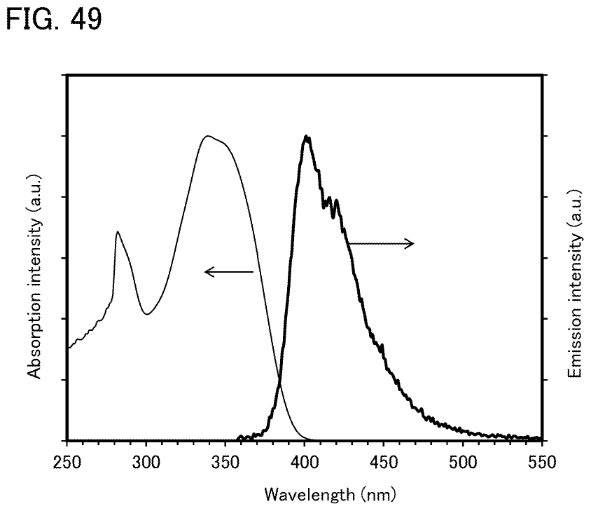

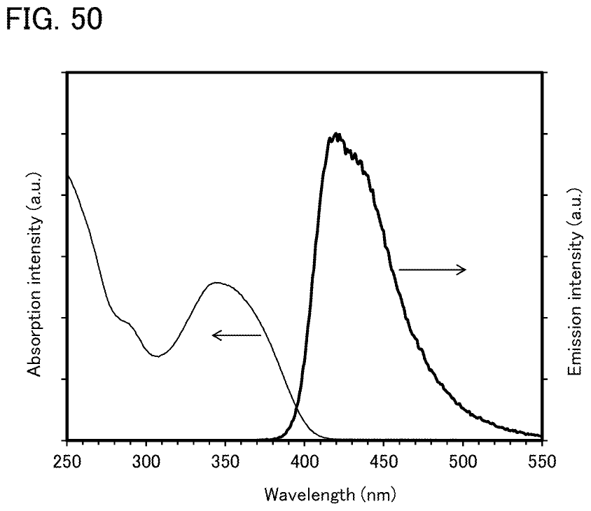

Next, FIG. 49 shows measurement results of an absorption spectrum and an emission spectrum of mPCBBiPDBt-02 in a toluene solution. FIG. 50 shows an absorption spectrum and an emission spectrum of a thin film thereof. In the graphs, the horizontal axis represents wavelength, the vertical axes represent absorbance and emission intensity, the thin solid line represents the absorption spectrum, and the thick solid line represents the emission spectrum. The absorbance shown in FIG. 49 is a result obtained by subtraction of a measured absorption spectrum of only toluene in a quartz cell from a measured absorption spectrum of the toluene solution in a quartz cell.

The solid thin film was formed over a quartz substrate by a vacuum evaporation method. The absorption spectrum of the toluene solution was measured with an ultraviolet-visible light spectrophotometer (V550, manufactured by JASCO Corporation), and the spectrum from which the measured spectrum of toluene alone put in a quartz cell was subtracted was shown. The absorption spectrum of the thin film was measured using a spectrophotometer (U4100 Spectrophotometer, manufactured by Hitachi High-Technologies Corporation). The emission spectrum was measured using a fluorescence spectrophotometer (FS920, manufactured by Hamamatsu Photonics K.K.).

As shown in FIG. 49, mPCBBiPDBt-02 in the toluene solution had absorption peaks at 339 nm and 282 nm, and emission wavelength peaks at 401 nm and 420 nm (excitation wavelength: 348 nm). In addition, as shown in FIG. 50, the thin film of mPCBBiPDBt-02 had absorption peaks at 344 nm and 288 nm, and an emission wavelength peak at 420 nm (excitation wavelength 360 nm).

Next, mPCBBiPDBt-02 obtained in this example was analyzed by liquid chromatography mass spectrometry (abbreviation: LC/MS analysis).

In the LC/MS analysis, LC (liquid chromatography) separation was carried out with Ultimate 3000 produced by Thermo Fisher Scientific K.K., and the MS analysis (mass analysis) was carried out with Q Exactive produced by Thermo Fisher Scientific K.K.

In the LC separation, a given column was used at a column temperature of 40° C., and solution sending was performed in such a manner that an appropriate solvent was selected, the sample was prepared by dissolving mPCBBiPDBt-02 in an organic solvent at a given concentration, and the injection amount was 5.0 μL.

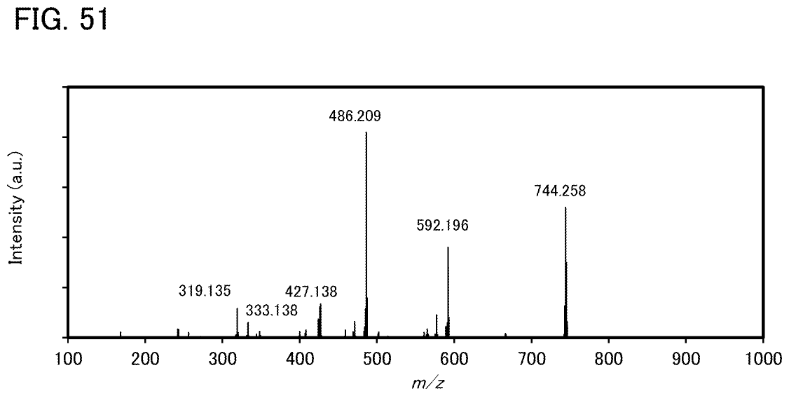

By a Targeted-MS² method, MS² measurement of m/z=744.26, which was an ion derived from mPCBBiPDBt-02, was performed. For setting of the Targeted-MS², the mass range of a target ion was set to m/z=744.26±2.0 (isolation window=4) and detection was performed in a positive mode. The measurement was performed with energy NCE (Normalized Collision Energy) for accelerating a target ion in a collision cell set to 50. The obtained MS spectrum is shown in FIG. 51.

It was found from the results in FIG. 51 that, owing to the presence and absence of hydrogen ions and isotopes, precursor ions of mPCBBiPDBt-02 were mainly detected at around 744, and product ions of mPCBBiPDBt-02 were detected at m/z of around 592, around 486, around 427, around 333, around 319, around 242, and around 168 when the collision energy was 50 eV. The results shown in FIG. 51 can be regarded as important data for identifying mPCBBiPDBt-02.

Note that the product ion at around m/z=592 is presumed to be a cation in the state where one biphenyl group was eliminated from mPCBBiPDBt-02, which suggests that mPCBBiPDBt-02 contains a biphenyl group.

Note that the product ion at around m/z=486 is presumed to be a cation in the state where one 3-(dibenzothiophen-4-yl)phenyl group was eliminated from mPCBBiPDBt-02, which suggests that mPCBBiPDBt-02 contains a 3-(dibenzothiophen-4-yl)phenyl group.

Note that the product ion at around m/z=427 is presumed to be a cation in the state where one 4-(9-phenyl-9H-carbazol-2-yl)phenyl group was eliminated from mPCBBiPDBt-02, which suggests that mPCBBiPDBt-02 contains a 4-(9-phenyl-9H-carbazol-2-yl)phenyl group.

### Example 8

### Synthesis example 3

In this example, a synthesis method of 4-[3-(dibenzothiophen-4-yl)phenyl]-4′-phenyl-4″-(9-phenyl-9H-carbazol-2-yl)triphenylamine (abbreviation: pmPCBBiBPDBt-02), which is the monoamine compound of one embodiment of the present invention described in Embodiment 1, will be described. The structure of pmPCBBiBPDBt-02 is shown below.

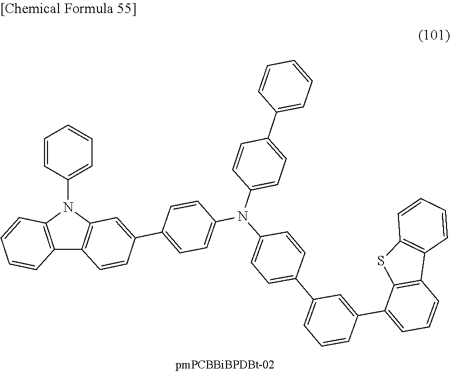 [SMILES: c1ccc(-c2ccc(N(c3ccc(-c4cccc(-c5cccc6c5sc5ccccc56)c4)cc3)c3ccc(-c4ccc5c6ccccc6n(-c6ccccc6)c5c4)cc3)cc2)cc1]

Into a 200-mL three-neck flask were put 1.6 g (3.4 mmol) of N-(4-biphenyl)-N-[4-(9-phenyl-9H-carbazol-2-yl)phenyl]amine, 1.3 g (3.0 mmol) of 4-(4′-bromo[1,1′-biphenyl]-3-yl)dibenzothiophene, and 0.88 g (9.1 mmol) of sodium tert-butoxide. To this mixture was added 20 mL of toluene and 0.2 mL of a 10% hexane solution of tri(tert-butyl)phosphine, and this mixture was degassed by being stirred while the pressure was being reduced. To this mixture was added 17 mg (30 μmol) of bis(dibenzylideneacetone)palladium(0), and the mixture was heated and stirred at 110° C. for 12 hours under a nitrogen stream.

After the stirring, toluene was added to this mixture, and the resulting mixture was suction-filtered through Florisil, Celite, and alumina to obtain a filtrate. The obtained filtrate was concentrated to give a solid. The synthesis scheme of this synthesis example is shown below.

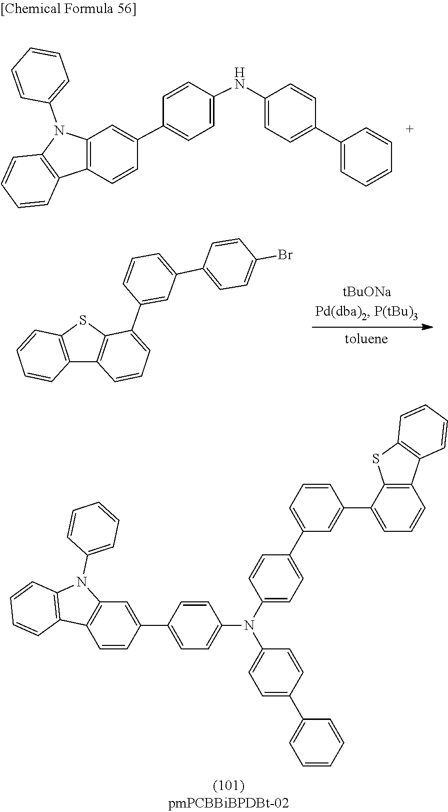 [SMILES: c1ccc(-c2ccc(Nc3ccc(-c4ccc5c6ccccc6n(-c6ccccc6)c5c4)cc3)cc2)cc1 ; Brc1ccc(-c2cccc(-c3cccc4c3sc3ccccc34)c2)cc1 ; c1ccc(-c2ccc(N(c3ccc(-c4cccc(-c5cccc6c5sc5ccccc56)c4)cc3)c3ccc(-c4ccc5c6ccccc6n(-c6ccccc6)c5c4)cc3)cc2)cc1]

This solid was purified by silica gel column chromatography (developing solvent: hexane:toluene=2:1) to give a solid. The obtained solid was reprecipitated with toluene/ethanol to give 2.3 g of a solid with a yield of 91%.

By a train sublimation method, 2.3 g of the obtained solid was purified by sublimation. The sublimation purification was performed by heating at 385° C. under conditions where the pressure was 3.0 Pa and the argon flow rate was 15 mL/min. After the sublimation purification, 2.0 g of a white solid was obtained at a collection rate of 85%.

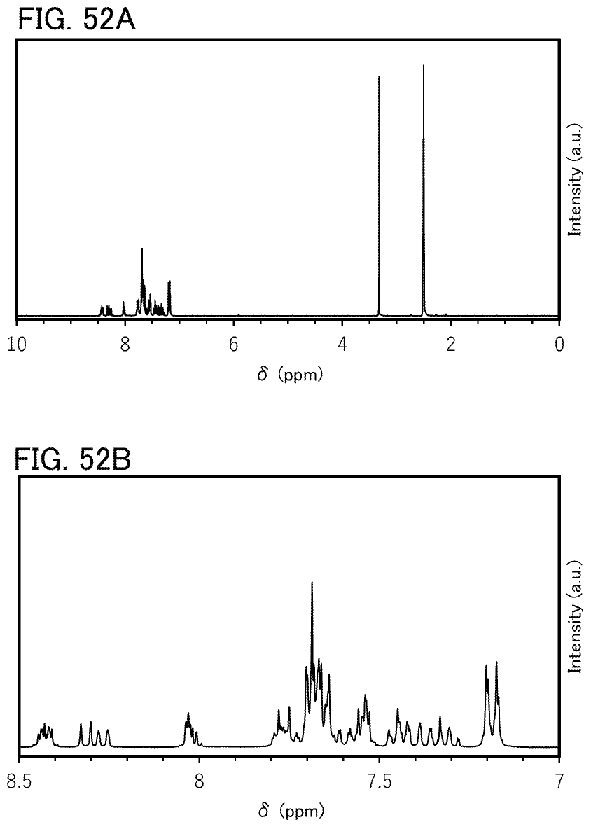

Analysis results by nuclear magnetic resonance spectroscopy (¹H-NMR) of the white solid obtained in this synthesis example are shown in FIG. 52A and FIG. 52B. Note that FIG. 52B is a graph where the range from 7 ppm to 8.5 ppm in FIG. 52A is enlarged. Numerical data is shown below. The results revealed that pmPCBBiBPDBt-02 was synthesized in this synthesis example.

¹H NMR (DMSO-d₆, 300 MHz): δ=7.17-7.20 (m, 6H), 7.28-7.47 (m, 6H), 7.51-7.80 (m, 22H), 7.99-8.05 (m, 2H), 8.27 (d, J1=7.5 Hz, 1H), 8.32 (d, J1=8.4 Hz, 1H), 8.39-8.46 (m, 2H).

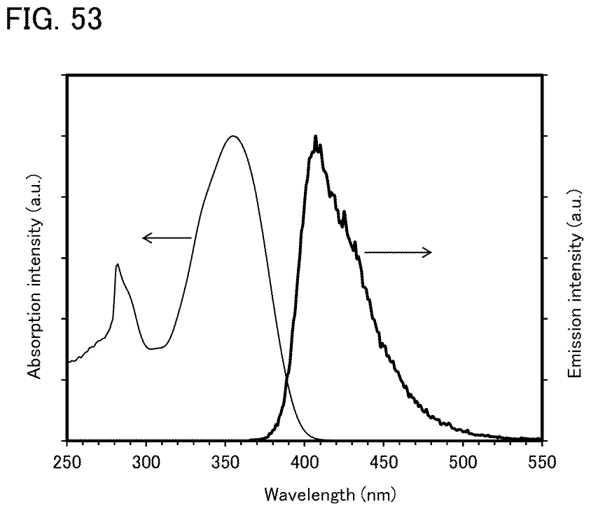

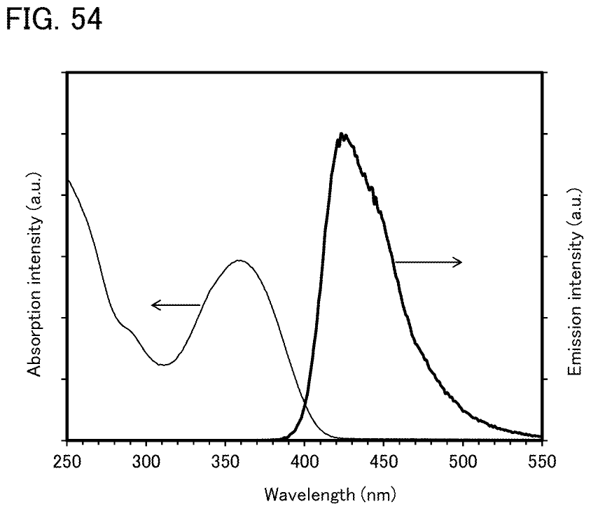

Next, FIG. 53 shows measurement results of an absorption spectrum and an emission spectrum of pmPCBBiBPDBt-02 in a toluene solution, and FIG. 54 shows an absorption spectrum and an emission spectrum of a thin film thereof. In the graphs, the horizontal axis represents wavelength, the vertical axes represent absorbance and emission intensity, the thin solid line represents the absorption spectrum, and the thick solid line represents the emission spectrum. The absorbance shown in FIG. 53 is a result obtained by subtraction of a measured absorption spectrum of only toluene in a quartz cell from a measured absorption spectrum of the toluene solution in a quartz cell.

The solid thin film was formed over a quartz substrate by a vacuum evaporation method. The absorption spectrum of the toluene solution was measured with an ultraviolet-visible light spectrophotometer (V550, manufactured by JASCO Corporation), and the spectrum from which the measured spectrum of toluene alone put in a quartz cell was subtracted was shown. The absorption spectrum of the thin film was measured using a spectrophotometer (U4100 Spectrophotometer, manufactured by Hitachi High-Technologies Corporation). The emission spectrum was measured using a fluorescence spectrophotometer (FS920, manufactured by Hamamatsu Photonics K.K.).

As shown in FIG. 53, pmPCBBiBPDBt-02 in the toluene solution had absorption peaks at 355 nm, 298 nm, and 282 nm, and an emission wavelength peak at 407 nm (excitation wavelength: 356 nm). In addition, as shown in FIG. 54, the thin film of pmPCBBiBPDBt-02 had absorption peaks at 358 nm and 289 nm, and an emission wavelength peak at 425 nm (excitation wavelength 360 nm).

Next, pmPCBBiBPDBt-02 obtained in this example was analyzed by liquid chromatography mass spectrometry (abbreviation: LC/MS analysis).

In the LC/MS analysis, LC (liquid chromatography) separation was carried out with Ultimate 3000 produced by Thermo Fisher Scientific K.K., and the MS analysis (mass analysis) was carried out with Q Exactive produced by Thermo Fisher Scientific K.K.

In the LC separation, a given column was used at a column temperature of 40° C., and solution sending was performed in such a manner that an appropriate solvent was selected, the sample was prepared by dissolving pmPCBBiBPDBt-02 in an organic solvent at a given concentration, and the injection amount was 5.0 μL.

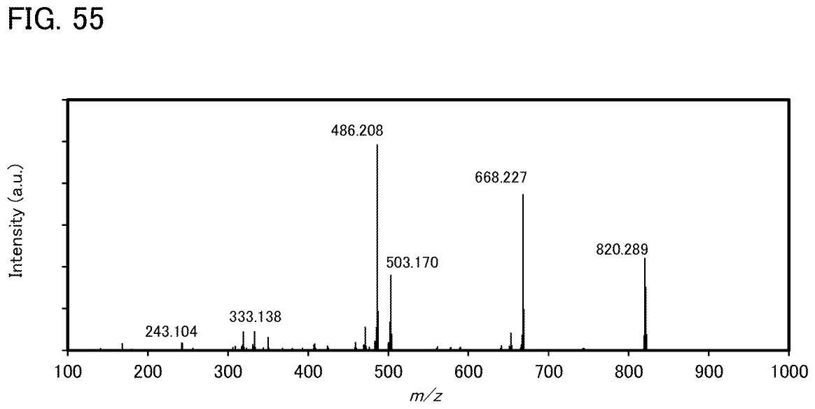

By a Targeted-MS² method, MS² measurement of m/z=820.29, which was an ion derived from pmPCBBiBPDBt-02, was performed. For setting of the Targeted-MS², the mass range of a target ion was set to m/z=820.29±2.0 (isolation window=4) and detection was performed in a positive mode. The measurement was performed with energy NCE (Normalized Collision Energy) for accelerating a target ion in a collision cell set to 50. The obtained MS spectrum is shown in FIG. 55.

It was found from the results in FIG. 55 that, owing to the presence and absence of hydrogen ions and isotopes, precursor ions of pmPCBBiBPDBt-02 were mainly detected at around 820, and product ions of pmPCBBiBPDBt-02 were detected at m/z of around 668, around 503, around 486, around 408, around 333, around 243, and around 168 when the collision energy was 50 eV. The results shown in FIG. 55 can be regarded as important data for identifying pmPCBBiBPDBt-02.

Note that the product ion at around m/z=668 is presumed to be a cation in the state where one biphenyl group was eliminated from pmPCBBiBPDBt-02, which suggests that pmPCBBiBPDBt-02 contains a biphenyl group.

Note that the product ion at around m/z=503 is presumed to be a cation in the state where one 4-(9-phenyl-9H-carbazol-2-yl)phenyl group was eliminated from pmPCBBiBPDBt-02, which suggests that pmPCBBiBPDBt-02 contains a 4-(9-phenyl-9H-carbazol-2-yl)phenyl group.

Note that the product ion at around m/z=486 is presumed to be a cation in the state where one 4-[3-(dibenzothiophen-4-yl)phenyl]phenyl group was eliminated from pmPCBBiBPDBt-02, which suggests that pmPCBBiBPDBt-02 contains a 4-[3-(dibenzothiophen-4-yl)phenyl]phenyl group.

### Example 9

### Synthesis example 4

In this example, a synthesis method of 4-[3-(dibenzothiophen-4-yl)phenyl]-4′-phenyl-4″-(9-phenyl-9H-carbazol-3-yl)triphenylamine (abbreviation: pmPCBBiBPDBt), which is the monoamine compound of one embodiment of the present invention described in Embodiment 1, will be described. The structure of pmPCBBiBPDBt is shown below.

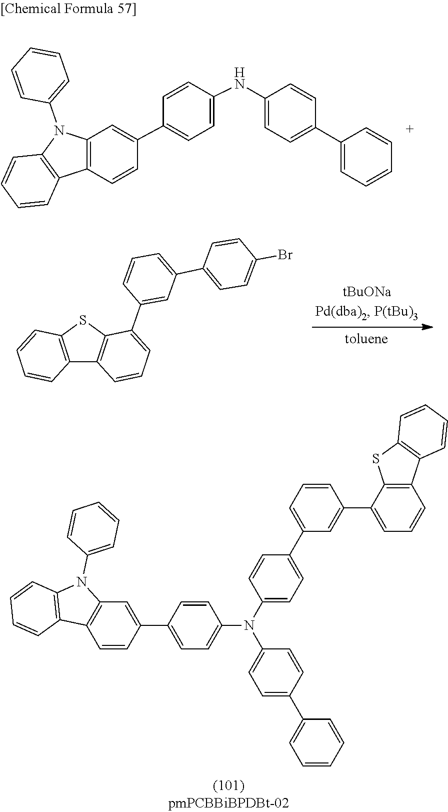 [SMILES: c1ccc(-c2ccc(Nc3ccc(-c4ccc5c6ccccc6n(-c6ccccc6)c5c4)cc3)cc2)cc1 ; Brc1ccc(-c2cccc(-c3cccc4c3sc3ccccc34)c2)cc1 ; c1ccc(-c2ccc(N(c3ccc(-c4cccc(-c5cccc6c5sc5ccccc56)c4)cc3)c3ccc(-c4ccc5c6ccccc6n(-c6ccccc6)c5c4)cc3)cc2)cc1]

Into a 200-mL three-neck flask were put 1.6 g (3.4 mmol) of N-biphenyl-[4-(9-phenyl-9H-carbazol-3-yl)phenyl]amine, 1.3 g (3.0 mmol) of 4-[3-(4-bromophenyl)phenyl]dibenzothiophene, and 0.88 g (9.1 mmol) of sodium tert-butoxide. To this mixture was added 20 mL of toluene and 0.2 mL of a 10% hexane solution of tri(tert-butyl)phosphine, and this mixture was degassed by being stirred while the pressure was being reduced. To this mixture was added 17 mg (30 μmol) of bis(dibenzylideneacetone)palladium(0), and the mixture was heated and stirred at 120° C. for 7.5 hours under a nitrogen stream.

After the stirring, toluene was added to this mixture, and the resulting mixture was suction-filtered through Florisil, Celite, and alumina to obtain a filtrate. The obtained filtrate was concentrated to give a solid. The synthesis scheme of this synthesis example is shown below.

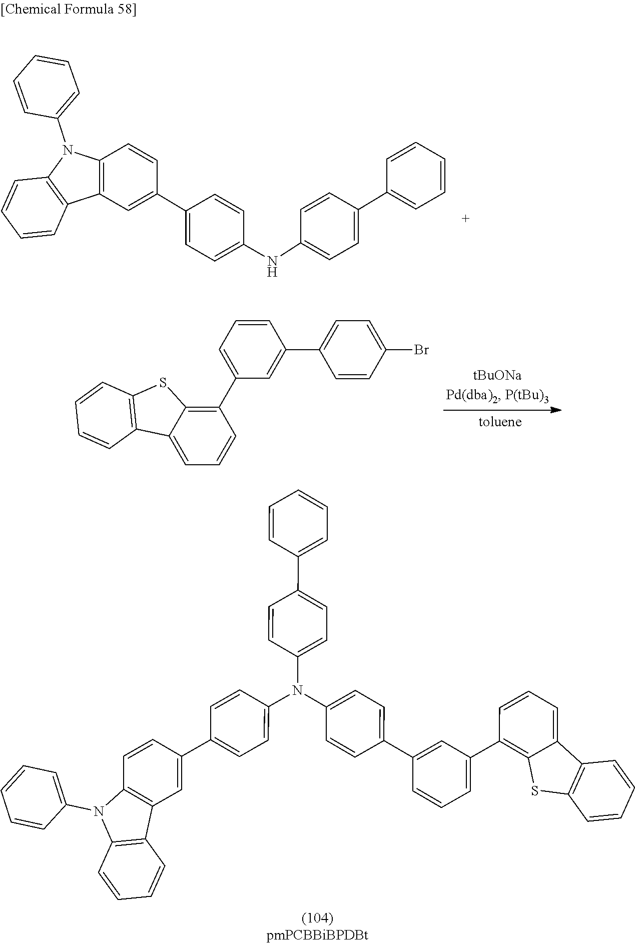 [SMILES: c1ccc(-c2ccc(Nc3ccc(-c4ccc5c(c4)c4ccccc4n5-c4ccccc4)cc3)cc2)cc1 ; Brc1ccc(-c2cccc(-c3cccc4c3sc3ccccc34)c2)cc1 ; c1ccc(-c2ccc(N(c3ccc(-c4cccc(-c5cccc6c5sc5ccccc56)c4)cc3)c3ccc(-c4ccc5c(c4)c4ccccc4n5-c4ccccc4)cc3)cc2)cc1]

This solid was purified by silica gel column chromatography (developing solvent: hexane:toluene=2:1 and then hexane:toluene=3:2) to give a solid. The obtained solid was recrystallized with toluene/ethyl acetate to give 2.2 g of a solid with a yield of 86%.

By a train sublimation method, 2.0 g of the obtained solid was purified by sublimation. The sublimation purification was performed by heating at 380° C. under conditions where the pressure was 3.2 Pa and the argon flow rate was 15 mL/min. After the sublimation purification, 1.7 g of a pale yellow solid was obtained at a collection rate of 85%.

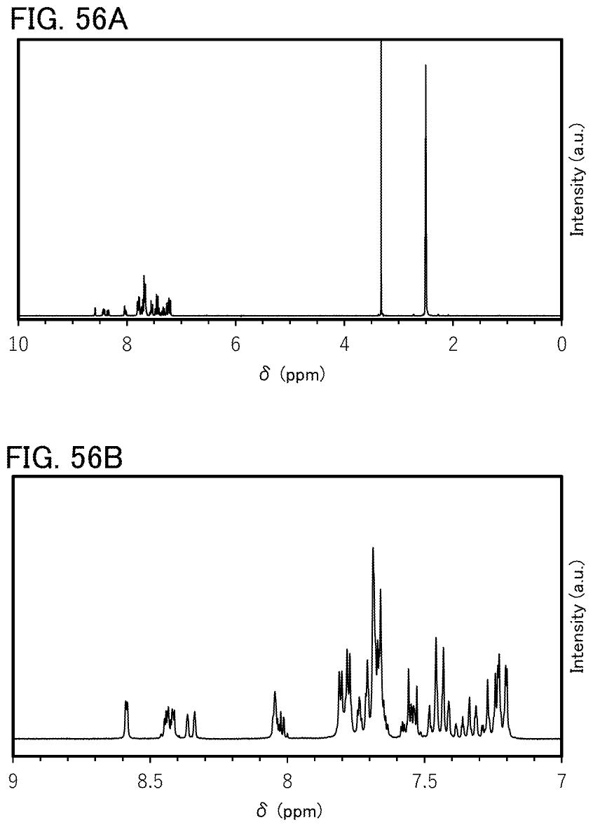

Analysis results by nuclear magnetic resonance spectroscopy (¹H-NMR) of the white solid obtained in this synthesis example are shown in FIG. 56A and FIG. 56B. Note that FIG. 56B is a graph where the range from 7 ppm to 9 ppm in FIG. 56A is enlarged. Numerical data is shown below. The results revealed that pmPCBBiBPDBt was synthesized in this synthesis example.

¹H NMR (DMSO-d₆, 300 MHz): δ=7.20-7.48 (m, 13H), 7.51-7.59 (m, 3H), 7.63-7.81 (m, 18H), 8.00-8.05 (m, 2H), 8.35 (d, J1=7.5 Hz, 1H), 8.40-8.46 (m, 2H), 8.59 (d, J1=1.5 Hz, 1H).

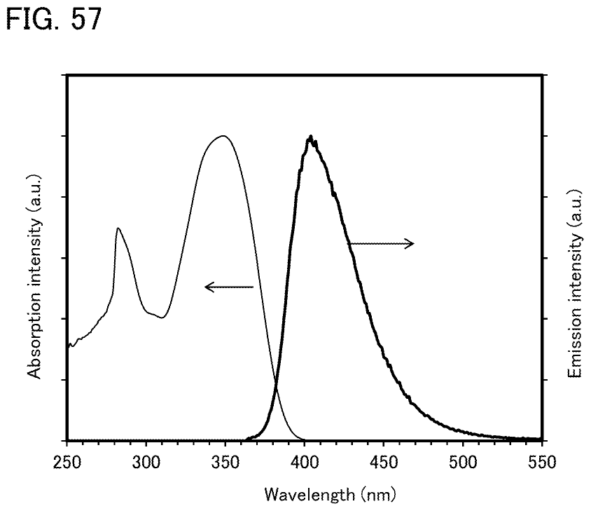

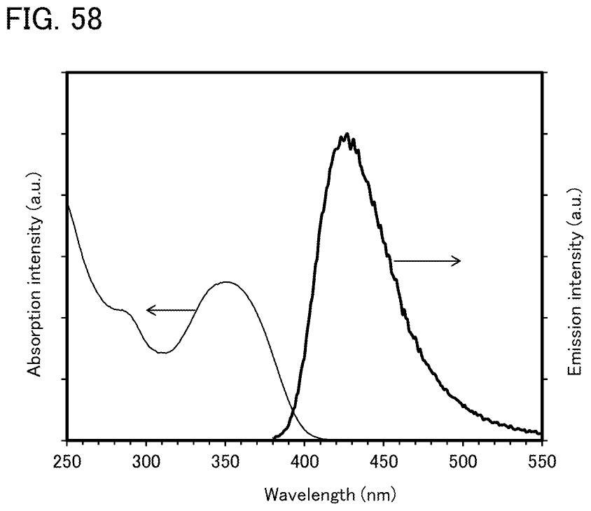

Next, FIG. 57 shows measurement results of an absorption spectrum and an emission spectrum of pmPCBBiBPDBt in a toluene solution. FIG. 58 shows an absorption spectrum and an emission spectrum of a thin film thereof. In the graphs, the horizontal axis represents wavelength, the vertical axes represent absorbance and emission intensity, the thin solid line represents the absorption spectrum, and the thick solid line represents the emission spectrum. The absorbance shown in FIG. 57 is a result obtained by subtraction of a measured absorption spectrum of only toluene in a quartz cell from a measured absorption spectrum of the toluene solution in a quartz cell.

The solid thin film was formed over a quartz substrate by a vacuum evaporation method. The absorption spectrum of the toluene solution was measured with an ultraviolet-visible light spectrophotometer (V550, manufactured by JASCO Corporation), and the spectrum from which the measured spectrum of toluene alone put in a quartz cell was subtracted was shown. The absorption spectrum of the thin film was measured using a spectrophotometer (U4100 Spectrophotometer, manufactured by Hitachi High-Technologies Corporation). The emission spectrum was measured using a fluorescence spectrophotometer (FS920, manufactured by Hamamatsu Photonics K.K.).

As shown in FIG. 57, pmPCBBiBPDBt in the toluene solution had absorption peaks at 349 nm and 282 nm, and an emission wavelength peak at 404 nm (excitation wavelength: 354 nm). In addition, as shown in FIG. 58, the thin film of pmPCBBiBPDBt had absorption peaks at 351 nm and 285 nm, and an emission wavelength peak at 425 nm (excitation wavelength 370 nm).

### Example 10

### Synthesis Example 5

In this example, a synthesis method of 4-[3-(dibenzofuran-4-yl)phenyl]-4′-phenyl-4″-(9-phenyl-9H-carbazol-2-yl)triphenylamine (abbreviation: pmPCBBiBPDBf-02), which is the monoamine compound of one embodiment of the present invention described in Embodiment 1, will be described. The structure of pmPCBBiBPDBf-02 is shown below.

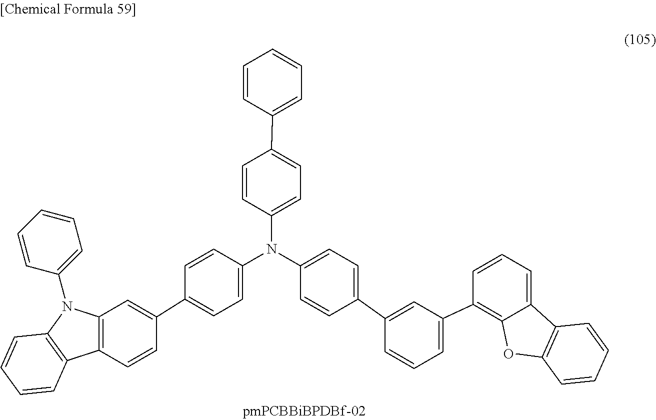 [SMILES: c1ccc(-c2ccc(N(c3ccc(-c4cccc(-c5cccc6c5oc5ccccc56)c4)cc3)c3ccc(-c4ccc5c6ccccc6n(-c6ccccc6)c5c4)cc3)cc2)cc1]

Into a 200-mL three-neck flask were put 1.7 g (3.4 mmol) of N-biphenyl-[4-(9-phenyl-9H-carbazol-2-yl)phenyl]amine, 1.2 g (3.1 mmol) of 4-[3-(4-bromophenyl)phenyl]dibenzofuran, and 0.90 g (9.3 mmol) of sodium tert-butoxide. To this mixture was added 20 mL of toluene and 0.2 mL of a 10% hexane solution of tri(tert-butyl)phosphine, and this mixture was degassed by being stirred while the pressure was being reduced. To this mixture was added 18 mg (31 μmol) of bis(dibenzylideneacetone)palladium(0), and the mixture was heated and stirred at 120° C. for 7 hours under a nitrogen stream.

After the stirring, toluene was added to this mixture, and the resulting mixture was suction-filtered through Florisil, Celite, and alumina to obtain a filtrate. The obtained filtrate was concentrated to give a solid. The synthesis scheme of this synthesis example is shown below.

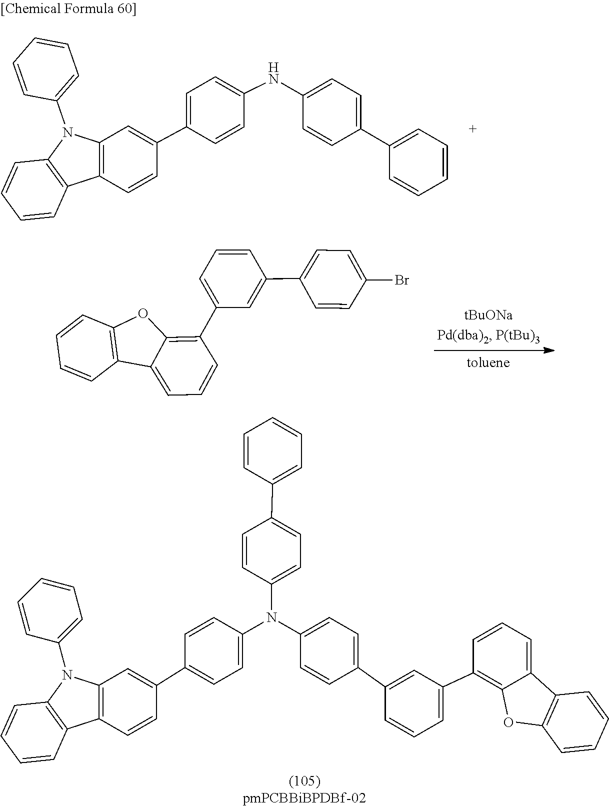 [SMILES: c1ccc(-c2ccc(Nc3ccc(-c4ccc5c6ccccc6n(-c6ccccc6)c5c4)cc3)cc2)cc1 ; Brc1ccc(-c2cccc(-c3cccc4c3oc3ccccc34)c2)cc1 ; c1ccc(-c2ccc(N(c3ccc(-c4cccc(-c5cccc6c5oc5ccccc56)c4)cc3)c3ccc(-c4ccc5c6ccccc6n(-c6ccccc6)c5c4)cc3)cc2)cc1]

This solid was purified by silica gel column chromatography (developing solvent: hexane:toluene=2:1) to give a solid. The obtained solid was reprecipitated with ethyl acetate/ethanol to give 2.2 g of a solid with a yield of 87%.

By a train sublimation method, 2.1 g of the obtained solid was purified by sublimation. The sublimation purification was performed by heating at 370° C. under conditions where the pressure was 3.3 Pa and the argon flow rate was 15 mL/min. After the sublimation purification, 1.9 g of a white solid was obtained at a collection rate of 87%.

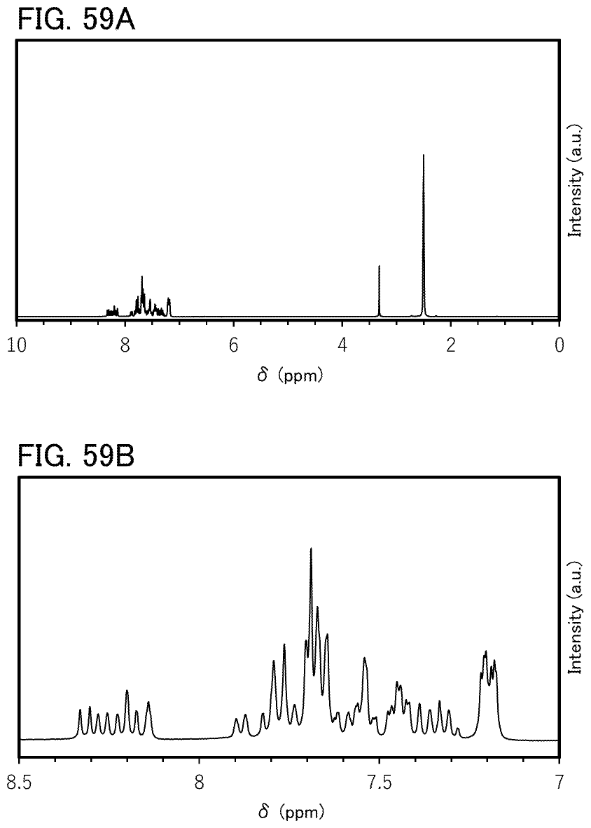

Analysis results by nuclear magnetic resonance spectroscopy (¹H-NMR) of the white solid obtained in this synthesis example are shown in FIG. 59A and FIG. 59B. Note that FIG. 59B is a graph where the range from 7 ppm to 8.5 ppm in FIG. 59A is enlarged. Numerical data is shown below. The results revealed that pmPCBBiBPDBf-02 was synthesized in this synthesis example.

¹H NMR (DMSO-d₆, 300 MHz): δ=7.18-7.22 (m, 6H), 7.28-7.82 (m, 28H), 7.88 (d, J1=7.5 Hz, 1H), 8.14-8.28 (m, 4H), 8.32 (d, J1=8.4 Hz, 1H).

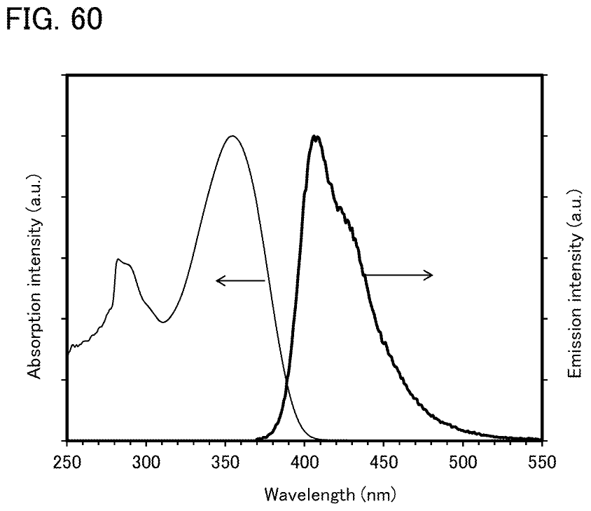

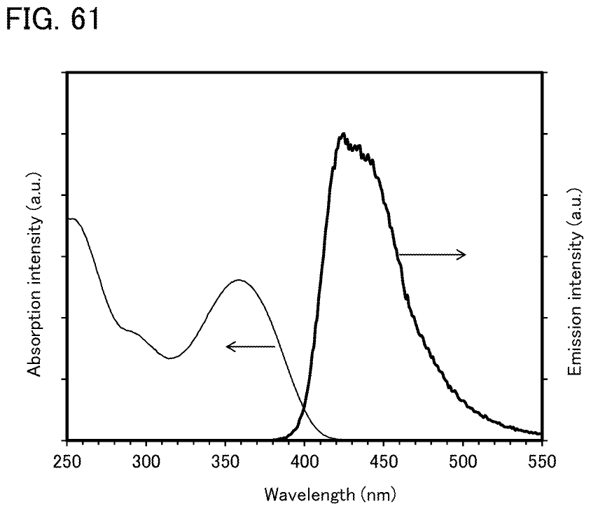

Next, FIG. 60 shows measurement results of an absorption spectrum and an emission spectrum of pmPCBBiBPDBf-02 in a toluene solution. FIG. 61 shows an absorption spectrum and an emission spectrum of a thin film thereof. In the graphs, the horizontal axis represents wavelength, the vertical axes represent absorbance and emission intensity, the thin solid line represents the absorption spectrum, and the thick solid line represents the emission spectrum. The absorbance shown in FIG. 60 is a result obtained by subtraction of a measured absorption spectrum of only toluene in a quartz cell from a measured absorption spectrum of the toluene solution in a quartz cell.

The solid thin film was formed over a quartz substrate by a vacuum evaporation method. The absorption spectrum of the toluene solution was measured with an ultraviolet-visible light spectrophotometer (V550, manufactured by JASCO Corporation), and the spectrum from which the measured spectrum of toluene alone put in a quartz cell was subtracted was shown. The absorption spectrum of the thin film was measured using a spectrophotometer (U4100 Spectrophotometer, manufactured by Hitachi High-Technologies Corporation). The emission spectrum was measured using a fluorescence spectrophotometer (FS920, manufactured by Hamamatsu Photonics K.K.).

As shown in FIG. 60, pmPCBBiBPDBf-02 in the toluene solution had absorption peaks at 354 nm and 282 nm, and emission wavelength peaks at 407 nm and 423 nm (excitation wavelength: 360 nm). In addition, as shown in FIG. 61, the thin film of pmPCBBiBPDBf-02 had absorption peaks at 358 nm, 293 nm, and 255 nm, and emission wavelength peaks at 424 nm and 440 nm (excitation wavelength 370 nm).

### Example 11

### Synthesis Example 6

In this example, a synthesis method of N-biphenyl-4-yl-N-[3-(dibenzothiophen-4-yl)biphenyl-4-yl]-9-phenyl-9H-carbazol-2-amine (abbreviation: pmPCBiBPDBt-02), which is the monoamine compound of one embodiment of the present invention described in Embodiment 1, will be described. The structure of pmPCBiBPDBt-02 is shown below.

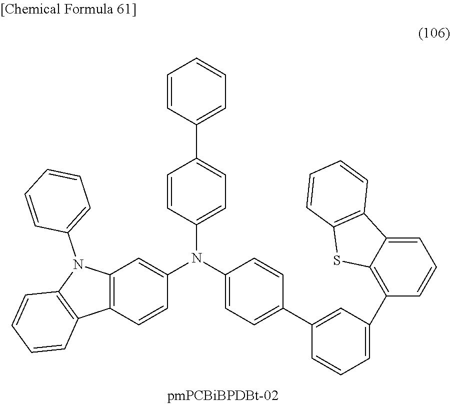 [SMILES: c1ccc(-c2ccc(N(c3ccc(-c4cccc(-c5cccc6c5sc5ccccc56)c4)cc3)c3ccc4c5ccccc5n(-c5ccccc5)c4c3)cc2)cc1]

Into a 200-mL three-neck flask were put 1.9 g (3.7 mmol) of 4-phenyl-4′-[3-(dibenzothiophen-2-yl)phenyl]diphenylamine, 1.1 g (3.4 mmol) of 2-bromo-9-phenyl-9H-carbazole, and 1.1 g (11 mmol) of sodium tert-butoxide. To this mixture was added 20 mL of toluene and 0.2 mL of a 10% hexane solution of tri(tert-butyl)phosphine, and this mixture was degassed by being stirred while the pressure was being reduced. To this mixture was added 19 mg (34 μmol) of bis(dibenzylideneacetone)palladium(0), and the mixture was heated and stirred at 120° C. for 7 hours under a nitrogen stream. After the stirring, toluene was added to this mixture, and the resulting mixture was suction-filtered through Florisil, Celite, and alumina to obtain a filtrate. The filtrate was concentrated to give a solid. The synthesis scheme of this synthesis example is shown below.

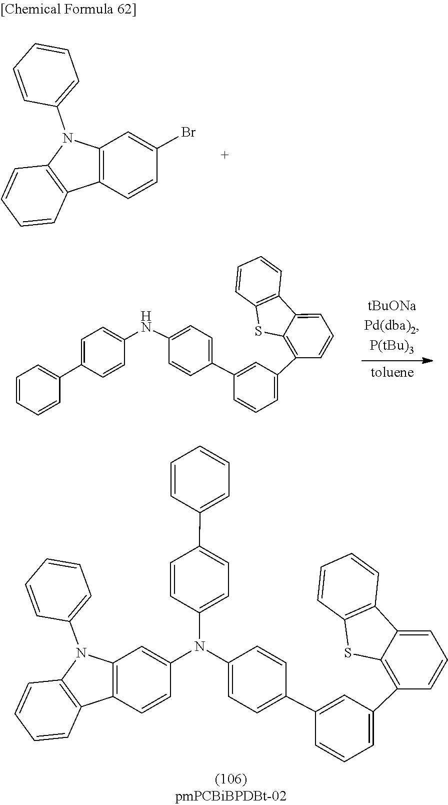 [SMILES: Brc1ccc2c3ccccc3n(-c3ccccc3)c2c1 ; c1ccc(-c2ccc(Nc3ccc(-c4cccc(-c5cccc6c5sc5ccccc56)c4)cc3)cc2)cc1 ; c1ccc(-c2ccc(N(c3ccc(-c4cccc(-c5cccc6c5sc5ccccc56)c4)cc3)c3ccc4c5ccccc5n(-c5ccccc5)c4c3)cc2)cc1]

This solid was purified by silica gel column chromatography (developing solvent: hexane:toluene=2:1) to give a solid. The obtained solid was reprecipitated with ethyl acetate/ethanol to give 2.1 g of a solid with a yield of 85%.

By a train sublimation method, 2.1 g of the obtained solid was purified by sublimation. The sublimation purification was performed by heating at 345° C. under conditions where the pressure was 3.4 Pa and the argon flow rate was 15 mL/min. After the sublimation purification, 1.9 g of a pale yellow solid was obtained at a collection rate of 88%.

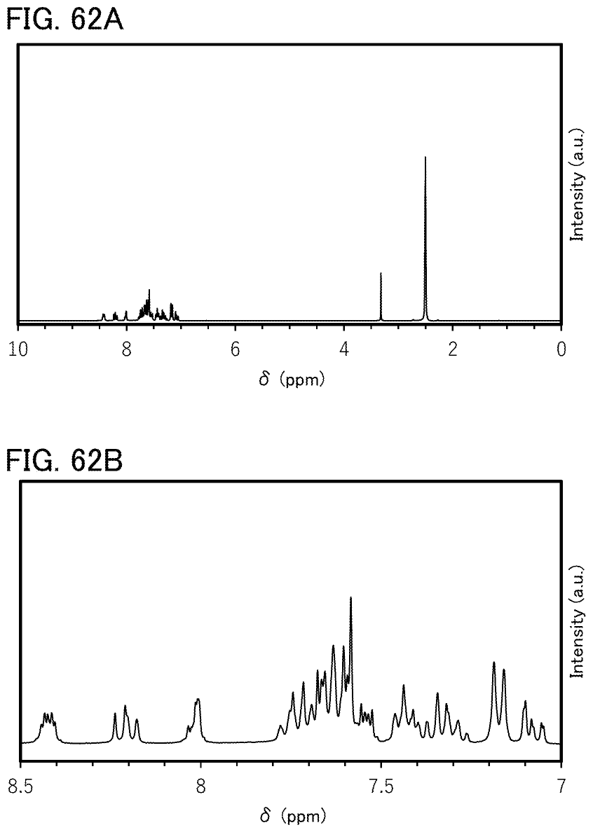

Analysis results by nuclear magnetic resonance spectroscopy (¹H-NMR) of the white solid obtained in this synthesis example are shown in FIG. 62A and FIG. 62B. Note that FIG. 62B is a graph where the range from 7 ppm to 8.5 ppm in FIG. 62A is enlarged. Numerical data is shown below. The results revealed that pmPCBiBPDBt-02 was synthesized in this synthesis example.

¹H NMR (DMSO-d₆, 300 MHz): δ=7.05-7.10 (m, 2H), 7.17 (d, J1=8.1 Hz, 4H), 7.26-7.46 (m, 7H), 7.51-7.78 (m, 17H), 7.99-8.04 (m, 2H), 8.18-8.24 (m, 2H), 8.39-8.45 (m, 2H).

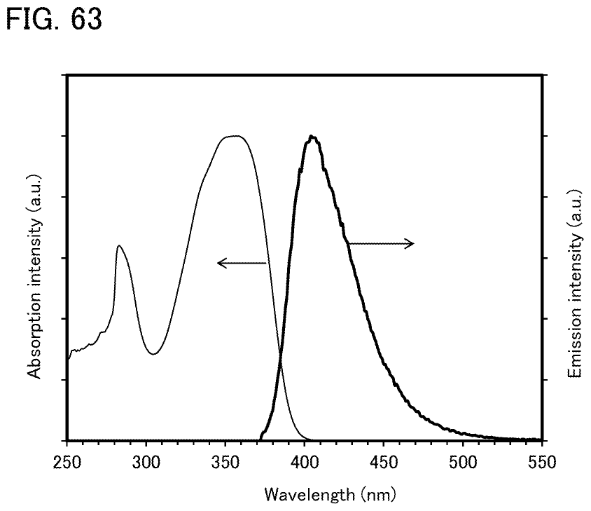

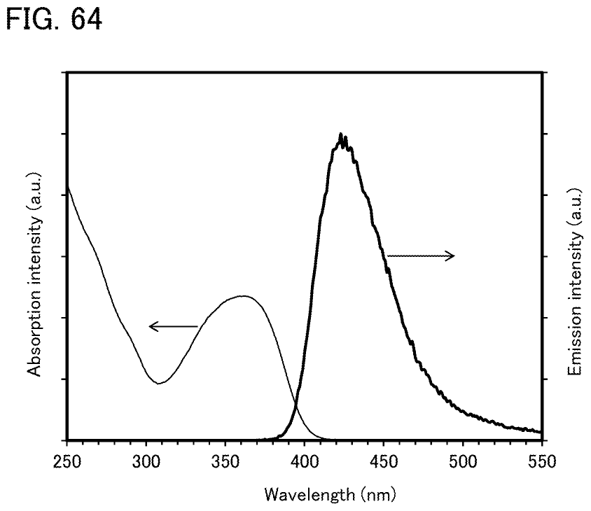

Next, FIG. 63 shows measurement results of an absorption spectrum and an emission spectrum of pmPCBiBPDBt-02 in a toluene solution. FIG. 64 shows an absorption spectrum and an emission spectrum of a thin film thereof. In the graphs, the horizontal axis represents wavelength, the vertical axes represent absorbance and emission intensity, the thin solid line represents the absorption spectrum, and the thick solid line represents the emission spectrum. The absorbance shown in FIG. 63 is a result obtained by subtraction of a measured absorption spectrum of only toluene in a quartz cell from a measured absorption spectrum of the toluene solution in a quartz cell.

The solid thin film was formed over a quartz substrate by a vacuum evaporation method. The absorption spectrum of the toluene solution was measured with an ultraviolet-visible light spectrophotometer (V550, manufactured by JASCO Corporation), and the spectrum from which the measured spectrum of toluene alone put in a quartz cell was subtracted was shown. The absorption spectrum of the thin film was measured using a spectrophotometer (U4100 Spectrophotometer, manufactured by Hitachi High-Technologies Corporation). The emission spectrum was measured using a fluorescence spectrophotometer (FS920, manufactured by Hamamatsu Photonics K.K.).

As shown in FIG. 63, pmPCBiBPDBt-02 in the toluene solution had absorption peaks at 357 nm and 283 nm, and an emission wavelength peak at 405 nm (excitation wavelength: 363 nm). In addition, as shown in FIG. 64, the thin film of pmPCBiBPDBt-02 had absorption peaks at 362 nm, 292 nm, and 270 nm, and an emission wavelength peak at 423 nm (excitation wavelength 363 nm).

### Example 12

### Synthesis Example 7

In this example, a synthesis method of N-(3′,5′-ditertiarybutyl-1,1′-biphenyl-4-yl)-N-(1,1′-biphenyl-2-yl)-9,9-dimethyl-9H-fluoren-2-amine (abbreviation: mmtBuBioFBi), which is the organic compound of one embodiment of the present invention described in Embodiment 1, will be described. The structure of mmtBuBioFBi is shown below.

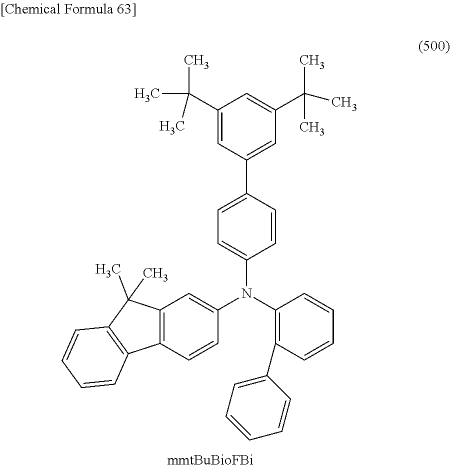 [SMILES: CC(C)(C)c1cc(-c2ccc(N(c3ccc4c(c3)C(C)(C)c3ccccc3-4)c3ccccc3-c3ccccc3)cc2)cc(C(C)(C)C)c1]

In a three-neck flask were put 2.22 g (7.4 mmol) of 4-chloro-3′,5′-di-tert-butyl-1,1′-biphenyl, 2.94 g (8.1 mmol) of 2-(2-biphenylyl)amino-9,9-dimethylfluorene, 2.34 g (24.4 mmol) of sodium-tert-butoxide, and 37 mL of xylene, the mixture was degassed under reduced pressure, and then the air in the flask was replaced with nitrogen. To this mixture, 107.6 mg (0.31 mmol) of di-t-butyl(1-methyl-2,2-diphenylcyclopropyl)phosphine (abbreviation: cBRIDP (registered trademark)) and 28.1 mg (0.077 mmol) of allylpalladium chloride dimer were added. This mixture was heated at 100° C. for approximately 4 hours. After that, the temperature of the flask was lowered to approximately 70° C., and approximately 4 mL of water was added, so that a solid was precipitated. The precipitated solid was separated by filtration. The filtrate was concentrated, and the obtained solution was purified by silica gel column chromatography. The obtained solution was concentrated, ethanol was added thereto, and the obtained solution was concentrated again; this process was performed three times to obtain an ethanol suspension, and recrystallization was performed. The precipitate was cooled to approximately −10° C. and then filtrated, and the obtained solid was dried at approximately 130° C. under reduced pressure, whereby 2.07 g of a target white solid was obtained in a yield of 45%. The synthesis scheme of this synthesis example is shown below.

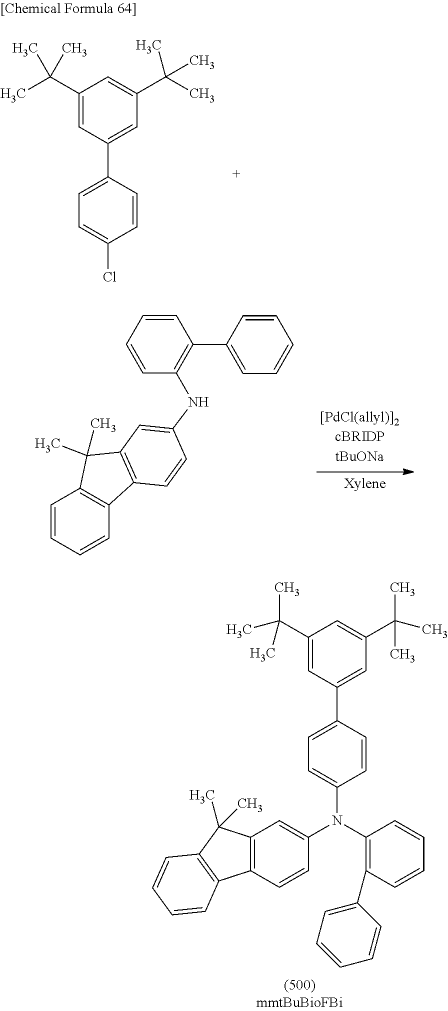 [SMILES: CC(C)(C)c1cc(-c2ccc(Cl)cc2)cc(C(C)(C)C)c1 ; CC1(C)c2ccccc2-c2ccc(Nc3ccccc3-c3ccccc3)cc21 ; CC(C)(C)c1cc(-c2ccc(N(c3ccc4c(c3)C(C)(C)c3ccccc3-4)c3ccccc3-c3ccccc3)cc2)cc(C(C)(C)C)c1]

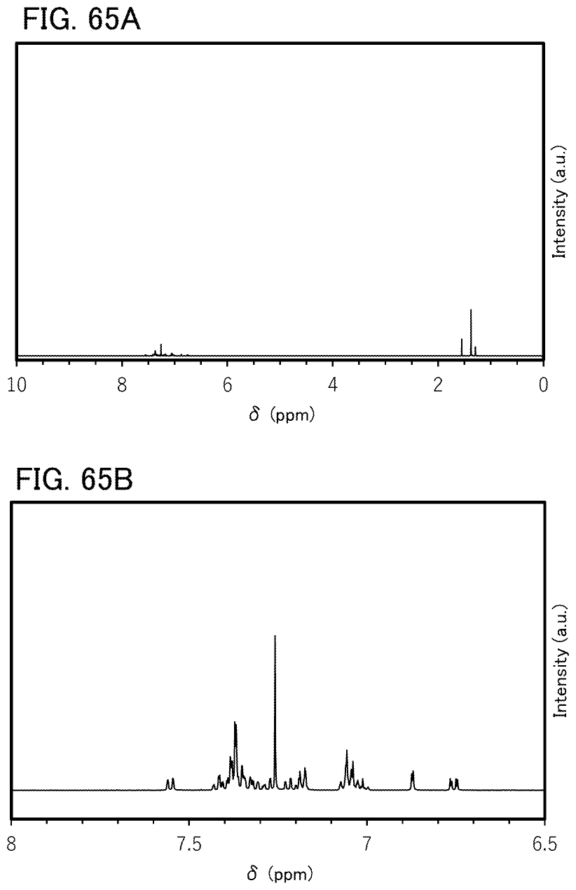

FIG. 65A and FIG. 65B show analysis results by nuclear magnetic resonance spectroscopy (¹H-NMR) of the white solid obtained in this synthesis example. Note that FIG. 65B is a graph where the range from 6.5 ppm to 8 ppm in FIG. 65A is enlarged. In addition, numerical data is shown below. The results showed that mmtBuBioFBi was synthesized in this synthesis example.

¹H NMR (CDCl₃, 500 MHz): δ=1.29 (s, 6H), 1.38 (s, 18H), 6.76 (dd, J₁=8.0 Hz, J₂=2.0 Hz, 1H), 6.87 (d, J=2.5 Hz, 1H), 7.00-7.08 (m, 5H), 7.18-7.23 (m, 3H), 7.27-7.43 (m, 12H), 7.55 (d, J=7.5 Hz, 1H).

Then, 2.0 g of the obtained solid was purified by a train sublimation method. The purification by sublimation was conducted by heating at 225° C. under the conditions where the pressure was 3.77 Pa and the argon flow rate was 15.0 mL/min. After the purification by sublimation, 1.9 g of a white solid was obtained at a collection rate of 95%.

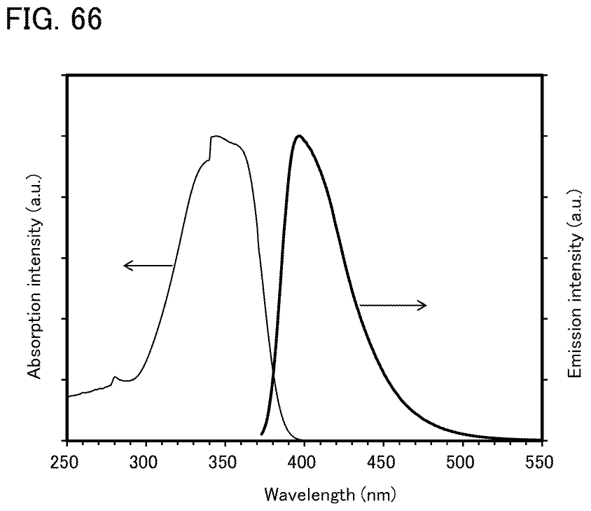

Next, FIG. 66 shows measurement results of an absorption spectrum and an emission spectrum of mmtBuBioFBi in a toluene solution. In the graph, the horizontal axis represents wavelength, the vertical axes represent absorbance and emission intensity, the thin solid line represents the absorption spectrum, and the thick solid line represents the emission spectrum. The absorbance shown in FIG. 66 is a result obtained by subtraction of a measured absorption spectrum of only toluene in a quartz cell from a measured absorption spectrum of the toluene solution in a quartz cell.

The absorption spectrum of the toluene solution was measured with an ultraviolet-visible light spectrophotometer (V550, manufactured by JASCO Corporation), and the spectrum from which the measured spectrum of toluene alone put in a quartz cell was subtracted was shown. The emission spectrum was measured using a fluorescence spectrophotometer (FP-8600, manufactured by JASCO Corporation).

As shown in FIG. 66, mmtBuBioFBi in the toluene solution had an absorption peak at 344 nm, and an emission wavelength peak at 397 nm (excitation wavelength: 344 nm).

The refractive index of mmtBuBioFBi was measured with a spectroscopic ellipsometer (M-2000U manufactured by J.A. Woollam Japan Corp.). For the measurement, films obtained by depositing the materials of the respective layers to approximately 50 nm over a quartz substrate by a vacuum evaporation method were used.

The results showed that mmtBuBioFBi was a material with a low refractive index: the ordinary refractive index was within the range higher than or equal to 1.50 and lower than or equal to 1.75 in the entire blue light emission region (greater than or equal to 455 nm and less than or equal to 465 nm), and the ordinary refractive index at 633 nm was within the range higher than or equal to 1.45 and lower than or equal to 1.70.

Next, Tg of mmtBuBioFBi was measured. The Tg was measured with a differential scanning calorimeter (PYRIS 1 DSC manufactured by PerkinElmer Japan Co., Ltd.) while a powder was put on an aluminum cell. As a result, the Tg of mmtBuBioFBi was 100° C.

### REFERENCE NUMERALS

101: anode, 102: cathode, 103: EL layer, 111: hole-injection layer, 112: hole-transport layer, 113: light-emitting layer, 114: electron-transport layer, 115: electron-injection layer, 116: charge generation layer, 117: p-type layer, 118: electron-relay layer, 119: electron-injection buffer layer, 120: hole-transport region, 400: substrate, 401: anode, 403: EL layer, 404: cathode, 405: sealant, 406: sealant, 407: sealing substrate, 412: pad, 420: IC chip, 601: driver circuit portion (source line driver circuit), 602: pixel portion, 603: driver circuit portion (gate line driver circuit), 604: sealing substrate, 605: sealant, 607: space, 608: wiring, 609: FPC (flexible print circuit), 610: element substrate, 611: switching FET, 612: current controlling FET, 613: anode, 614: insulator, 616: EL layer, 617: cathode, 618: light-emitting device, 951: substrate, 952: electrode, 953: insulating layer, 954: partition layer, 955: EL layer, 956: electrode, 1001: substrate, 1002: base insulating film, 1003: gate insulating film, 1006: gate electrode, 1007: gate electrode, 1008: gate electrode, 1020: first interlayer insulating film, 1021: second interlayer insulating film, 1022: electrode, 1024W: anode, 1024R: anode, 1024G: anode, 1024B: anode, 1025: partition, 1028: EL layer, 1029: cathode, 1031: sealing substrate, 1032: sealant, 1033: transparent base material, 1034R: red coloring layer, 1034G: green coloring layer, 1034B: blue coloring layer, 1035: black matrix, 1036: overcoat layer, 1037: third interlayer insulating film, 1040: pixel portion, 1041: driver circuit portion, 1042: peripheral portion, 2001: housing, 2002: light source, 2100: robot, 2110: arithmetic unit, 2101: illuminance sensor, 2102: microphone, 2103: upper camera, 2104: speaker, 2105: display, 2106: lower camera, 2107: obstacle sensor, 2108: moving mechanism, 3001: lighting device, 5000: housing, 5001: display portion, 5002: display portion, 5003: speaker, 5004: LED lamp, 5006: connection terminal, 5007: sensor, 5008: microphone, 5012: support portion, 5013: earphone, 5100: cleaning robot, 5101: display, 5102: camera, 5103: brush, 5104: operation button, 5150: portable information terminal, 5151: housing, 5152: display region, 5153: bend portion, 5120: dust, 5200: display region, 5201: display region, 5202: display region, 5203: display region, 7101: housing, 7103: display portion, 7105: stand, 7107: display portion, 7109: operation key, 7110: remote controller, 7201: main body, 7202: housing, 7203: display portion, 7204: keyboard, 7205: external connection port, 7206: pointing device, 7210: display portion, 7401: housing, 7402: display portion, 7403: operation button, 7404: external connection port, 7405: speaker, 7406: microphone, 9310: portable information terminal, 9311: display panel, 9313: hinge, 9315: housing

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. A light-emitting device comprising:
an anode;
a cathode; and
an EL layer positioned between the anode and the cathode,
wherein the EL layer comprises a light-emitting layer, a first layer, and a second layer,
wherein the first layer is positioned between the anode and the light-emitting layer,
wherein the first layer is in contact with the second layer,
wherein the second layer comprises a first organic compound having an arylamine structure,
wherein a first group, a second group, and a third group are bonded to a nitrogen atom comprised in the amine in the first organic compound,
wherein the first group is a group comprising a carbazole structure,
wherein the second group is a group comprising a dibenzofuran structure or a dibenzothiophene structure,
wherein the third group comprises an aromatic hydrocarbon structure having 6 to 18 carbon atoms or a heteroaromatic hydrocarbon structure having 4 to 26 carbon atoms, and
wherein a refractive index of the first layer is lower than a refractive index of the light-emitting layer.

43. The light-emitting device according to claim 42, wherein an ordinary refractive index of the first layer with respect to light with a wavelength greater than or equal to 455 nm and less than or equal to 465 nm is higher than or equal to 1.5 and lower than or equal to 1.75.

44. The light-emitting device according to claim 42, wherein an ordinary refractive index of the first layer with respect to light with a wavelength of 633 nm is higher than or equal to 1.45 and lower than or equal to 1.70.

45. The light-emitting device according to claim 42,
wherein the first layer comprises an organic compound having a hole-transport property, and
wherein an ordinary refractive index of the organic compound having a hole-transport property with respect to light with a wavelength greater than or equal to 455 nm and less than or equal to 465 nm is higher than or equal to 1.5 and lower than or equal to 1.75.

46. The light-emitting device according to claim 42,
wherein the first layer comprises an organic compound having a hole-transport property, and
wherein an ordinary refractive index of the organic compound having a hole-transport property with respect to light with a wavelength of 633 nm is higher than or equal to 1.45 and lower than or equal to 1.70.

47. The light-emitting device according to claim 45, wherein the organic compound having a hole-transport property comprises a plurality of alkyl groups.

48. The light-emitting device according to claim 46, wherein the organic compound having a hole-transport property comprises a plurality of alkyl groups.

49. The light-emitting device according to claim 42,
wherein the carbazole structure of the first group comprises a bonding site at any of a 2-position, a 3-position, and a 9-position, and
wherein the carbazole structure is bonded to the nitrogen atom through the bonding site or the bonding site and a divalent aromatic hydrocarbon group.

50. The light-emitting device according to claim 42,
wherein the carbazole structure of the first group comprises a bonding site at a 2-position or a 3-position, and
wherein the carbazole structure is bonded to the nitrogen atom through the bonding site or the bonding site and a divalent aromatic hydrocarbon group.

51. The light-emitting device according to claim 42,
wherein the carbazole structure of the first group comprises a bonding site at a 2-position, and
wherein the carbazole structure is bonded to the nitrogen atom through the bonding site or the bonding site and a divalent aromatic hydrocarbon group.

52. The light-emitting device according to claim 49,
wherein the divalent aromatic hydrocarbon group is a phenylene group.

53. The light-emitting device according to claim 50,
wherein the divalent aromatic hydrocarbon group is a phenylene group.

54. The light-emitting device according to claim 51,
wherein the divalent aromatic hydrocarbon group is a phenylene group.

55. The light-emitting device according to claim 42,
wherein the dibenzofuran structure or the dibenzothiophene structure of the second group is bonded to the nitrogen atom through a divalent aromatic hydrocarbon group.

56. The light-emitting device according to claim 55,
wherein the divalent aromatic hydrocarbon group comprised in the second group is a phenylene group or a biphenyldiyl group.

57. The light-emitting device according to claim 56,
wherein a positional relationship between bonding sites of the phenylene group or bonding sites of at least one benzene structure of the biphenyldiyl group is a meta-position.

58. The light-emitting device according to claim 42,
wherein the third group is a biphenyl group or a terphenyl group.

59. The light-emitting device according to claim 42,
wherein the third group is a group comprising a dibenzofuran structure or a dibenzothiophene structure.

60. The light-emitting device according to claim 42,
wherein the second layer is positioned between the first layer and the light-emitting layer.

61. A light-emitting device comprising:
an anode;
a cathode; and
an EL layer positioned between the anode and the cathode,
wherein the EL layer comprises a light-emitting layer, a first layer, and a second layer,
wherein the first layer is positioned between the anode and the light-emitting layer,
wherein the first layer is in contact with the second layer,
wherein a refractive index of the first layer is lower than a refractive index of the light-emitting layer, and
wherein the second layer comprises an organic compound represented by General Formula (G1) below: 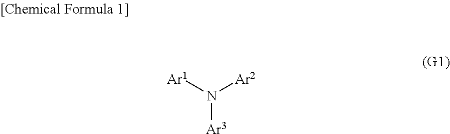
wherein Ar¹ is a group represented by General Formula (g1) below; Ar² is a group represented by General Formula (g2) or (g3) below; and Ar³ is any of the group represented by General Formula (g1) below and an aromatic hydrocarbon group having 6 to 18 carbon atoms, 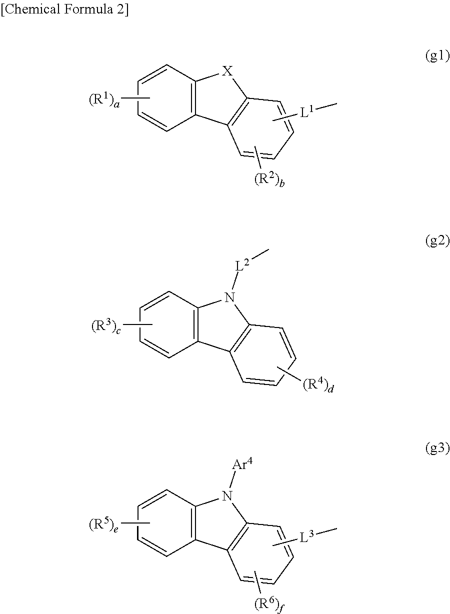
wherein each of R¹ to R⁶ is independently any of a hydrocarbon group having 1 to 6 carbon atoms and an aromatic hydrocarbon group having 6 to 13 carbon atoms; Ar⁴ is a substituted or unsubstituted phenyl group; each of a, c, d, and e independently represents an integer of 0 to 4; each of b and f independently represents an integer of 0 to 3; each of L¹ to L³ independently represents a divalent aromatic hydrocarbon group having 6 to 12 carbon atoms; and X is an oxygen atom or a sulfur atom.

62. The light-emitting device according to claim 61, wherein an ordinary refractive index of the first layer with respect to light with a wavelength greater than or equal to 455 nm and less than or equal to 465 nm is higher than or equal to 1.5 and lower than or equal to 1.75.

63. The light-emitting device according to claim 61, wherein an ordinary refractive index of the first layer with respect to light with a wavelength of 633 nm is higher than or equal to 1.45 and lower than or equal to 1.70.

64. The light-emitting device according to claim 61,
wherein the first layer comprises a second organic compound having a hole-transport property, and
wherein an ordinary refractive index of the organic compound having a hole-transport property with respect to light with a wavelength greater than or equal to 455 nm and less than or equal to 465 nm is higher than or equal to 1.5 and lower than or equal to 1.75, and

65. The light-emitting device according to claim 61,
wherein the first layer comprises a second organic compound having a hole-transport property,
wherein a refractive index of the second organic compound having a hole-transport property with respect to light with a wavelength of 633 nm is higher than or equal to 1.45 and lower than or equal to 1.70.

66. The light-emitting device according to claim 64, wherein the second organic compound having a hole-transport property comprises a plurality of alkyl groups.

67. The light-emitting device according to claim 65, wherein the second organic compound having a hole-transport property comprises a plurality of alkyl groups.

68. The light-emitting device according to claim 61, wherein the X is a sulfur atom.

69. The light-emitting device according to claim 61,
wherein the L1 is any of groups represented by Structural Formulae (L-1) to (L-7) below.

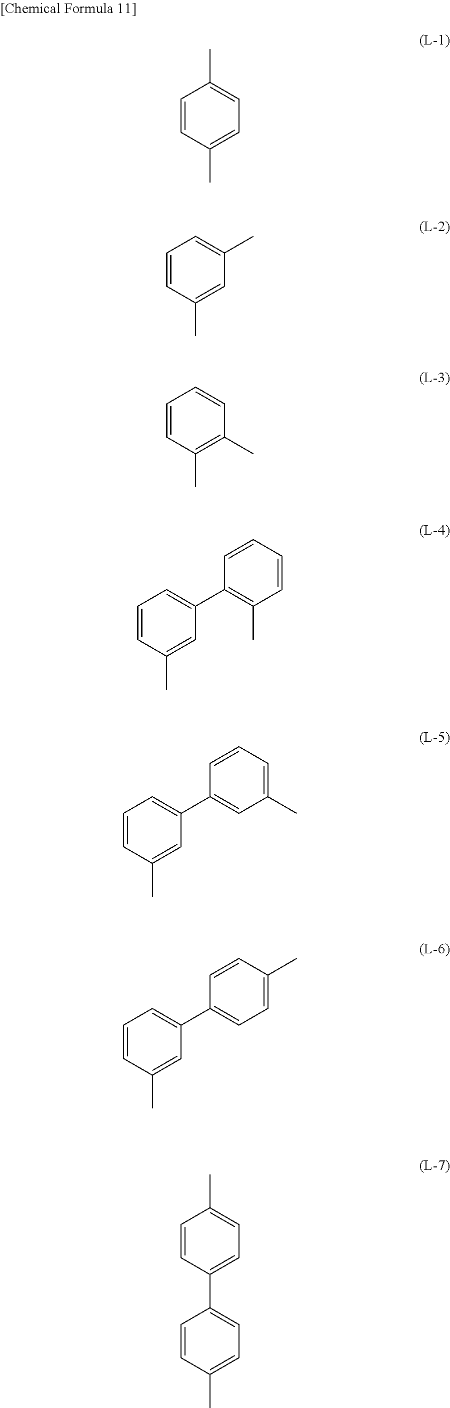

70. The light-emitting device according to claim 61,
wherein the L1 is a group represented by Structural Formula (L-2) or (L-6) below: 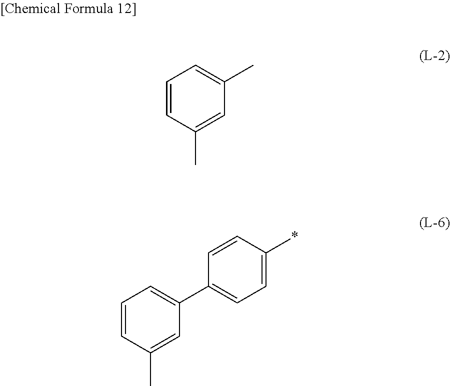
wherein (L-6) is bonded to a nitrogen atom at a position indicated by an asterisk.

71. The light-emitting device according to claim 61,
wherein the Ar2 is a group represented by General Formula (g3-1) or (g3-2) below: 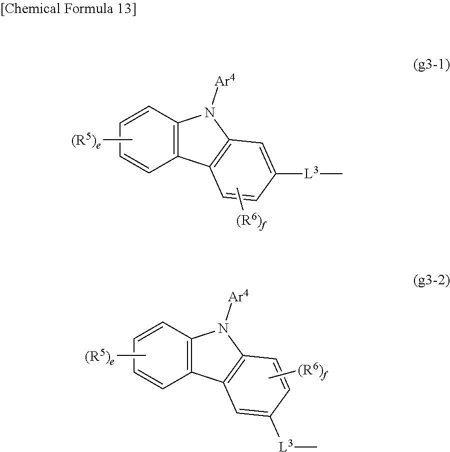
wherein each of R5 and R6 is independently any of a hydrocarbon group having 1 to 6 carbon atoms and an aromatic hydrocarbon group having 6 to 13 carbon atoms; Ar4 is a substituted or unsubstituted phenyl group; e represents an integer of 0 to 4; f represents an integer of 0 to 3; and L3 represents a divalent aromatic hydrocarbon group having 6 to 18 carbon atoms.

72. The light-emitting device according to claim 71, wherein the Ar2 is a group represented by General Formula (g3-1) above.

73. The light-emitting device according to claim 71, wherein the Ar³ is a group represented by General Formula (Ar³-1) or (Ar³-2) below: 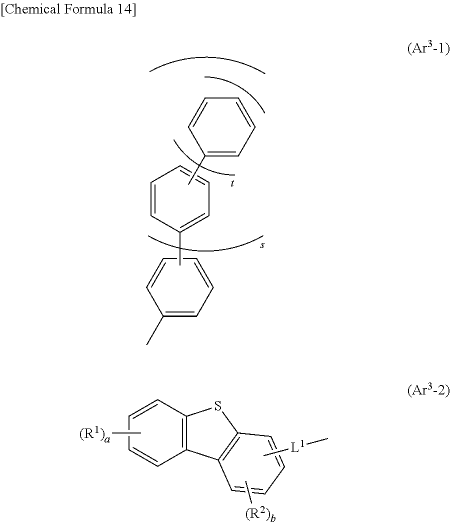
wherein each of s and t is independently 0 or 1; each of R¹ and R² is independently any of a hydrocarbon group having 1 to 6 carbon atoms and an aromatic hydrocarbon group having 6 to 13 carbon atoms; a represents an integer of 0 to 4, b represents an integer of 0 to 3; and L¹ represents a divalent aromatic hydrocarbon group having 6 to 12 carbon atoms.

74. The light-emitting device according to claim 73,
wherein the Ar³ is a group represented by General Formula (Ar³-2) above.

75. The light-emitting device according to claim 61,
wherein the AP is a group represented by Structural Formula (Ar³-1-1) or (AP-1-2) below.

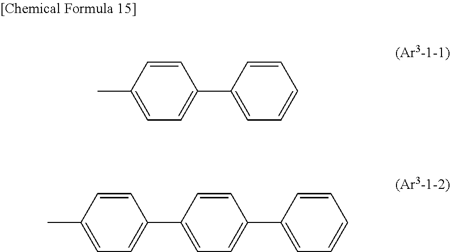

76. The light-emitting device according to claim 75, wherein the Ar³ is a group represented by Structural Formula (Ar³-1-1) above.

77. The light-emitting device according to claim 61,
wherein the organic compound represented by General Formula (G1) is an organic compound represented by General Formula (G2) below: 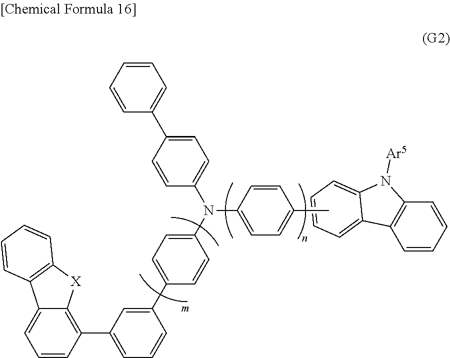
wherein X is an oxygen atom or a sulfur atom; Ar⁵ is a substituted or unsubstituted phenyl group; m is 0 or 1; and n represents an integer of 0 to 2.

78. The light-emitting device according to claim 61,
wherein the second layer is positioned between the first layer and the light-emitting layer.

79. A light-emitting device material used for the second layer of the light-emitting device according to claim 77,
wherein the light-emitting device material is represented by General Formula (G1) above or General Formula (G2) above.

80. An organic compound represented by General Formula (G2) below: 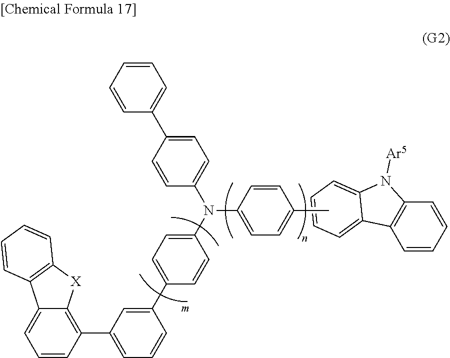
wherein X is an oxygen atom or a sulfur atom; Ar⁵ is a substituted or unsubstituted phenyl group; m is 0 or 1; and n represents an integer of 0 to 2.

81. The organic compound according to claim 80, wherein the X is a sulfur atom.

82. The organic compound according to claim 80, wherein n is 1.
